(19) United States
(12) Patent Application Publication
(10) Pub. No.: US 2021/0283185 A1
(43) Pub. Date: Sep. 16, 2021

(54) METHODS AND MATERIALS FOR IMPROVING TRANSPLANT OUTCOMES

(71) Applicants: Mayo Foundation for Medical Education and Research, Rochester, MN (US); The Brigham and Women's Hospital, Inc., Boston, MA (US)

(72) Inventors: James L. Kirkland, Rochester, MN (US); Tamar Tchkonia, Rochester, MN (US); Stefan Tullius, Boston, MA (US); Ming Xu, Avon, CT (US); Joao Passos, Rochester, MN (US)

(21) Appl. No.: 17/257,923

(22) PCT Filed: Jul. 3, 2019
(86) PCT No.: PCT/US2019/040612

§ 371 Date: Jan. 5, 2021

Related U.S. Application Data
(60) Provisional application No. 62/694,849, filed on Jul. 6, 2018.

(51) Int. Cl. A61K 35/28; A61K 31/506; A61K 31/353; A01K 67/027; A61P 39/00

(52) U.S. Cl. CPC A61K 35/28; A61K 31/506; A61K 31/353; A01K 2267/03; A61P 39/00; A01K 2207/12; A01K 2227/105; A01K 67/0271

(57) ABSTRACT

This document provides methods and materials for treating aging and/or improving transplant outcomes. For example, methods and materials for using one or more senotherapeutic agents to reduce risk of transplant rejection are provided. Non-human animal models for transplant rejection as well as methods for using such non-human animal models to identify agents having the ability to reduce transplant rejection are provided as are non-human animal models for aging and methods for using such non-human animal models to identify agents having the ability to treat aging or the ability to slow the effects of aging.

### CROSS-REFERENCE TO RELATED APPLICATIONS

This application claims the benefit of U.S. Patent Application Ser. No. 62/694,849, filed on Jul. 6, 2018. The disclosure of the prior application is considered part of (and is incorporated by reference in) the disclosure of this application.

### STATEMENT REGARDING FEDERAL FUNDING

This invention was made with government support under NIH grant AG13925 awarded by the National Institutes of Health. The government has certain rights in the invention.

### BACKGROUND

### 1. Technical Field

This document relates to methods and materials for treating aging and/or improving transplant outcomes. For example, this document provides methods and materials for using one or more senotherapeutic agents, one or more toll-like receptor 9 (TLR9) antagonists, or both one or more senotherapeutic agents and one or more TLR9 antagonists to reduce risk of transplant rejection. This document also provides animal models for transplant rejection as well as methods for using such animal models to identify agents having the ability to reduce transplant rejection. In addition, this document provides animal models for aging as well as methods for using such animal models to identify agents having the ability to treat aging or the ability to slow the effects of aging.

### 2. Background Information

There is an insufficient supply of organs for transplantation resulting in long waiting times with increasing morbidity and mortality. Thus, there is strong motivation to meet the increasing demand through optimizing utilization of cells and organs from older donors. Older organs, however, have had high discard rates, and, when they are used, their outcomes are inferior. Moreover, older organs show higher rates of acute rejections. Older recipients, in turn, are more prone to die from immunosuppressive complications.

### SUMMARY

This document provides methods and materials related to treating aging as well as methods and materials related to improving transplant outcomes. For example, this document provides methods and materials for using one or more senotherapeutic agents (e.g., a senolytic agent such as dasatinib), one or more TLR9 antagonists, or both one or more senotherapeutic agents and one or more TLR9 antagonists to reduce risk of transplant rejection (e.g., graft-versus-host disease). In some cases, a donor mammal, a graft obtained from a donor mammal, and/or a recipient mammal can be treated with a composition including one or more senotherapeutic agents, one or more TLR9 antagonists, or both one or more senotherapeutic agents and one or more TLR9 antagonists to reduce risk of transplant rejection. In some cases, one or more senotherapeutic agents, one or more TLR9 antagonists, or both one or more senotherapeutic agents and one or more TLR9 antagonists can be used to maintain graft function, to maintain graft regenerative capacity, and/or to promote graft rejuvenation. For example, a donor mammal, a graft obtained from a donor mammal, and/or a recipient mammal can be treated with a composition including one or more senotherapeutic agents, one or more TLR9 antagonists, or both one or more senotherapeutic agents and one or more TLR9 antagonists to maintain graft function, to maintain graft regenerative capacity, and/or to promote graft rejuvenation.

As described herein, transplanting small numbers of autologous or non-autologous mouse or human senescent cells into young or middle-aged mice can cause early onset of physical dysfunction and age-related diseases and decreased survival compared to controls involving the transplant of non-senescent cells. In addition, treating recipient mice with senolytic agents (e.g., dasatinib and quercetin) and/or TLR9 antagonists at the time of transplantation or after senescent cell transplant-induced symptoms have developed can prevent or alleviates dysfunction caused by transplanting senescent cells and can restore survival to a level observed in mice transplanted with non-senescent cells or non-transplanted mice. As also described herein, young mice transplanted with hearts from old mice can exhibit significantly increased survival when treated with senolytic agents (e.g., dasatinib and quercetin) and/or TLR9 antagonists. Using senolytic agents and/or TLR9 antagonists to reduce senescent cells in cells or organs transplanted from old mammals (e.g., humans) into younger recipient mammals can significantly improve outcomes with reduced morbidity and mortality. In some cases, treating transplant donors, a graft (e.g., cells, tissues, organs, and/or a population of cells not in the form of tissue or an organ to be transplanted), and/or transplant recipients as described herein can expand the supply of cells and organs available for transplantation (e.g., by reducing the need to allocate age-matched cells or organs).

In general, one aspect of this document features methods for providing a recipient mammal with a graft. The methods can include, or consist essentially of, (a1) administering a composition comprising a senotherapeutic agent (e.g., a senolytic agent) to a donor mammal providing the graft before the graft is obtained from the donor mammal, (a2) administering the composition to the recipient mammal, or (a3) contacting the graft with the composition, and (b) providing the recipient mammal with the graft. The recipient mammal can be a human. The graft can be a graft from a human donor. The human donor can be over 55 years of age. The graft can be a tissue graft (e.g., bone marrow). The graft can be an organ graft. The graft can be a population of cells not in the form of tissue or an organ (e.g., hematopoietic stem cells or blood). The senotherapeutic agent can be a senolytic agent. The senolytic agent can be dasatinib or quercetin. The composition can include dasatinib and quercetin. In some cases, the method can include administering the composition including the senotherapeutic agent (e.g., the senolytic agent) to the donor mammal providing the graft before the graft is obtained from the donor mammal. The graft can have improved function or survival within the recipient mammal as compared to a comparable graft obtained from a comparable control donor not administered the composition. In some cases, the method can include administering the composition to the recipient mammal. The composition can be administered to the recipient mammal before the graft is provided to the recipient. The composition can be administered to the recipient mammal after the graft is provided to the recipient. The composition can be administered to the recipient mammal at the same time that the graft is provided to the recipient. The graft can have improved function or survival within the recipient mammal as compared to a comparable graft provided to a comparable recipient mammal not administered the composition. In some cases, the method can include contacting the graft with the composition. The graft can have improved function or survival within the recipient mammal as compared to a comparable graft not contacted with the composition. In some cases, the method can include administering the composition including the senotherapeutic agent (e.g., the senolytic agent) to the donor mammal providing the graft before the graft is obtained from the donor mammal, administering the composition to the recipient mammal, and contacting the graft with the composition. The method also can include (c1) administering a composition comprising a TLR9 antagonist to a donor mammal providing the graft before the graft is obtained from the donor mammal, (c2) administering the composition comprising the TLR9 antagonist to the recipient mammal, or (c3) contacting the graft with the composition comprising the TLR9 antagonist. The TLR9 antagonist can be ODN 2088, SD-101, IMO-2125, CPG10101, or chloroquine.

In another aspect, this document features a non-human mammalian model for aging or transplantation, where the model is a non-human mammal transplanted with a population of senescent cells. The population can include less than 5 million cells. The population of senescent cells can be transplanted into a young wild type non-human mammal, an old wild type non-human mammal, a young wild type high fat fed non-human mammal, or an immunodeficient non-human mammal. The non-human mammal can be a mouse. The model can have a reduced survival time as compared to a comparable non-human mammal not transplanted with the population of senescent cells. The model can include an exogenous graft. The exogenous graft can exhibit an inferior performance within the model as compared to a comparable graft within a non-human mammal not transplanted with the population of senescent cells.

In another aspect, this document features methods for identifying an agent having the ability to reduce the effect of aging within a mammal. The methods can include, or consist essentially of, (a) administering a test agent to a non-human mammalian model for aging or transplantation as described herein, and (b) determining whether or not the test agent reduces an effect of aging within the model. The test agent can reduce an effect of aging within the model, thereby identifying the test agent as being the agent.

In another aspect, this document features methods for identifying an agent having the ability to improve the performance of a transplanted graft within a mammal. The methods can include, or consist essentially of, (a) administering a test agent to a non-human mammalian model for aging or transplantation as described herein, and (b) determining whether or not the test agent improves the performance of the exogenous graft within the model. The test agent can improve the performance of the exogenous graft within the model, thereby identifying the test agent as being the agent.

In another aspect, this document features methods for detecting senescent cells where the methods can include, or consist essentially of, determining the presence or absence of cell-free mitochondrial DNA (cf-mt-DNA) in a sample, where the presence of cf-mt-DNA in the sample indicates that the sample contains senescent cells, and where the absence of cf-mt-DNA in the sample indicates that the sample lacks senescent cells. The determining step can include a polymerase chain reaction (PCR) technique. The PCR technique can be real time PCR. The sample can be obtained from a recipient mammal with a graft. The sample can be obtained from a donor mammal providing a graft to be transplanted into a recipient mammal. The sample can be obtained from a graft to be transplanted into a recipient mammal. The mammal can be a human. The graft can be a tissue graft (e.g., bone marrow). The graft can be an organ graft. The graft can be a population of cells not in the form of tissue or an organ (e.g., hematopoietic stem cells or blood). The method can include determining the presence of the cf-mt-DNA in the sample. The method can include determining the absence of the cf-mt-DNA in the sample.

In another aspect, this document features methods for detecting senescent cells where the methods can include, or consist essentially of, determining the presence or absence of an elevated level of cf-mt-DNA in a sample, where the presence of the elevated level of cf-mt-DNA in the sample indicates that the sample contains senescent cells, and where the absence of the elevated level of cf-mt-DNA in the sample indicates that the sample lacks senescent cells. The determining step can include a PCR technique. The PCR technique can be real time PCR. The sample can be obtained from a recipient mammal with a graft. The sample can be obtained from a donor mammal providing a graft to be transplanted into a recipient mammal. The sample can be obtained from a graft to be transplanted into a recipient mammal. The mammal can be a human. The graft can be a tissue graft (e.g., bone marrow). The graft can be an organ graft. The graft can be a population of cells not in the form of tissue or an organ (e.g., hematopoietic stem cells or blood). The method can include determining the presence of the elevated level of cf-mt-DNA in the sample. The method can include determining the absence of the elevated level of cf-mt-DNA in the sample. The sample can be a liquid. The elevated level can be greater than 20,000 copies per mL of sample.

In another aspect, this document features methods for providing a recipient mammal with a graft. The methods can include, or consist essentially of, (a1) administering a composition comprising a TLR9 antagonist to a donor mammal providing the graft before the graft is obtained from the donor mammal, (a2) administering the composition to the recipient mammal, or (a3) contacting the graft with the composition, and (b) providing the recipient mammal with the graft. The recipient mammal can be a human. The graft can be a graft from a human donor. The human donor can be over 55 years of age. The graft can be a tissue graft (e.g., bone marrow). The graft can be an organ graft. The graft can be a population of cells not in the form of tissue or an organ (e.g., hematopoietic stem cells or blood). The TLR9 antagonist can be ODN 2088, SD-101, IMO-2125, CPG10101, or chloroquine. For example, the composition can include ODN 2088. The method can include administering the composition to the donor mammal providing the graft before the graft is obtained from the donor mammal. The graft can have improved function or survival within the recipient mammal as compared to a comparable graft obtained from a comparable control donor not administered the composition. The method can include administering the composition to the recipient mammal. The composition can be administered to the recipient mammal before the graft is provided to the recipient. The composition can be administered to the recipient mammal after the graft is provided to the recipient. The composition can be administered to the recipient mammal at the same time that the graft is provided to the recipient. The graft can have improved function or survival within the recipient mammal as compared to a comparable graft provided to a comparable recipient mammal not administered the composition. The method can include contacting the graft with the composition. The graft can have improved function or survival within the recipient mammal as compared to a comparable graft not contacted with the composition. The method can include administering the composition to the donor mammal providing the graft before the graft is obtained from the donor mammal, administering the composition to the recipient mammal, and contacting the graft with the composition. The method also can include (c1) administering a composition comprising a senotherapeutic agent to a donor mammal providing the graft before the graft is obtained from the donor mammal, (c2) administering the composition comprising the senolytic agent to the recipient mammal, or (c3) contacting the graft with the composition comprising senolytic agent. The senotherapeutic agent can be a senolytic agent. The senolytic agent can be dasatinib or quercetin.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1. Transplanting small numbers of senescent cells induces physical dysfunction in younger mice. (a) Experimental design for transplantation and physical function measurements. (b,c) Representative images of LUC activity of various organs from LUC-negative male mice (n=3) 5 days post-transplantation with SEN (induced by radiation) and CON preadipocytes from LUC-positive transgenic mice. Scale bars, 10 mm. (d j) Maximal walking speed (relative to baseline) (d), hanging endurance (e), grip strength (f), daily activity (g), treadmill endurance (h), food intake (i), and change in body weight (BW) (j) of 6-month-old male C57BL/6 mice 1 month after being injected with PBS, 1×10⁶ non-senescent control (1M CON), 0.2×10⁶ SEN (0.2M SEN), 0.5×10⁶ SEN (0.5M SEN), or 1×10⁶ SEN (1M SEN) preadipocytes (n=6 for all groups). Results are means±s.e.m. (k-m). SA-βgal⁺ cell numbers (n=6) (k), p16^{Ink4a} mRNA levels (n=7) (l), and cells from recipient mice that were TAF⁺ (>2 TAFs/nucleus) and LUC″ (n=4 mice) (m) in 6-month-old male wildtype (LUC) C57BL/6 mice 2 months after being transplanted with 1×10⁶ SEN or CON transgenic constitutively-expressing LUC (LUC⁺) preadipocytes from transgenic mouse donors. Results are shown as box and whiskers plots, where a box extends from the 25th to 75th percentile with the median shown as a line in the middle, and whiskers indicate smallest and largest values. *P<0.05; ANOVA with Tukey's post-hoc comparison (d-j) and two-tailed, unpaired Student's t-test (k-m).

FIG. 2. Senescent preadipocytes develop a SASP. (a,b) Representative images of SEN (induced by radiation) and CON primary mouse preadipocytes of cellular SA-βgal activity; Scale bar, 400 μm and γH2AX staining; Scale bar, 100 μm with DAPI staining (blue). (c) Secreted cytokine protein levels in CM from SEN and CON mouse preadipocytes (n=5 for both groups). (d) The relative mRNA abundance of key SASP components of SEN (induced by radiation) and CON primary mouse preadipocytes (n=5 for both groups). (e) The relative mRNA abundance of key SASP components of naturally-accumulating p16^{Ink4a} positive (GFP⁺) and negative (GFP⁻) cells isolated from adipose tissue from 24-month-old INK-ATTAC mice by flow cytometry (n=3 for both groups). (f) Secreted cytokine protein levels in CM from CON, IRA (senescence induced by radiation), or DOXO (senescence induced by doxorubicin) primary human preadipocytes (n=3 for all groups). All results are shown as mean±s.e.m. (d-f) P<0.05 between groups for all genes; Two-tailed Student's t-tests.

FIG. 3. Transplanted senescent cells are mainly localized in intraperitoneal adipose tissue. (a) Representative BLI images of SEN cell-transplanted female mice. Scale bar, 10 mm. (b) Luminescence of different organs 5 days after transplantation (n=3). (c) Luminescence of mice at different times after transplantation (n=5). (d) LUC activity for the same number of SEN (induced by radiation) and CON preadipocytes isolated from LUC transgenic mice. Results are shown as mean±s.e.m.

FIG. 4. Senescent cells induce physical dysfunction in young mice. (a) Maximal walking speed of 6-month-old male C57BL/6 mice before and one month after being transplanted with PBS, 1×10⁶ CON (CON), 0.2×10⁶ SEN (0.2M SEN), 0.5×10⁶ SEN (0.5M SEN), or 1×10⁶ SEN (1M SEN) preadipocytes (n=6 for all groups). (b) Maximal walking speed (relative to baseline) of 6-month-old male C57BL/6 mice 1 month after being transplanted with 0.5×10⁶ SEN (0.5M SEN) or CON preadipocytes (n=21). (c) Maximal walking speed (relative to baseline) of 15-month-old male C57BL/6 mice 2 weeks after being transplanted with 1×10⁶ SEN, CON preadipocytes, or PBS (n=6 for SEN and CON; n=7 for PBS). (d) Maximal walking speed (relative to baseline) at different time points of 5-month-old male C57BL/6 mice after being transplanted with 1×10⁶ SEN or CON preadipocytes (n=10 for SEN; n=14 for CON). (e) Relative mRNA levels of F4/80 (n=7) and (f) representative images of F4/80 immunostaining of visceral adipose tissue from mice 60 days after transplantation. Scale bar, 400 μm. All results are shown as box and whiskers plots, where a box extends from the 25th to 75th percentile with the median shown as a line in the middle, and whiskers indicate smallest and largest values. *:P<0.05; Two-tailed unpaired Student's t-tests.

FIG. 5. Senescent cells induce muscle dysfunction in young mice. (a) Relative mRNA levels for target genes (n=7) and (b) representative images of F4/80 immunostaining in quadriceps muscle from 5-month-old male C57BL/6 mice 2 mo after being transplanted with 1×10⁶ SEN or CON preadipocytes. Scale bar, 200 μm. All results are shown as box and whiskers plots, where a box extends from the 25th to 75th percentile with the median shown as a line in the middle, and whiskers indicate smallest and largest values. *:P<0.05; Two-tailed unpaired Student's t-tests.

FIG. 6. IL10^{-/-} mice have increased cellular senescence. (a) Relative mRNA levels for target genes (n=7), (b) SA-βgal⁺% (n=8), and (c) cells % that were TAF⁺ (>2 TAFs/nucleus) (n=4) in visceral adipose tissue collected from 8-9-month-old IL10^{-/-} and wildtype C57BL/6 mice (both sexes). Results are shown as box and whiskers plots, where a box extends from the 25th to 75th percentile with the median shown as a line in the middle, and whiskers indicate smallest and largest values. *:P<0.05; Two-tailed unpaired Student's t-tests.

FIG. 7. Aging exacerbates effects of senescent cell transplantation. (a) Experimental design for transplantation and physical function measurements. (b-h) Maximal walking speed (relative to baseline) (b), hanging endurance (c), grip strength (d), body weight change from baseline (e), treadmill endurance (f), daily activity (g), and food intake (h) of 17-month-old male C57BL/6 mice 1 month after being injected with 0.5×10⁶ SEN or CON preadipocytes (n=8 for both groups). (i) Percent changes in RotaRod (in 6-month-old mice, n=21 for both SEN and CON; in 17-month-old mice, n=22 for SEN, n=20 for CON) and hanging test (in 6-month-old mice, n=6 for both SEN and CON; in 17-month-old mice, n=8 for both SEN and CON) in mice transplanted with 0.5×10⁶ SEN cells relative to the average of mice transplanted with 0.5×10⁶ CON cells at both ages. Results are shown as box and whiskers plots, where a box extends from the 25th to 75th percentile with the median shown as a line in the middle, and whiskers indicate smallest and largest values. (j) One year survival curves of 17-month-old non-transplanted mice (n=33, N/A) and mice transplanted with 0.5×10⁶ SEN (n=23) or CON (n=24) preadipocytes. (k) Tumor burden, disease burden, and inflammation at death are shown as means±s.e.m. after transplanting SEN or CON cells (n=10 for SEN, n=7 for CON). (l) Causes of death (n=10 for SEN, n=7 for CON). *P<0.05; Two-tailed unpaired Student's t-test (b-i), Cox proportional hazard regression model (j) and chi-square and Fisher's exact tests (1).

FIG. 8. Senescent cells induce physical dysfunction in older mice. (a-f) Maximal walking speed (relative to baseline), hanging endurance, grip strength, body weight (change from baseline), daily activity, and food intake of 17-month-old male C57BL/6 mice 1 month after being transplanted with 0.5×10⁶ SEN or CON preadipocytes. (a-d) n=14 for SEN, n=12 for CON; (e-f) n=8 for both groups. (g-l) Maximal walking speed (relative to baseline), hanging endurance, grip strength, body weight (change from baseline), daily activity, and food intake of 17-month-old female C57BL/6 mice 1 month after being transplanted with 0.5×10⁶ SEN or CON preadipocytes. (g j) n=9 for both groups; (k-l) n=8 for both groups. All results are shown as box and whiskers plots, where a box extends from the 25th to 75th percentile with the median shown as a line in the middle, and whiskers indicate smallest and largest values. *:P<0.05; Two-tailed unpaired Student's t-tests.

FIG. 9. Senescent cells reduce resilience to metabolic stress in mice. (a) Experimental design for transplantation and physical function measurements. (b-h) Maximal walking speed (relative to baseline) (b), hanging endurance (c), grip strength (d), daily activity (e), food intake (f), body weight change from baseline (g), and treadmill endurance (h) of 8-month-old male C57BL/6 mice 1 month after being on HFD and injected with 0.4×10⁶ SEN or CON preadipocytes (n=6 for both groups). (i) Percent changes in RotaRod (on NCD, n=21 for both SEN and CON; on HFD, n=12 for both SEN and CON) and hanging test (on NCD, n=6 for both SEN and CON; on HFD, n=6 for both SEN and CON) in mice transplanted with 0.4-0.5×10⁶ SEN cells relative to the average of mice transplanted with 0.4-0.5×10⁶ CON cells. (j) Experimental design for transplantation and physical function measurements. (k-q) Maximal walking speed (relative to baseline) (k), hanging endurance (l), grip strength (m), body weight change from baseline (n), treadmill endurance (o), daily activity (p), and food intake (q) of 8-month-old male C57BL/6 650 mice 1 month after being on HFD and injected with 1×10⁶ SEN or CON autologous ear fibroblasts (n=10 for both groups). All results are shown as box and whiskers plots, where a box extends from the 25th to 75th percentile with the median shown as a line in the middle, and whiskers indicate smallest and largest values. *P<0.05; Two-tailed unpaired Student's t-tests (a-q).

FIG. 10. Senescent cells induce physical dysfunction in SCID-beige mice. (a) Experimental design for transplantation and physical function measurements. (b-d) Maximal walking speed (relative to baseline), hanging endurance, and grip strength of 2-month-old male SCID-beige mice 2 weeks after being transplanted with PBS, 0.5×10⁶ SEN, or 0.5×10⁶ CON human senescent preadipocytes (n=8 for CON and SEN, n=4 for PBS). All results are shown as box and whiskers plots, where a box extends from the 25th to 75th percentile with the median shown as a line in the middle, and whiskers indicate smallest and largest values. *:P<0.05; Two-tailed unpaired Student's t-tests.

FIG. 11. D+Q reduces senescent cells in obese human adipose tissue. (a) Percent p16^{INK4A-high} cells of human omental adipose tissue explants obtained from age-matched obese (n=6; 2 males and 4 females; age=44.9±8.6 years; BMI=46.7±10.1 kg/m²) or non-obese subjects (n=4; 1 male and 3 females; age=47±15.4 years; BMI=26.4±2.8 kg/m²). (b) Representative images of TUNEL⁺ cells (%; arrows indicate TUNEL⁺ cells) and total cells (n=4) in adipose tissue explants from obese subjects treated with D+Q (1 μM+20 μM, respectively) or V for 48 hours. Scale bar, 100 μm. (c) The percent of p16^{INK4A-high} cells and total cell number per field (n=3) in adipose tissue explants before and 48 hours after being treated with V. (d) Correlation curves for cellular senescence markers (% p16^{Ink4a-high} cells, % TAF⁺ cells, and % SA-βgal⁺ cells) in human adipose tissue. (a-b) Results are shown as box and whiskers plots, where a box extends from the 25th to 75th percentile with the median shown as a line in the middle, and whiskers indicate smallest and largest values. (c) Results are shown as means±s.e.m. *:P<0.05; Two-tailed unpaired Student's t-tests (a-c) and Pearson's correlation coefficients (d).

FIG. 12. Dasatinib plus quercetin (D+Q) reduces senescent cell abundance and decreases pro-inflammatory cytokine secretion in human adipose tissue. (a) Experimental design. (b) Percent TAF⁺ cells (n=5). Blue arrows indicate TAFs. Scale bars, 5 μm. (c) Percent p16^{Ink4A-high} cells (red arrows), percent p16^{INK4A+} cells (expressing any detectable level of p16^{INK4A}, green arrows), percent p16^{INK4A-} cells (black arrows), and cell number per field (n=6). Scale bar, 100 μm. (d) Percent SA-βgal⁺ cells (red arrows) (n=6). Scale bar, 100 μm. (e) Percent cleaved caspase-3⁺ cells (red arrows) (n=5). Scale bar, 100 μm. (f) Secreted cytokine and adipokine levels in conditioned media (CM) (n=8). Results are means±s.e.m. (g) The relative mRNA abundance of key senescence associated secretory phenotype (SASP) components and markers for adipose tissue function (n=7). All results are shown as box and whiskers plots, where a box extends from the 25th to 75th percentile with the median shown as a line in the middle, and whiskers indicate smallest and largest values. *P<0.05; Two-tailed Student's t-tests (a-g).

FIG. 13. D+Q has little acute effect on macrophages in obese human adipose tissue. (a) Representative images for p16^{INK4a} co-stained with F4/80 (scale bar, 10 μm), p16⁺; F4/80⁻ cells %, p16⁻; F4/80⁺ cells %, and p16⁺; F4/80⁺ cells % in adipose tissue explants treated with D+Q or V for 48 (n=4). (b) Representative images of CD68 immunostaining (Scale bar, 200 μm) and the relative mRNA levels for EMR-1 (n=7) in adipose tissue explants. Results are shown as box and whiskers plots, where a box extends from the 25th to 75th percentile with the median shown as a line in the middle, and whiskers indicate smallest and largest values. *:P<0.05; Two-tailed unpaired Student's t-tests.

FIG. 14. Senescent preadipocytes induce pro-inflammatory cytokine secretion by human adipose tissue. (a) Experimental design. (b) Secreted cytokine and adipokine levels in CM from human subcutaneous adipose tissue explants previously treated with CM collected from SEN (induced by radiation) or CON human preadipocytes and blank culture flasks containing no cells (Blank CM) (n=4 for SEN; n=4 for CON; n=2 for Blank CM). All results are mean±s.e.m; Two-tailed Student's t-tests.

FIG. 15. D+Q reduces pro-inflammatory cytokine secretion by human adipose tissue from obese subjects. (a) Secreted cytokine and adipokine levels in CM from adipose tissue explants treated with D+Q (1 μM+20 μM) or V for 48 hours (n=8). Results are shown as box and whiskers plots, where a box extends from the 25th to 75th percentile with the median shown as a line in the middle, and whiskers indicate smallest and largest values. (b) Secreted cytokine levels in CM (percentage relative to V) from adipose tissue explants treated with V, D alone (1 μM), Q alone (20 μM), or D+Q (1 μM+20 μM) for 48 hours (n=3). Results are shown as means±s.e.m. *:P<0.05; Two-tailed unpaired Student's t-tests.

FIG. 16. Eliminating senescent cells both prevents and alleviates physical dysfunction. (a) Experimental design for transplantation and physical function measurements. (b) Representative images of LUC activity in mice 2 days after the last treatment. Scale bars, 15 mm. (c) Luminescence of transplanted cells as percent relative to the average of mice treated with V (n=16 for SEN-DQ vs. SEN-V; n=13 for CON-DQ vs. CON-V). (d-f) Maximal walking speed (relative to baseline) (d), hanging endurance (e), and grip strength (f) of 5-month-old male C57BL/6 mice 1 month after the last drug treatment (n=7 for SEN-V, CON-V, and SEN-DQ; n=6 for CON-DQ). (g) Experimental design for transplantation and physical function measurements. (h j) Maximal walking speed (relative to baseline) (h), hanging endurance (i), and grip strength (j) of 5-month-old male C57BL/6 mice 2 weeks after the last drug treatment (n=10 for SEN-DQ and SEN-V; n=14 for CON-V). All results are shown as box and whiskers plots, where a box extends from the 25th to 75th percentile with the median shown as a line in the middle, and whiskers indicate smallest and largest values. *P<0.05; Two-tailed Student's t-tests (a-j).

FIG. 17. Clearing senescent cells alleviates physical dysfunction. (a) Maximal walking speed (relative to baseline) of 5-month-old male C57BL/6 mice at different time points after being transplanted with 1×10⁶ SEN preadipocytes and then treated with D+Q or V (n=10 for both groups). (b-f). Naturally-aged mice were treated with AP20187 (AP) every 2 weeks. (b) Schematic of the INK-ATTAC construct. (c) Maximal walking speed (relative to baseline), (d) hanging endurance, and (e) body weight change of 23-25-month-old INK-ATTAC^{+/-} female C57BL/6 mice or age-matched wildtype (WT) littermates 10 weeks after the first treatment with AP20187 (n=18 for INK-ATTAC; n=14 for WT). All results are shown as box and whiskers plots, where a box extends from the 25th to 75th percentile with the median shown as a line in the middle, and whiskers indicate smallest and largest values. (f) Survival curves of INK-ATTAC^{+/-} (n=29) and WT mice (n=32) treated with AP20187 bi-weekly. *:P<0.05; Two-tailed unpaired Student's t-tests (a-e) and Cox proportional hazard regression model (f).

FIG. 18. Senolytics extend both health- and life-span in aged mice. (a) Experimental design for physical function measurements in 20-month-old male mice treated with D+Q once every 2 weeks (bi-weekly) for 4 months. (b-h) Maximal walking speed (relative to baseline) (b), hanging endurance (c), grip strength (d), body weight change from baseline (e), treadmill endurance (f), daily activity (g), and food intake (h) of 20-month-old male C57BL/6 mice 4 month after drug initiation (n=20 for D+Q; n=13 for V). (i) The relative mRNA abundance for target genes of visceral adipose tissue from 6-month-old non-treated (6m, n=7), 24-month-old V-treated (24m-V, n=8), and 24-month-old D+Q-treated (24m-DQ, n=8) mice. (j) Experimental design for lifespan analyses. (k,l) Post-treatment survival curves (k) and whole-life survival curves (l) of C57BL/6 mice treated bi-weekly with D+Q (n=71; 40 males, 31 females) or V (n=76; 41 males, 35 females) starting at 24-27 months of age. Median survival is indicated for all curves. (m) Maximal walking speed and hanging endurance averaged over the last 2 months of life and lifespan for the longest living mice (top 40%) in both groups for both sexes. For male mice, n=12 for D+Q and n=12 for V. For female mice, n=13 for D+Q and n=13 for V. (n) Disease burden and tumor burden at death. For both sexes, n=59 for D+Q, n=62 for V. For males, n=30 for D+Q, n=29 for V. For females, n=29 for D+Q, n=33 for V. (b-i, m) Results are shown as box and whiskers plots, where a box extends from the 25th to 75th percentile with the median shown as a line in the middle, and whiskers indicate smallest and largest values. (n) Results are shown as mean±s.e.m. *P<0.05; n.s., not significant; Two-tailed Student's t-tests (b-i, m-n) and Cox proportional hazard regression model (k-l).

FIG. 19. Senolytic drugs did not affect circulating testosterone in old mice. (a) Circulating testosterone levels in naturally-aged 24-month-old male C57BL/6 mice treated with D+Q (n=9) or V bi-weekly (n=10) for 1 month. (b) Circulating testosterone levels in naturally-aged 20-month-old male C57BL/6 mice treated with D+Q or V bi-weekly for 4 months (n=10 for both groups). All results are means±s.e.m.

FIG. 20. Senolytic drugs increase remaining lifespan without extending morbidity. C57BL/6 mice were treated with D+Q or V every 2 weeks starting at 24-27 months of age. (a) Survival curves of C57BL/6 male mice treated with D+Q (n=40) or V (n=41) bi-weekly. Life-long and post-treatment (post-tx) median survival is indicated. (b) Survival curves of C57BL/6 female mice treated with D+Q (n=31) or V (n=35) bi-weekly. Life-long and post-treatment (post-tx) median survival is indicated. (c) Maximal walking speed of each mouse before initiating intermittent D+Q treatment (baseline, 0) and monthly thereafter until death in both male and female mice. Averages for both groups at each time point are indicated. For male mice, n=29 for D+Q and n=31 for V. For female mice, n=31 for D+Q and n=33 for V. (d) Maximal walking speed and hanging endurance averaged over the last 2 months of life in all mice in the D+Q-treated and V-treated groups of both sexes. For male mice, n=29 for D+Q and n=30 for V. For female mice, n=31 for D+Q and n=30 for V. All results are shown as box and whiskers plots, where a box extends from the 25th to 75th percentile with the median shown as a line in the middle, and whiskers indicate smallest and largest values. *:P<0.05; Cox proportional hazard regression model (a,b); two-tailed unpaired Student's t-tests (d).

FIG. 21. Senolytic drugs did not substantially alter the ultimate cause of death in old mice. Causes of death of C57BL/6 mice treated with D+Q or V bi-weekly starting at 24-27 months of age. No significant difference was found using either chi-square or Fisher's exact tests.

FIG. 22. Treatment with senolytic agents prolongs cardiac allograft survival. Hearts from old C57BL/6 mice were transplanted into young DBA/2J mice w/o immunosuppression. Senolytics (D+Q) were administered or animals remained untreated (n=4/group; *P=0.0280; data represent two independent sets of experiments).

FIG. 23. Mechanisms of transferring rejuvenation and aging. Several mediators of rejuvenation and aging may be transferred between young and old individuals. Cells may not only be capable of secreting factors that affect surrounding cells, but may also integrate into and contribute to tissue and organ function. Extracellular vesicles containing nucleic acid, protein, and lipid products are capable of fusing with target cells to influence cellular behavior. Soluble factors secreted from cells modulate signaling pathways implicated in the regulation of aging.

FIG. 24. Potential opportunities for therapeutic intervention. (A) Older transplant donors may be treated with senolytic agents prior to organ donation. Senolytics with a minimal side effect profile are desirable for such applications. (B) Treatment of the allograft following organ procurement and prior to transplantation provide additional opportunities for the administration of senolytics, potentially via the addition of such compounds to organ preservation solutions. The recent development of novel organ preservation methods allowing for extended duration of ex vivo organ maintenance may permit not only senolytic treatment, but also additional means of therapeutic intervention. (C) Transplant recipients can also be treated with senolytics or senomorphics at the time of transplantation or subsequent to transplantation. Initial studies of these compounds indicate that the extent of their direct effects is specific for senescent cells.

FIG. 25. Old dendritic cells exhibit an activated phenotype and promote Th1 and Th17 T cell responses. Single cell suspensions of lymph nodes and spleens from old and young C57BL/6 mice were labeled with anti-CD11c, anti-CD11b, anti-WIC class II, anti-CD40, anti-CD80, and anti-CD86. (A) The frequency of CD11b⁺CD11c⁺DCs in lymph nodes, and (B) the expression of costimulatory molecules by splenic DCs were assessed by flow cytometry. (C) Proliferative capacities of CD4⁺ T co-cultured with varying ratios of old and young DCs and (D) for increasing co-culture times were determined using [³H]TdR incorporation. (E) Young CD4⁺ T cells were co-cultured with CD11b⁺CD11c⁺DCs isolated from young and old mice and (F) pro-inflammatory cytokine expression was assessed by flow cytometry and ELISA; results are representative of at least three separate experiments.

FIG. 26. Old dendritic cells impair cardiac allograft survival in young DBA/2J mice. (A) 2×10⁶ CD11b⁺CD11c⁺DCs were sorted from old and young C57BL/6 mice and administered i.v. into young DBA/2J mice 7 days prior to allogeneic cardiac transplants. (B) Kaplan-Meier analysis of old and young C57BL/6 cardiac allografts in untreated DBA/2J recipients compared with those that had received adoptively transferred old and young CD11b⁺CD11c⁺ B6 DCs. Comparison of survival curves was performed using the log-rank test; there were 7-9 animals/group. (C) By day 11 after transplantation, grafts were procured, and perfused and 5-mm sections were stained with H&E for pathological evaluation. Mean scores±SD combining 3 independent experiments are shown (n=10/group).

FIG. 27. Systemic cf-mt-DNA increases drastically upon IRI in old mice and promotes DC maturation through TLR9. (A) Ischemia reperfusion injury was induced by clamping the renal pedicle of young and old C57Bl/6 (2 and 18 months) mice for 22 minutes, respectively. IRI and naïve animals were sacrificed after 48 hours and kidneys were procured. The Picture shows macroscopic appearance of kidneys directly after IRI (B) Cell free mitochondrial DNA (cf-mt-DNA) was quantified in the plasma by real time PCR according to standard curve results. (C) Young and old plasma DNA was added to cultures of young DCs and costimulatory cytokine expression was assessed by flow cytometry with or in absence of a TLR9 antagonist. (D) Different concentrations of cf-mt-DNA isolated from young and old mice was then added to young DC cultures, costimulatory molecule expression was analyzed by flow cytometry with or in absence of a TLR9 antagonist, and it-6 production was measured by ELISA. (E) Immediately after receiving a fully mismatched cardiac allograft from an 18 month old C57Bl/6 donor mouse, 3 months old DBA2/J recipient mice were treated i.p. with a TLR9 antagonist.

FIG. 28. Senescent cells accumulate with aging and are a source of cf-mt-DNA with aging. Skin, hearts, and kidneys were procured from old and young C57BL/6 mice and embedded in paraffin. (A) Skin and hearts were cut into slides and co-stained for p16^{1a4}a, p2l^{Cip1}, and DAPI; (B) frozen slides of kidneys were made and subsequently stained for sa-β-gal. The percentage of senescent cells was defined as the number of (A) p16/p21 double positive cells or (B) sa-β-gal positive cells of DAPI stained cells using a confocal microscope. (C) Mouse adipocytes were isolated from C57BL/6 mice and senescence induced using 30 serial passages of 10 Gy irradiation. Cf-mt-DNA levels were measured in supernatants by real time PCR comparing senescent and naïve cell cultures.

FIG. 29. Old human organ donors displayed increased systemic levels of cf-mt-DNA activating DCs. (A) Cell free mitochondrial DNA (cf-mt-DNA) was quantified in the plasma of young (<35 years) and old (>55 years) organ donors by RT-PCR using TAQMAN primers for mt-Co3 and mt-nd6. (B) Dendritic cells were differentiated from isolated PBMC, stimulated with human mt-DNA (100 μm/mL) and costimulatory molecule expression was analyzed using flow cytometry.

FIG. 30. Senolytics decrease the number of senescent cells, reduce cf-mt-DNA levels, ameliorate systemic inflammatory immune response after IRI and prolong cardiac allograft survival. (A) Old C57/B6 mice were treated with senolytics (D+Q) on 3 successive days/week. After one month, kidney, heart, and skin were harvested, stained, and the percentage of senescent cells assessed as described in FIG. 28. (B) Systemic levels of p16^{Ink4a} and cf-mt-DNA were measured by real time PCR and calculated relative to GAPDH expression (C) Young and old C57BL/6 mice were treated with senolytics for 3 successive days/week for 1 month. Subsequently, IRI was induced in young and old animals; IL-17 and IFN-γ expression of CD4⁺ and CD8⁺ T cells assessed by flow cytometry (D) Old donor C57BL/6 mice of cardiac allografts were treated with a single dose of D+Q prior to transplantation and allograft survival was monitored by daily palpations.

FIG. 31. Senescent cell build-up and cf-mt-DNA accumulation at the crossroad of immunogenicity, inflammation, and alloimmunity.

FIG. 32. Characteristics of deceased organ donors.

### DETAILED DESCRIPTION

Transplant donor and transplant recipient age are factors that can influence transplantation outcomes. Aside from age-associated differences in intrinsic graft function and alloimmune responses, the ability of young and old cells to either exert rejuvenating or aging effects may also apply to the transplantation of cells (e.g., hematopoietic stem cells), organ (e.g., solid organ) transplants, and/or transplantation of blood (e.g., a blood transfusion).

This document provides methods and materials for improving transplant outcomes. In some cases, one or more senotherapeutic agents can be used to reduce risk of transplant rejection (e.g., graft-versus-host disease). For example, a donor mammal, a graft obtained from a donor mammal, and/or a recipient mammal can be treated with a composition including one or more senotherapeutic agents, one or more TLR9 antagonists, or both one or more senotherapeutic agents and one or more TLR9 antagonists to reduce risk of transplant rejection. In some cases, one or more senotherapeutic agents, one or more TLR9 antagonists, or both one or more senotherapeutic agents and one or more TLR9 antagonists can be used to maintain graft function, to maintain graft regenerative capacity, and/or to promote graft rejuvenation. For example, a donor mammal, a graft obtained from a donor mammal, and/or a recipient mammal can be treated with a composition including one or more senotherapeutic agents, one or more TLR9 antagonists, or both one or more senotherapeutic agents and one or more TLR9 antagonists to maintain graft function, to maintain graft regenerative capacity, and/or to promote graft rejuvenation. In some cases, the methods and materials described herein can be used to improve transplant outcomes in sex-mismatched transplantation donor/recipient pairs. In some cases, the methods and materials described herein can be used to improve transplant outcomes in age-mismatched transplantation donor/recipient pairs.

A composition containing one or more senotherapeutic agents, one or more TLR9 antagonists, or both one or more senotherapeutic agents and one or more TLR9 antagonists can include any appropriate senotherapeutic agent(s) and/or any appropriate TLR9 antagonists. In some cases, a senotherapeutic agent can be a senolytic agent (i.e., an agent having the ability to induce cell death in senescent cells). Examples of senolytic agents that can be used as described herein (e.g., to improve transplantation outcomes) can include, without limitation, dasatinib, quercetin, navitoclax, A1331852, A1155463, fisetin, luteolin, geldanamycin, tanespimycin, alvespimycin, piperlongumine, panobinostat, FOX04-related peptides, nutlin3a, flavonoids (e.g., flavonols), and derivatives thereof. In some cases, a senotherapeutic agent can be a senomorphic agent (i.e., an agent having the ability to suppress senescent phenotypes without cell killing). Examples of senomorphic agents that can be used as described herein (e.g., to improve transplantation outcomes) can include, without limitation, ruxolitinib, metformin, and rapamycin. In some cases, a senotherapeutic agent used as described herein can be an orally-active senotherapeutic agent. A senotherapeutic agent can be any appropriate type of molecule. For example, a senotherapeutic agent can be a small molecule. In some cases, one, two, three, four, five or more different senotherapeutic agents can be used in combination or sequentially to improve transplant outcomes in a mammal (e.g., a human).

In some cases, a TLR9 antagonist can recognize (e.g., can bind to) unmethylated CpG oligonucleotide (ODN) sequences. Examples of TLR9 antagonists that can be used as described herein (e.g., to improve transplantation outcomes) include, without limitation, ODN 2088, SD-101, IMO-2125, CPG10101, and chloroquine. A TLR9 antagonist can be any appropriate type of molecule. For example, a TLR9 antagonist can be a small molecule. In some cases, one, two, three, four, five or more different TLR9 antagonists can be used in combination or sequentially to improve transplant outcomes in a mammal (e.g., a human).

When treating a donor mammal and/or a recipient mammal as described herein (e.g., to improve transplant outcomes), the mammal can be any appropriate mammal. In some cases, a mammal can be an older mammal (e.g., a human over 55 years of age). Examples of mammals that can be treated using a composition containing one or more senotherapeutic agents, one or more TLR9 antagonists, or both one or more senotherapeutic agents and one or more TLR9 antagonists as described herein include, without limitation, humans, non-human primates such as monkeys, dogs, cats, horses, cows, pigs, sheep, mice, rats, hamsters, guinea pigs, and goats. In some cases, a composition containing one or more senotherapeutic agents, one or more TLR9 antagonists, or both one or more senotherapeutic agents and one or more TLR9 antagonists can be administered to a human (e.g., a human donor and/or a human recipient) to improve transplant outcomes.

When treating a graft (e.g., cells, tissues, organs, and/or a population of cells not in the form of tissue or an organ) to be transplanted from a mammal as described herein (e.g., to improve transplant outcomes), the mammal can be any appropriate mammal. In some cases, a graft can be from a deceased mammal. In some cases, a graft can be from a mammal on life support. In some cases, a graft can be from an older mammal (e.g., a human over 55 years of age). Examples of mammals from which a graft can be obtained include, without limitation, humans, non-human primates such as monkeys, dogs, cats, horses, cows, pigs, sheep, mice, rats, hamsters, guinea pigs, and goats. In some cases, a composition containing one or more senotherapeutic agents, one or more TLR9 antagonists, or both one or more senotherapeutic agents and one or more TLR9 antagonists can be administered to a graft obtained from a human to improve transplant outcomes.

When treating a donor mammal and/or a recipient mammal as described herein (e.g., to improve transplant outcomes) or when treating the graft itself, any appropriate type of graft can be used. A graft can be an autograft or an allograft. In cases where a graft is an allograft, the allograft can be obtained from a living donor or a cadaveric donor. In some cases, a graft can include one or more cells to be transplanted. In some cases, a graft can include one or more tissues to be transplanted. In some cases, a graft can include a population of cells and/or cellular components not in the form of tissue or an organ (e.g., in the form of a fluid) to be transplanted. In some case, a graft can include one or more organs to be transplanted. Examples of grafts that can be used as described herein include, without limitation, stem cells (e.g., adult stem cells and hematopoietic stem cells), Islets of Langerhans (pancreas islet cells), bone marrow, blood (e.g., whole blood), a component of blood (e.g., red blood cells, white blood cells, plasma, clotting factors, and platelets), cornea, skin (e.g., a skin graft for a face), blood vessels, heart valves, bone, heart, lung, kidney, liver, pancreas, intestine, and scalp/hair. When a graft is a fluid (e.g., blood and one or more components of blood) a transplant can also be referred to as a transfusion.

In some cases, a donor mammal, a recipient mammal, and/or a graft can be assessed for the presence of one or more senescent cells. For example, a sample obtained from a mammal (e.g., a donor mammal and/or a recipient mammal) can be assessed to determine whether one or more senescent cells are present within the mammal. For example, a sample obtained from a graft (e.g., a graft to be transplanted into a recipient mammal) can be assessed to determine whether one or more senescent cells are present within the graft. As described herein, a senescent cell can produce cf-mt-DNA, and the presence of cf-mt-DNA in a sample can be used to determine that one or more senescent cells are present in the mammal from which the sample was obtained. Any appropriate method can be used to identify the presence or absence of cf-mt-DNA in a sample (e.g., a sample obtained from a donor mammal, a recipient mammal, and/or a graft to be transplanted). For example, PCR-based techniques such as real-time PCR, and/or photometric techniques can be used to identify the presence or absence of cf-mt-DNA.

When a sample from a mammal (e.g., a donor mammal and/or a recipient mammal) and/or from a graft (e.g., cells, tissues, organs, and/or a population of cells not in the form of tissue or an organ to be transplanted) is assessed for the presence of one or more senescent cells as described herein (e.g., based, at least in part, on the present of cf-mt-DNA in the sample), the sample can be any appropriate type of sample. In some cases, a sample can be a biological sample. In some cases, a sample can contain one or more biological molecules (e.g., nucleic acids such as DNA and RNA, polypeptides, carbohydrates, lipids, hormones, and/or metabolites). Examples of samples that can be assessed as described herein include, without limitation, tissue samples (e.g., bone marrow, cornea, skin, blood vessels, heart valves, bone, heart, lung, kidney, liver, pancreas, intestine, or scalp/hair samples), fluid or liquid samples (e.g., whole blood, serum, or plasma samples), and cellular samples (e.g., pancreas islet cell samples or stem cell samples such as adult stem cell or hematopoietic stem cell samples). A sample can be a fresh sample or a fixed sample (e.g., a formaldehyde-fixed sample or a formalin-fixed sample). In some cases, a sample can be a processed sample (e.g., an embedded sample such as a paraffin or optimal cutting temperature (OCT) compound embedded sample, or a sample processed to isolate or extract one or more biological molecules).

In some cases, determining that a sample (e.g., a liquid sample such as a blood sample) has an elevated level of cf-mt-DNA can indicate that the mammal contains senescent cells or an undesirably high level of senescent cells. For example, a blood sample obtained from a donor mammal that is determined to have greater than 20,000 copies of cf-mt-DNA (e.g., about 25,000 copies of cf-mt-DNA, about 30,000 copies of cf-mt-DNA, about 40,000 copies of cf-mt-DNA, about 50,000 copies of cf-mt-DNA, about 60,000 copies of cf-mt-DNA, about 70,000 copies of cf-mt-DNA, about 80,000 copies of cf-mt-DNA, about 90,000 copies of cf-mt-DNA, about 100,000 copies of cf-mt-DNA, about 120,000 copies of cf-mt-DNA, about 130,000 copies of cf-mt-DNA, about 140,000 copies of cf-mt-DNA, or about 150,000 copies of cf-mt-DNA) per mL of blood can be classified as having an undesirably high level of senescent cells. In some cases, such a donor mammal (and/or a graft from such a donor and/or the recipient of a graft from such a donor) can be treated as described herein. As another example, a blood sample obtained from a donor mammal that is determined to have less than 20,000 copies of cf-mt-DNA per mL of blood can be classified as lacking an undesirably high level of senescent cells. In some cases, such a donor mammal (and the graft from such a donor and the recipient) can be proceed with being a donor without being treated as described herein. As used herein, an elevated level of cf-mt-DNA that can indicate that the mammal contains an undesirably high level of senescent cells is any level greater than 20,000 copies of cf-mt-DNA per mL of sample (e.g., blood).

In some cases, treating a donor mammal, a graft obtained from a donor mammal, and/or a recipient mammal as described herein (e.g., by administering a composition containing one or more senotherapeutic agents, one or more TLR9 antagonists, or both one or more senotherapeutic agents and one or more TLR9 antagonists to the donor and/or recipient or by contacting a graft with a composition containing one or more senotherapeutic agents, one or more TLR9 antagonists, or both one or more senotherapeutic agents and one or more TLR9 antagonists) can be effective to reduce or maintain aging-associated phenotypes. Any appropriate method can be used to assess aging-associated phenotypes. In some cases, aging-associated phenotypes can be assessed by counting senescent cells, measuring epigenetic profiles of blood cells (“epigenetic clocks”), determining telomere length of dysfunction, and using functional tests such as for mobility, cognitive function, memory, or visual function.

In some cases, treating a donor mammal, a graft obtained from a donor mammal, and/or a recipient mammal as described herein (e.g., by administering a composition including containing one or more senotherapeutic agents, one or more TLR9 antagonists, or both one or more senotherapeutic agents and one or more TLR9 antagonists to the donor and/or recipient or by contacting a graft with a composition containing one or more senotherapeutic agents, one or more TLR9 antagonists, or both one or more senotherapeutic agents and one or more TLR9 antagonists) can be effective to reduce expression (e.g., expression and secretion) of one or more polypeptides in a senescence associated secretory phenotype (SASP). Examples of polypeptides of a SASP include, without limitation, II-6, Mcp-1, Cxcl-1, Rantes, Tnfa, Gm-csf, II-3, II-5, II-12 (p′70), II-12 (p40), II-13, Mip-1B, II-9, G-csf, Eotaxin, Ip-10, Mip-2, LIF, Cxcl-5, Vegf, activin A, Pai-1, Pai-2, and 11-8. Any appropriate method can be used to assess polypeptides of a SASP. In some cases, the presence absence, or level of a polypeptides in SASP can be assessed by RT-PCR (e.g., real-time RT-PCR), western blot, mass cytometry (CyTOF), enzyme-linked immunosorbent assay (ELISA), and mass spectroscopy.

In some cases, a composition containing one or more senotherapeutic agents can include the senotherapeutic agent(s) as the sole active ingredient for improving transplant outcomes. In some cases, a composition containing one or more one or more TLR9 antagonists can include the TLR9 antagonist(s) as the sole active ingredient for improving transplant outcomes. In some cases, a composition containing one or more senotherapeutic agents and one or more TLR9 antagonists can include the senotherapeutic agent(s) and the TLR9 antagonist(s) as the sole active ingredients for improving transplant outcomes.

In some cases, a composition containing one or more senotherapeutic agents, one or more TLR9 antagonists, or both one or more senotherapeutic agents and one or more TLR9 antagonists can include the senotherapeutic agent(s) and/or the TLR9 antagonist(s) in combination with one or more additional ingredients that be used for improving transplant outcomes. For example, a donor mammal, a graft obtained from a donor mammal, and/or a recipient mammal being treated as described herein (e.g., by administering a composition including containing one or more senotherapeutic agents, one or more TLR9 antagonists, or both one or more senotherapeutic agents and one or more TLR9 antagonists to the donor and/or recipient or by contacting a graft with a composition containing one or more senotherapeutic agents, one or more TLR9 antagonists, or both one or more senotherapeutic agents and one or more TLR9 antagonists) also can be treated with one or more additional therapeutic agents. A therapeutic agent used in combination with one or more senotherapeutic agents, one or more TLR9 antagonists, or both one or more senotherapeutic agents and one or more TLR9 antagonists described herein can be any appropriate therapeutic agent. In some cases, a therapeutic can be an immunosuppressive agent. Examples of therapeutic agents that can be used in combination with one or more senotherapeutic agents, one or more TLR9 antagonists, or both one or more senotherapeutic agents and one or more TLR9 antagonists described herein include, without limitation, rapamycin, SASP inhibitors (e.g., ruxolitinib and metformin), nicotinamides, pterostilbene, resveratrol, and α-estradiol. In some cases, a composition including one or more senotherapeutic agents, one or more TLR9 antagonists, or both one or more senotherapeutic agents and one or more TLR9 antagonists can be administered first, and the one or more additional therapeutic agents administered second, or vice versa.

In some cases, a composition containing one or more senotherapeutic agents, one or more TLR9 antagonists, or both one or more senotherapeutic agents and one or more TLR9 antagonists can be formulated into a pharmaceutically acceptable composition for administration to a donor mammal and/or a recipient mammal or for contact with a graft obtained from a donor mammal. For example, one or more senotherapeutic agents, one or more TLR9 antagonists, or both one or more senotherapeutic agents and one or more TLR9 antagonists can be formulated together with one or more pharmaceutically acceptable carriers (additives) and/or diluents. Pharmaceutically acceptable carriers, fillers, and vehicles that can be used in a pharmaceutical composition described herein include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol (PEG; e.g., PEG400), sodium carboxymethylcellulose, polyacrylates, waxes, polyethylene-polyoxypropylene-block polymers, and wool fat.

In some cases, when a composition containing one or more senotherapeutic agents, one or more TLR9 antagonists, or both one or more senotherapeutic agents and one or more TLR9 antagonists is administered to a donor mammal and/or a recipient mammal, the composition can be designed for oral or parenteral (including subcutaneous, intramuscular, intravenous, and intradermal) administration to a donor mammal and/or a recipient mammal. Compositions suitable for oral administration include, without limitation, liquids, tablets, capsules, pills, powders, gels, and granules. Compositions suitable for parenteral administration include, without limitation, aqueous and non-aqueous sterile injection solutions that can contain anti-oxidants, buffers, bacteriostats, and solutes that render the formulation isotonic with the blood of the intended recipient. In some cases, a composition containing one or more senotherapeutic agents, one or more TLR9 antagonists, or both one or more senotherapeutic agents and one or more TLR9 antagonists can be formulated for oral administration.

In some cases, when a composition containing one or more senotherapeutic agents, one or more TLR9 antagonists, or both one or more senotherapeutic agents and one or more TLR9 antagonists is placed into contact with a graft obtained from a donor mammal, the composition can be designed for ex vivo graft perfusion and/or graft preservation.

A composition containing one or more senotherapeutic agents, one or more TLR9 antagonists, or both one or more senotherapeutic agents and one or more TLR9 antagonists can be administered to a donor mammal and/or a recipient mammal in any appropriate dose(s) or placed into contact with a graft obtained from a donor mammal at any appropriate concentration. Effective doses can vary depending on the route of administration, the age and general health condition of the subject, excipient usage, the possibility of co-usage with other therapeutic treatments such as use of other agents, and the judgment of the treating physician. An effective amount of a composition containing one or more senotherapeutic agents, one or more TLR9 antagonists, or both one or more senotherapeutic agents and one or more TLR9 antagonists can be any amount that improves transplantation outcomes without producing significant toxicity to the mammal. For example, an effective amount of dasatinib (D) can be from about 1 milligrams per kilogram body weight (mg/kg) to about 20 mg/kg (e.g., about 5 mg/kg). For example, an effective amount of quercetin (Q) can be from about 10 milligrams per kilogram body weight (mg/kg) to about 200 mg/kg (e.g., about 50 mg/kg). The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the condition being treated may require an increase or decrease in the actual effective amount administered.

A composition containing one or more senotherapeutic agents, one or more TLR9 antagonists, or both one or more senotherapeutic agents and one or more TLR9 antagonists can be administered to a donor mammal and/or a recipient mammal in any appropriate frequency or placed in contact with a graft obtained from a donor mammal for any appropriate frequency. The frequency of administration or treatment can be any frequency that improves transplantation outcomes without producing significant toxicity to the mammal. For example, the frequency of administration can be from about once a day to about once a month, from about three times a day to about once a week, or from about every other day to about twice a month. In some cases, a composition containing one or more senotherapeutic agents can be administered for three consecutive days every two weeks. The frequency of administration can remain constant or can be variable during the duration of treatment. As with the effective amount, various factors can influence the actual frequency of administration used for a particular application. For example, the effective amount, duration of treatment, use of multiple treatment agents, and route of administration may require an increase or decrease in administration frequency.

A composition containing one or more senotherapeutic agents, one or more TLR9 antagonists, or both one or more senotherapeutic agents and one or more TLR9 antagonists can be administered to a donor mammal and/or a recipient mammal for any appropriate duration or placed in contact with a graft obtained from a donor mammal for any appropriate duration. An effective duration for administering or using a composition containing one or more senotherapeutic agents, one or more TLR9 antagonists, or both one or more senotherapeutic agents and one or more TLR9 antagonists can be any duration that improves transplantation outcomes without producing significant toxicity to the mammal. For example, the effective duration can vary from several days to several months or years to a lifetime. In some cases, the effective duration can range in duration from about 10 years to about a lifetime. When contacting a graft with a composition provided herein, the effective duration can be from about 30 minutes to 2 days. Multiple factors can influence the actual effective duration used for a particular treatment. For example, an effective duration can vary with the frequency of administration, effective amount, use of multiple treatment agents, and route of administration.

In certain instances, a course of treatment and transplantation outcome can be monitored. Any appropriate method can be used to monitor transplantation outcome. For example, transplantation outcome can be assessed using any appropriate methods and/or techniques, and can be assessed at different time points.

The level of toxicity, if any, can be determined by assessing a mammal's clinical signs and symptoms before and after administering a known amount of a particular composition. It is noted that the effective amount of a particular composition administered to a mammal can be adjusted according to a desired outcome as well as the mammal's response and level of toxicity.

This document also provides non-human animal models (e.g., mouse models and rat models) and methods and materials for generating non-human animal models (e.g., mouse models and rat models) having one or more accelerated aging phenotypes. For example, an animal model provided herein (e.g., a non-human animal model) can be a non-human mammal having one or more senescent cells that were transplanted into the non-human mammal to induce one or more accelerated aging phenotypes within the non-human mammal. Any appropriate non-human animal can be used in for generating a non-human animal model having one or more accelerated aging phenotypes. In some cases, a non-human animal can be a young animal (e.g., a young wild type animal). In some cases, a non-human animal can be an old animal (e.g., an old wild type animal). In some cases, a non-human animal can be a high-fat fed animal (e.g., a young high fat fed animal). In some cases, a non-human animal can be an immunodeficient animal (e.g., a SCID animal such as a SCID-beige mouse). Examples of non-human animals that can be used for generating a non-human animal model having one or more accelerated aging phenotypes include, without limitation, mice and rats. Any appropriate number of senescent cells can be transplanted in the non-human mammal. For example, about 5 million cells or less (e.g., about 5 million, about 4.5 million, about 4 million, about 3.5 million, about 3 million, about 2.5 million, about 2 million, about 1.5 million, about 1 million, about 0.5 million, or about 0.25 million cells) can be transplanted into a non-human mammal to generate a non-human animal model having one or more accelerated aging phenotypes. In some cases, when a non-human animal model is a mouse model, about 1 million cells or less can be transplanted into the mouse model. In some cases, when a non-human animal model is a rat model, about 5 million cells or less can be transplanted into the rat model. Examples of accelerated aging phenotypes include, without limitation, muscle weakness, lipodystrophy, vascular hyporeactivity, osteoporosis, memory impairment, and kidney dysfunction. In some cases, a non-human animal model (e.g., a mouse containing exogenously provided senescent cells and having one or more accelerated aging phenotypes) can be used (e.g., in a model system) to identify agents having the ability to alleviate age-related dysfunction caused by senescent cells (e.g., human senescent cells). In some cases, a non-human animal model (e.g., a mouse containing exogenously provided senescent cells and having one or more accelerated aging phenotypes) can be used to evaluate or confirm the efficacy of one or more senolytic agents and/or one or more TLR9 antagonists.

The invention will be further described in the following examples, which do not limit the scope of the invention described in the claims.

### EXAMPLES

### Example 1: Senolytics Improve Physical Function and Increase Lifespan in Old Age

Physical function declines in old age, portending disability, increased health expenditures, and mortality. Cellular senescence, leading to tissue dysfunction, may contribute to these consequences of aging. This example demonstrates that transplanting relatively small numbers of senescent cells into young mice is sufficient to cause persistent physical dysfunction, as well as to spread cellular senescence to host tissues. Transplanting even fewer senescent cells had the same effect in older recipients, accompanied by reduced survival, indicating the potency of senescent cells in shortening health- and life-span. The senolytic cocktail, dasatinib plus quercetin (D+Q), which causes selective elimination of senescent cells, decreased the number of naturally-occurring senescent cells and their secretion of frailty-related pro-inflammatory cytokines in explants of human adipose tissue. Moreover, intermittent oral administration of senolytics to both senescent cell-transplanted younger and naturally-aged mice alleviated physical dysfunction and increased post-treatment survival by 36% while reducing mortality hazard to 65%. Thus, senescent cells can cause physical dysfunction and decreased survival even in young mice, while senolytics can enhance remaining health and lifespan in old mice.

### Results

### Transplanting Small Numbers of Senescent Cells is Sufficient to Induce Physical Dysfunction in Young Mice

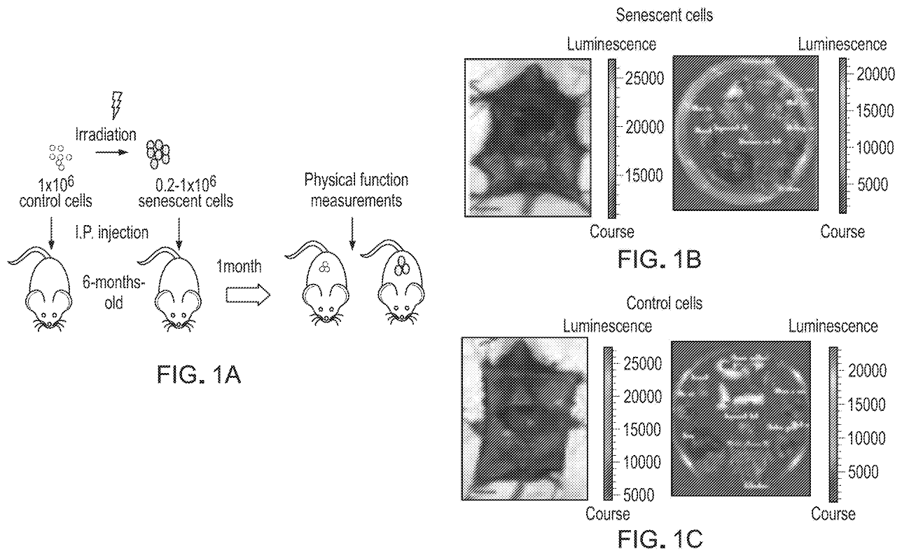

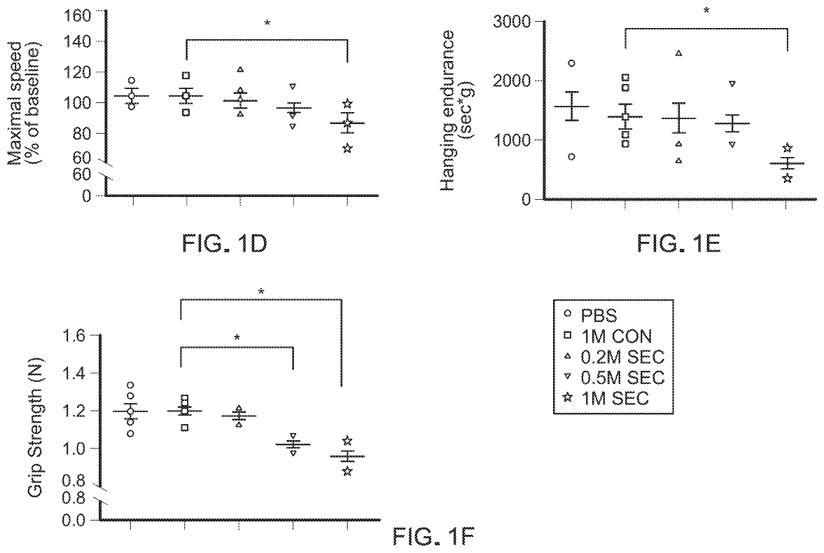

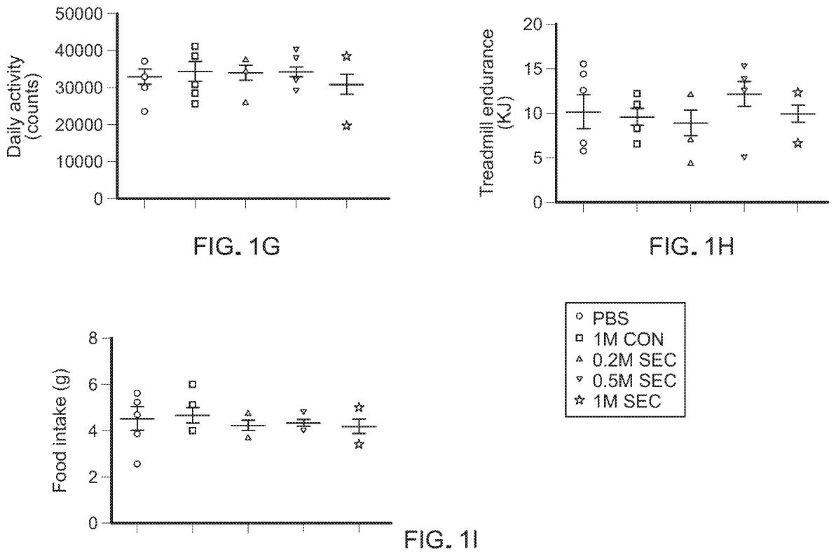

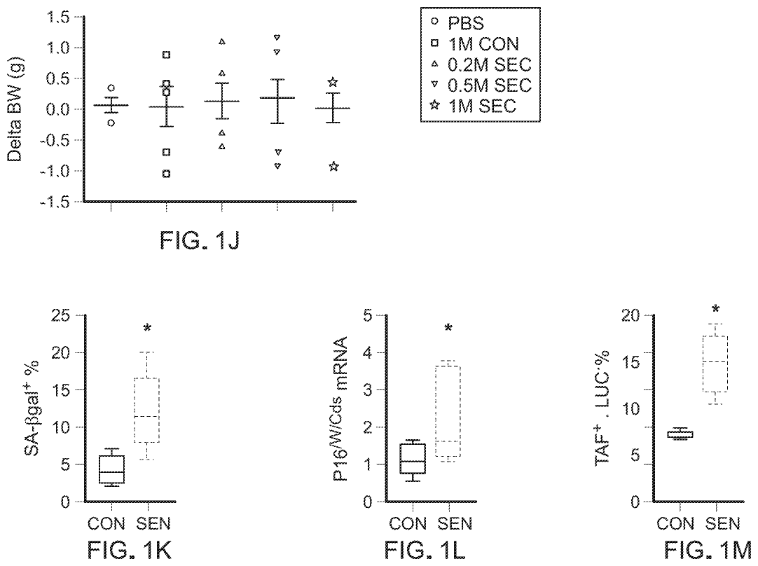

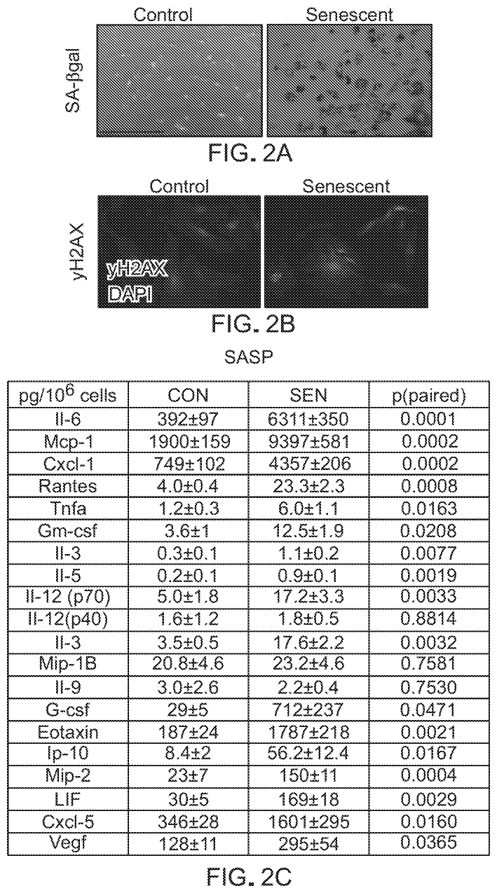

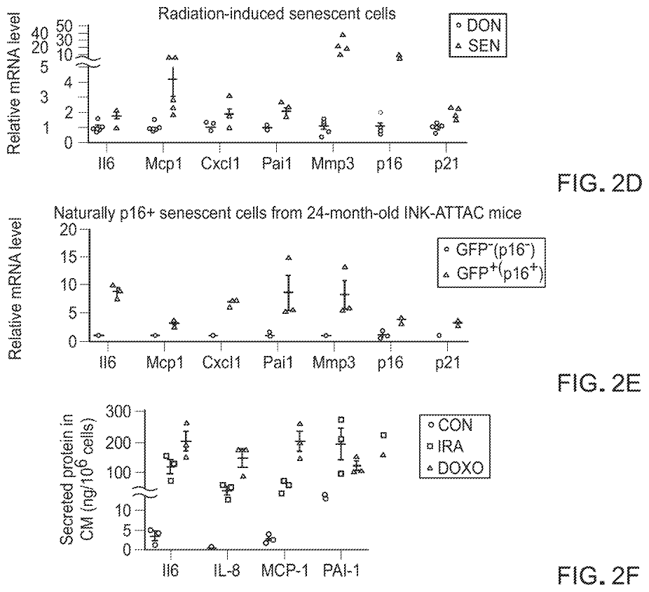

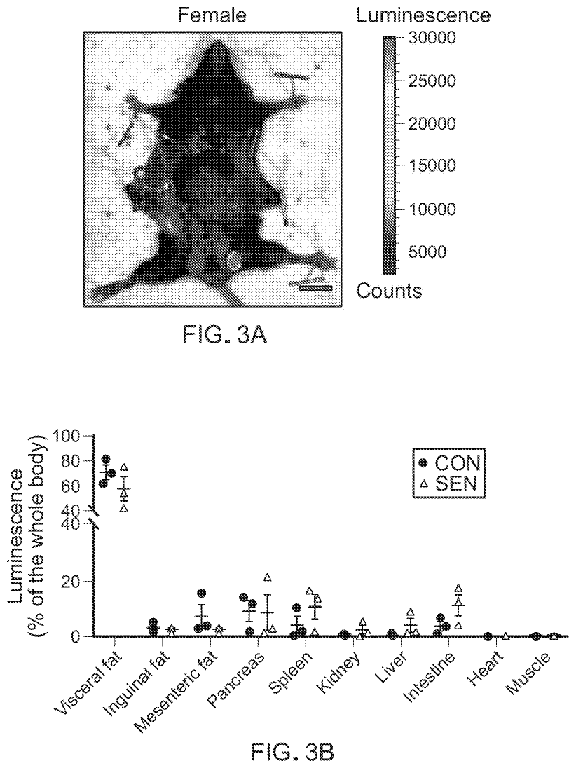

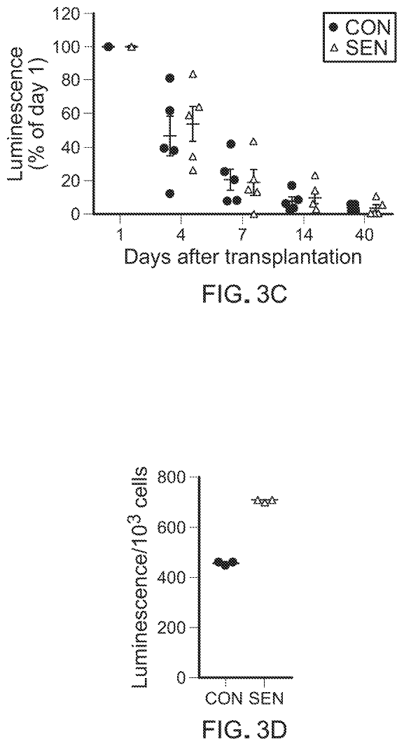

To test if senescent cells can directly cause physical dysfunction, senescent (SEN) or control, non-senescent (CON) preadipocytes (also termed adipocyte progenitors or adipose-derived stem cells) isolated from luciferase-expressing transgenic (LUC⁺) mice were transplanted intraperitoneally into syngeneic, young (6-month-old) wild type (WT) animals (FIG. 1a). Cellular senescence was induced using 10 Gray (Gy) radiation, which resulted in more than 85% of cells becoming senescent (FIG. 2a,b). Preadipocytes were transplanted because: 1) the SASP of radiation-induced senescent mouse preadipocytes (FIG. 2c,d) resembles that of endogenous senescent cells with aging (FIG. 2e) and in idiopathic pulmonary fibrosis; 2) preadipocytes are less immunogenic or subject to rejection than other cell types; and 3) these cells are arguably the most abundant type of progenitors in humans that are subject to cellular senescence. Doxorubicin was also used to induce senescence to ascertain if the physiological impact of transplanted senescent cells is limited to the context of radiation-induced senescence. Senescent cells induced by doxorubicin developed a SASP similar to that of radiation-induced senescent cells (FIG. 2f). Five days after intraperitoneal transplantation of SEN or CON preadipocytes, the cells were mainly located in visceral fat (FIG. 1b,c and FIG. 3a,b). Both the SEN and CON transplanted cells remained detectable by in vivo bioluminescence imaging (BLI) for up to 40 days (FIG. 3c). Senescent cells had higher luciferase activity than control non-senescent cells, even though they were from the same LUC transgenic mice (FIG. 3d).

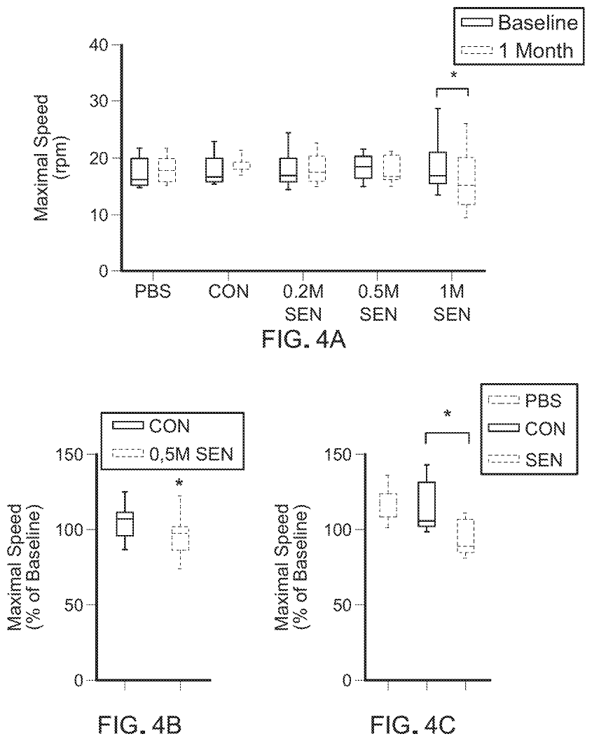

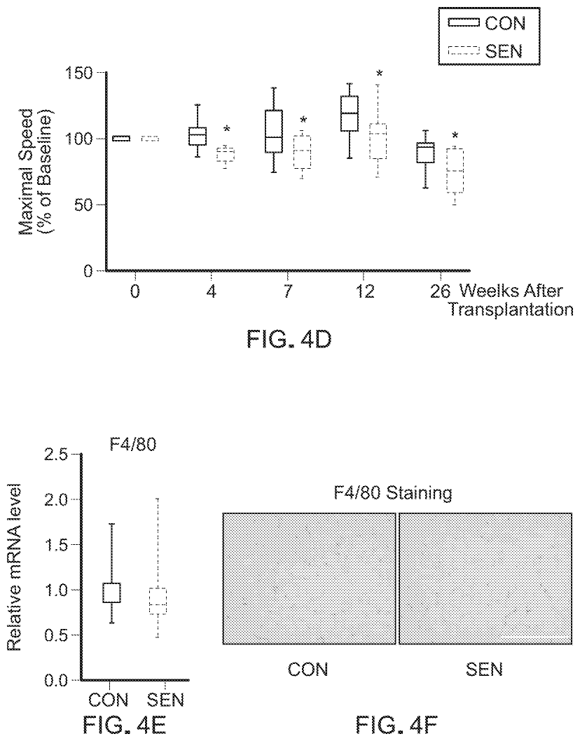

To determine whether the transplanted senescent cells induced physical dysfunction in these mice, maximal walking speed (RotaRod), muscle strength (grip strength), physical endurance (hanging test and treadmill), daily activity, food intake, and body weight were measured. Previously healthy young adult mice transplanted with 10⁶ SEN cells had significantly lower maximal walking speed, hanging endurance, and grip strength by one month after transplantation compared to mice transplanted with CON cells (FIG. 1d-f and FIG. 4a). Transplanting the same number of CON cells had no effect compared to injecting phosphate-buffered saline (PBS). Daily activity, treadmill performance, food intake and body weight were not statistically different among groups (FIG. 1g-j). Transplanting 0.5×10⁶ SEN cells was sufficient to cause decreased grip strength (FIG. 1f) and maximal walking speed (FIG. 4b), while transplanting 0.2×10⁶ senescent cells had no detectible effects. Thus, SEN cells can impair physical function in a dose-dependent manner. 6-month-old mice were estimated to have 7-8×10⁹ cells of all types, and the total cell number in intraperitoneal adipose tissue, where the transplanted cells were mainly located (FIG. 3b), is ˜3.5-6×10⁸ cells. Thus, in young mice if only 1 cell in 7,000 to 15,000 (0.01% to 0.03%) throughout the body, or 1 cell in 350 (0.28%) locally is a transplanted senescent cell, important age-related phenotypes ensue.

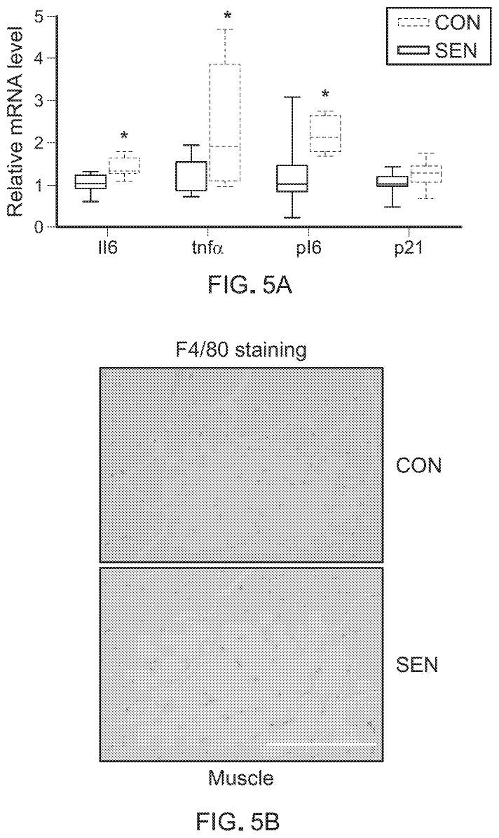

Reduced walking speed began as early as 2 weeks following a single implantation of SEN cells (FIG. 4c) and persisted for up to 6 months (FIG. 4d), yet the transplanted cells survived in vivo for only approximately 40 days. F4/80⁺ macrophage accumulation in visceral adipose tissue was not significantly increased by transplanting SEN cells (FIG. 4e,f). Therefore, if senescent cells can cause other cells to become senescent in vivo was tested by transplanting constitutively LUC-expressing SEN cells and determining whether senescence occurs in the LUC-negative recipients' tissue. Visceral fat was where most of the transplanted LUC⁺ senescent cells resided (FIG. 3b). Two months after transplantation, more senescence-associated β-galactosidase (SA-βgal)⁺ cells and higher CDKN2A (p16^{Ink4a}) expression were found in visceral adipose tissue from SEN than CON cell-transplanted mice (FIG. 1k,l), beyond the time the transplanted senescent cells were present as reflected by luciferase signal (FIG. 3c). Telomere-associated foci (TAFs; sites of DNA damage within telomeres) can also be used as a specific marker of senescence. Significantly more TAF⁺ cells were found in visceral adipose tissue of mice that had been injected with SEN than CON cells (FIG. 1m). These TAF⁺ cells were LUC⁻, so they were the recipients' own cells and not transplanted cells. F4/80⁺ macrophage accumulation was not induced in adipose tissue by the intraperitoneal SEN cell transplantation (FIG. 4e,f). Consistent with spread of senescence not only locally but also to distant tissues, expression of the markers and mediators of senescence, p16^{Ink4a}, tumor necrosis factor (TNF)α, and interleukin(IL)-6, was higher in the quadriceps muscles of SEN—than CON-transplanted mice (FIG. 5a), a tissue where transplanted cells were not detected (FIG. 3b). Similarly to adipose tissue, F4/80⁺ macrophage accumulation was not induced in muscle by SEN cell transplantation (FIG. 5b). Thus, senescence spreading may explain how a small number of transplanted senescent cells caused such profound, long-lasting, and deleterious systemic effects.

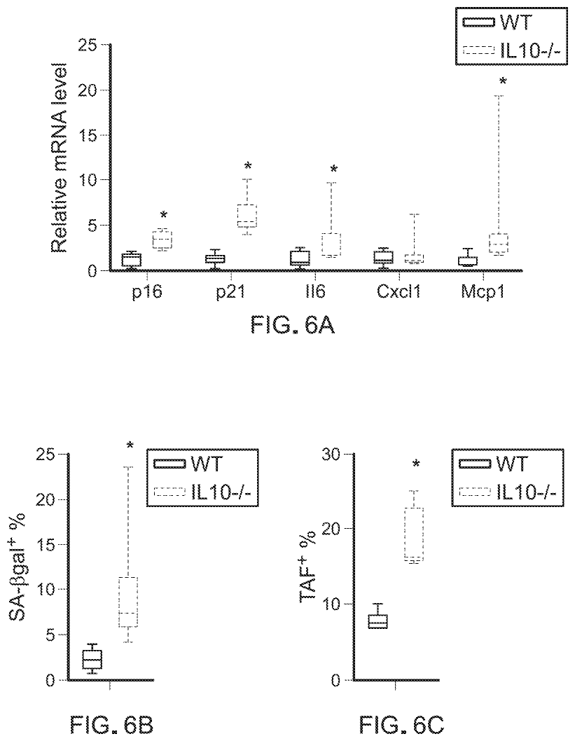

To further test whether SASP-related factors can contribute to induction of cellular senescence in vivo, IL-10 knock-out mice, which have a genetically-induced premature pro-inflammatory phenotype that resembles the SASP and which also prematurely develop physical dysfunction reminiscent of human frailty, were examined. These animals were found to have more senescent cells than wildtype controls, suggesting that the SASP can induce senescence spreading in vivo (FIG. 6a-c).

### Aging and High-Fat Diet Exacerbate Effects of Senescent Cell Transplantation

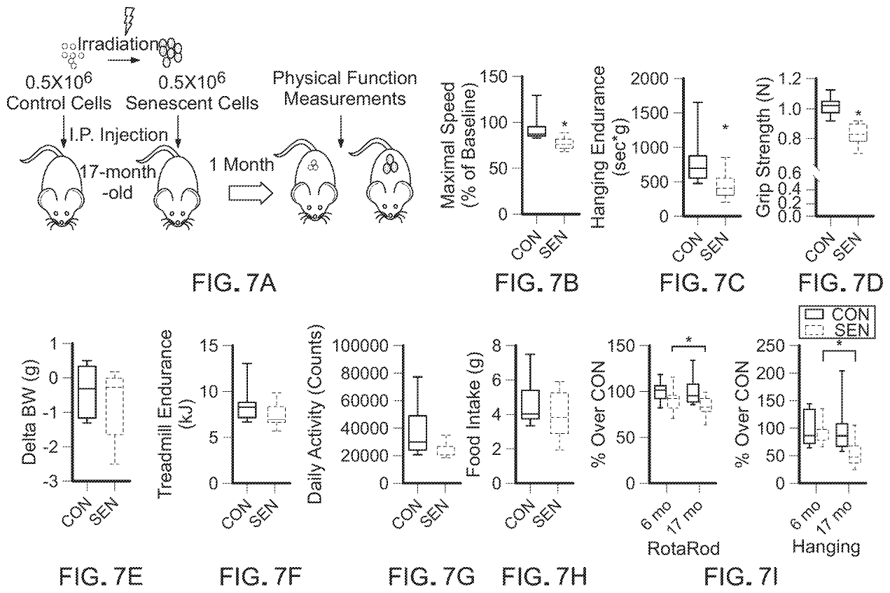

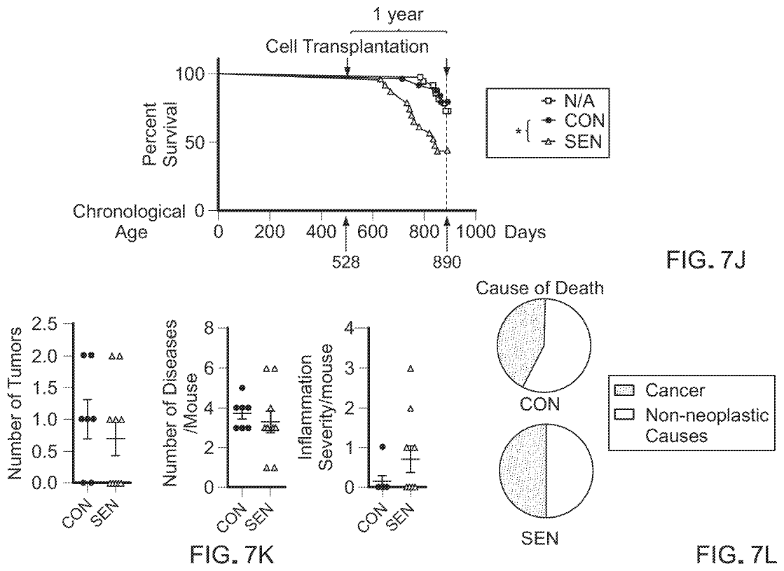

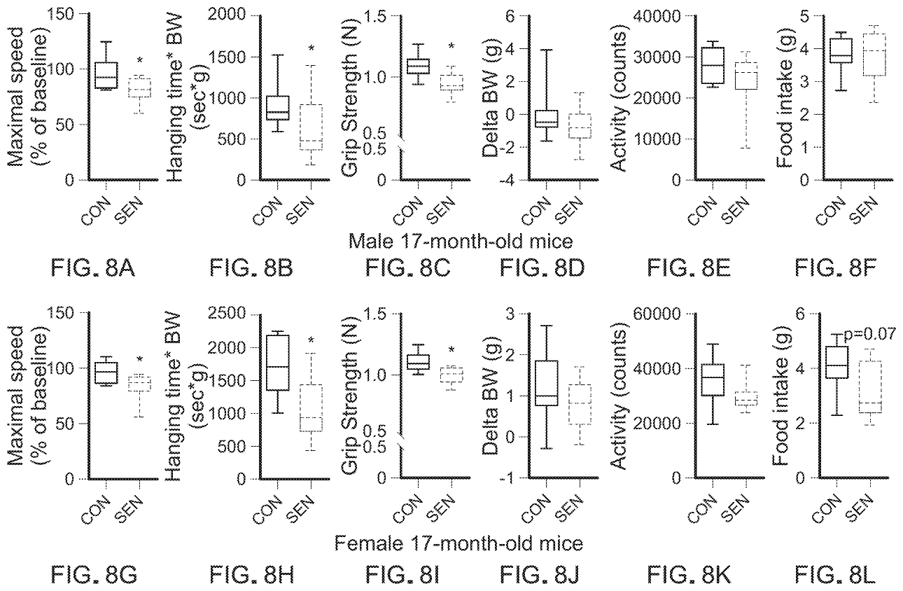

Because aging is associated with senescent cell accumulation, if increased recipient age potentiates the effects of transplanting senescent cells was tested. 0.5×10⁶ SEN or CON preadipocytes were transplanted into older (17-month) mice, so that 0.007% of all cells in the recipients were transplanted SEN or CON cells, and one month later we measured various parameters of physical function (FIG. 7a). It was found that mice transplanted with SEN cells had lower maximal walking speed, hanging endurance, and grip strength compared to CON mice (FIG. 7b-d). These findings were consistent across several independent cohorts of male (FIG. 8a-f) and female mice (FIG. 8g-l). Body weight, treadmill performance, daily activity, and food intake were not statistically different after transplanting SEN cells into the older mice (FIG. 7e-h). Transplanting 0.5×10⁶ SEN cells led to greater impairment in walking speed and hanging endurance in 17-month-old mice than 6-month-old mice (FIG. 7i), while other parameters showed no statistically significant difference. In the 17 month-old mice transplanted with SEN cells, survival for the following year was significantly lower than that of age-matched CON mice, with a 5.2 fold higher risk of death (mortality hazard ratio, P=0.006) (FIG. 7j). Tumor burden, disease burden at death, and causes of death were not significantly altered by SEN cell transplantation compared to CON cells (FIG. 7k,l), suggesting that a small number of senescent cells may shorten survival through a general process, such as hastening the progression of aging, rather than by inducing any one or a few individual diseases. Thus, augmenting senescent cell burden induces physical dysfunction, more so in middle-aged than younger individuals, and increases mortality.

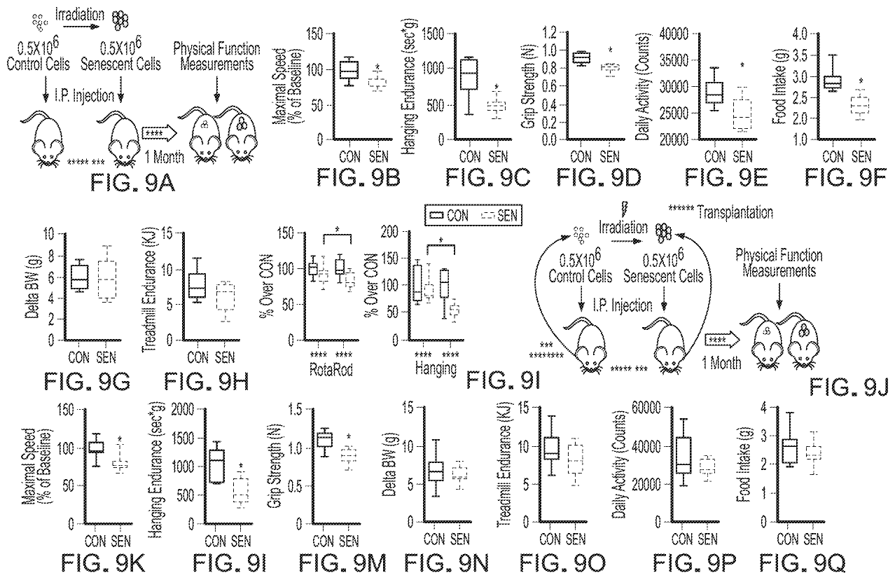

To determine whether augmenting senescent cell burden reduces resilience in the face of high fat intake, SEN or CON preadipocytes were transplanted into 8-month-old non-obese mice and then fed a high-fat diet (HFD) for one month, followed by measurements of physical function (FIG. 9a). The HFD-fed mice transplanted with 0.4×10⁶ SEN cells had lower maximal walking speed, hanging endurance, grip strength, daily activity, and food intake compared to HFD-fed mice transplanted with 0.4×10⁶ CON cells (FIG. 9b-f). Body weight and treadmill performance were not statistically different (FIG. 9g,h). SEN cell-transplanted mice on the HFD had more impairment in walking speed and hanging endurance than age-matched transplanted mice on a normal chow diet (NCD), while other parameters were not statistically significantly changed (FIG. 9i). Collectively, these data indicate that transplanting small numbers of senescent cells causes greater systemic dysfunction in older individuals or in the context of metabolic stress.

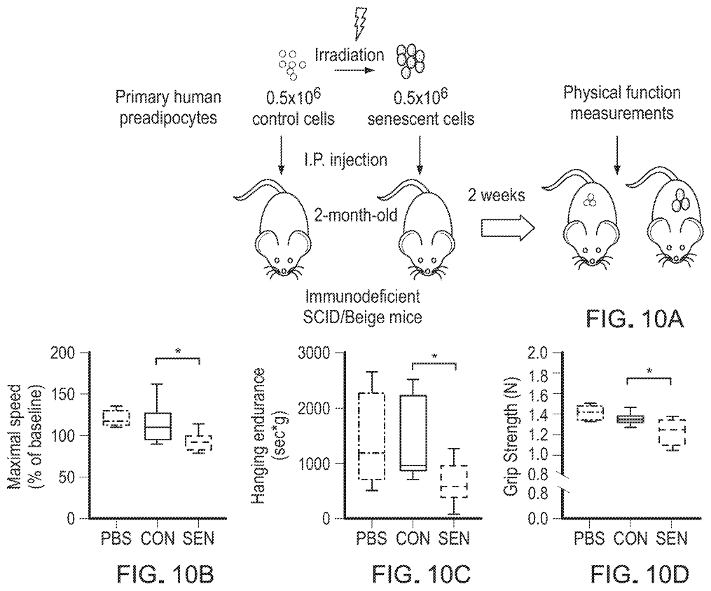

To test if the physical dysfunction in mice transplanted with SEN cells was due to transplant rejection, mice were transplanted with autologous ear fibroblasts in which senescence had been induced by 10 Gy radiation vs. sham-radiated CON cells. Senescent fibroblasts have a similar SASP to senescent preadipocytes (FIG. 2c). 10⁶ SEN or CON cells were transplanted back into the same mice from which the ear fibroblasts had been isolated and the mice were put on a HFD for 1 month (FIG. 9j). Transplanting autologous SEN ear fibroblasts intraperitoneally, compared to CON cells, led to impaired maximal walking speed, hanging endurance, and grip strength (FIG. 9k-q), as had occurred after transplanting non-autologous senescent preadipocytes. Moreover, radiation-induced SEN primary human preadipocytes vs. sham-radiated CON cells were transplanted into severe combined immunodeficiency-beige (SCID-beige) mice with impaired T cell, B cell, and natural killer cell function. Similar to the effect of mouse senescent cells on immune-competent mice, physical function was impaired in the immune-deficient mice transplanted with the human SEN cells compared to those treated with CON cells or with PBS (FIG. 10). Combined with our finding that SEN and CON cells persisted for the same amount of time after transplantation (FIG. 3c), these data support the possibility that physical dysfunction did not arise principally as a consequence of transplant immune rejection or in response to a particular type of SEN cell.

### D+Q Reduces Senescent Cell Burden and Decreases Pro-Inflammatory Cytokine Secretion in Human Adipose Tissue

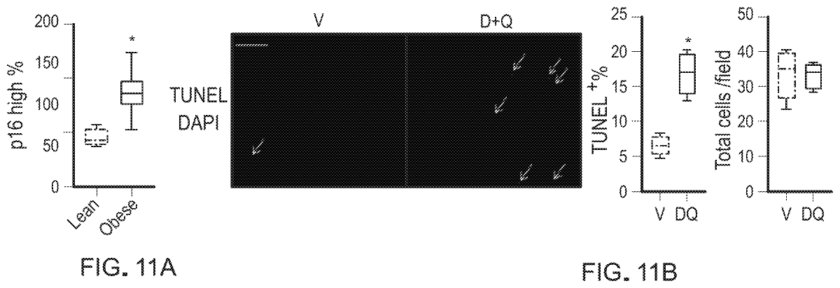

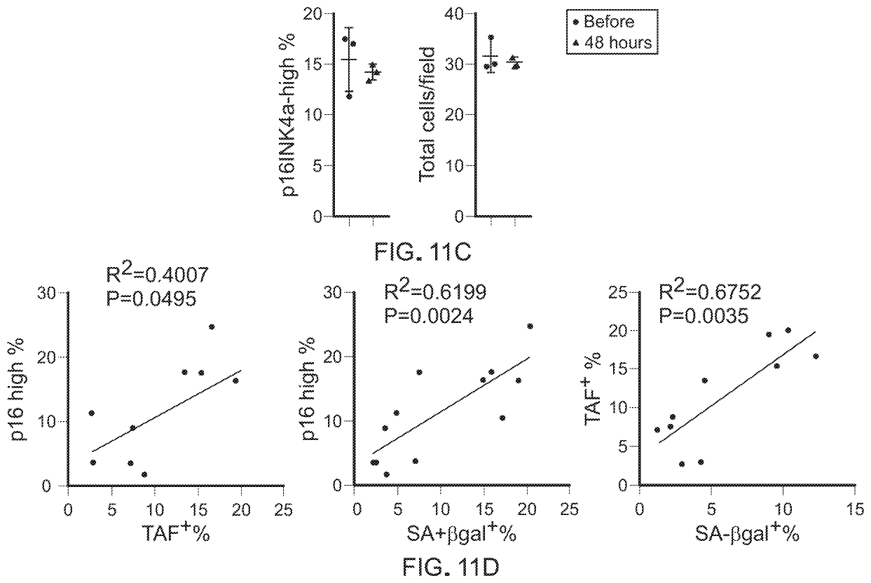

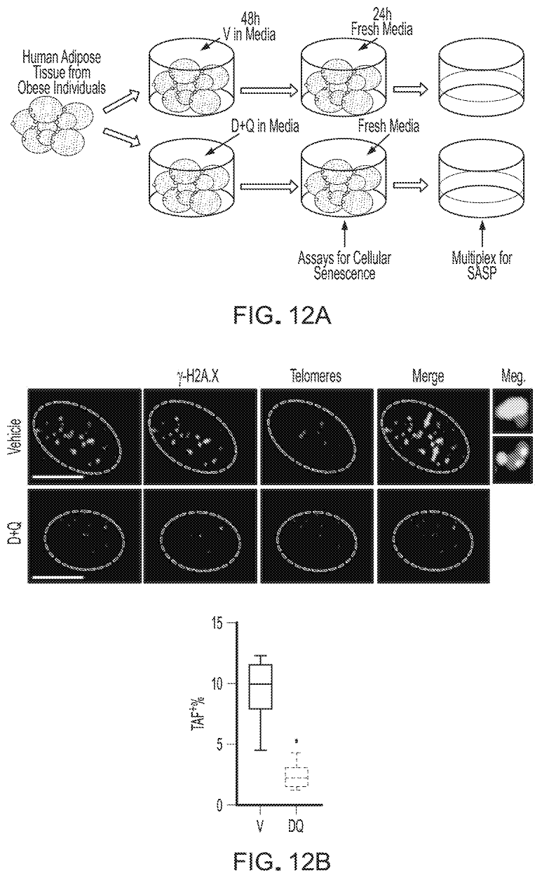

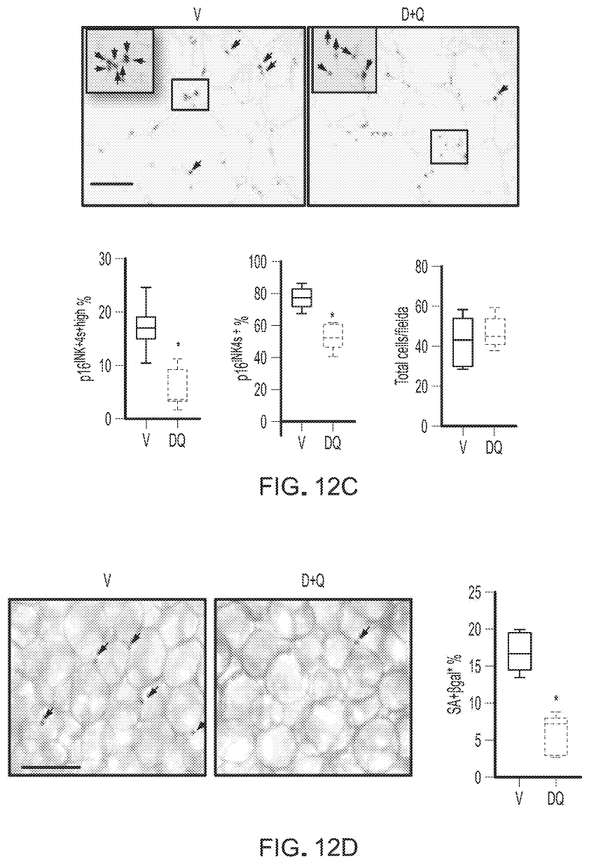

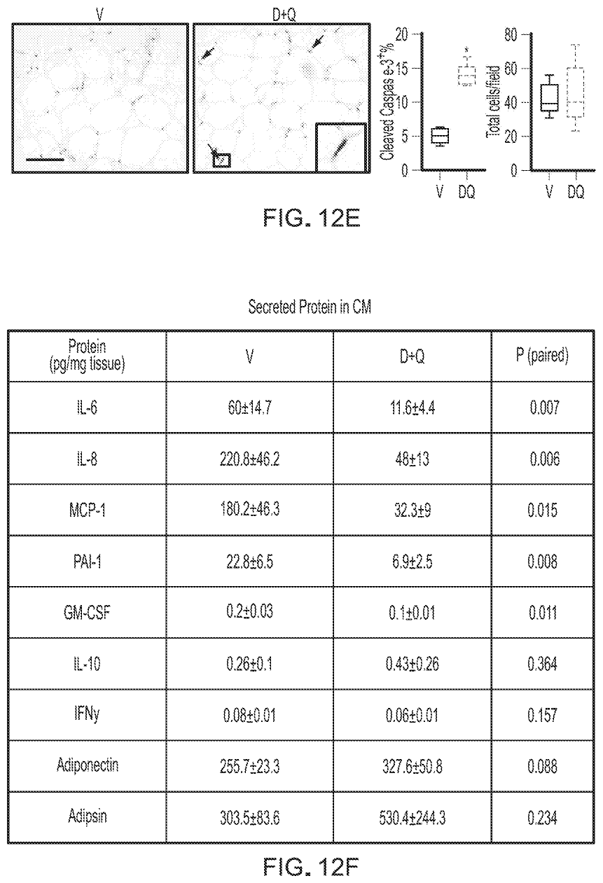

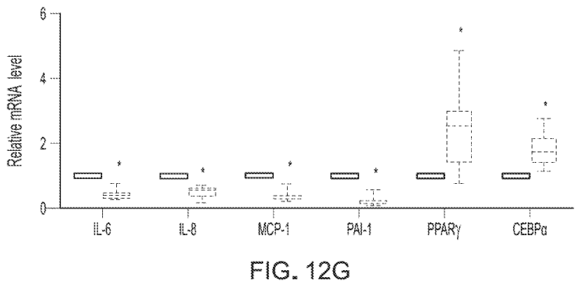

Based on the finding that senescent cells cause physical dysfunction and shorten survival, senolytic agents were evaluated for enhancing healthspan in old individuals. To begin the process of gauging the translational potential of this approach, if the combination of dasatinib plus quercetin (D+Q) is effective in human tissue was determined. Freshly-isolated human omental adipose tissue obtained from obese individuals (BMI 45.5±9.1 kg/m²; age 45.7±8.3 years) were used. The surgically excised explants, which contained naturally-occurring senescent cells (FIG. 11a), were immediately treated with D+Q (1 μM+20 μM) or vehicle (V) for 48 hours (FIG. 12a). The explants treated with D+Q had significantly less TAF⁺, p16^{INK4A}-highly expressing, and SA-βgal⁺ cells (FIG. 12b-d) and also more cells undergoing apoptosis (FIG. 12e and FIG. 11b) compared to V-treated explants from the same subjects. Forty-eight hours of treatment with vehicle did not affect total and senescent cell numbers in these adipose tissue explants (FIG. 11c). Three senescence markers, TAF, p16^{INK4A}, and SA-βgal, correlated with each other (FIG. 11d).

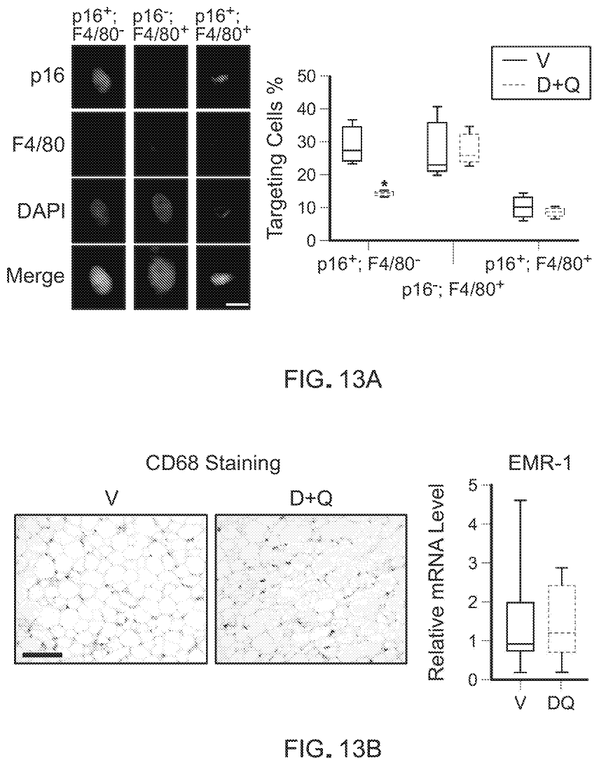

Whether D+Q had acute effects on macrophages in these explants was next examined by co-staining for p16^{INK4A} and F4/80. Only the p16^{INK4A+}; F4/80⁻ cell population was lower in the explants treated with D+Q compared to V, while the numbers of p16^{INK4A+}; F4/80⁺ or p16_{INK4A-}; F4/80⁺ cells were not affected significantly (FIG. 13a). In addition, expression of CD68 and EMR1, two macrophage markers, was not altered acutely by D+Q in the adipose tissue explants (FIG. 13b). These data suggest that D+Q has little direct effect on macrophages, including p16^{INK4A+} macrophages.

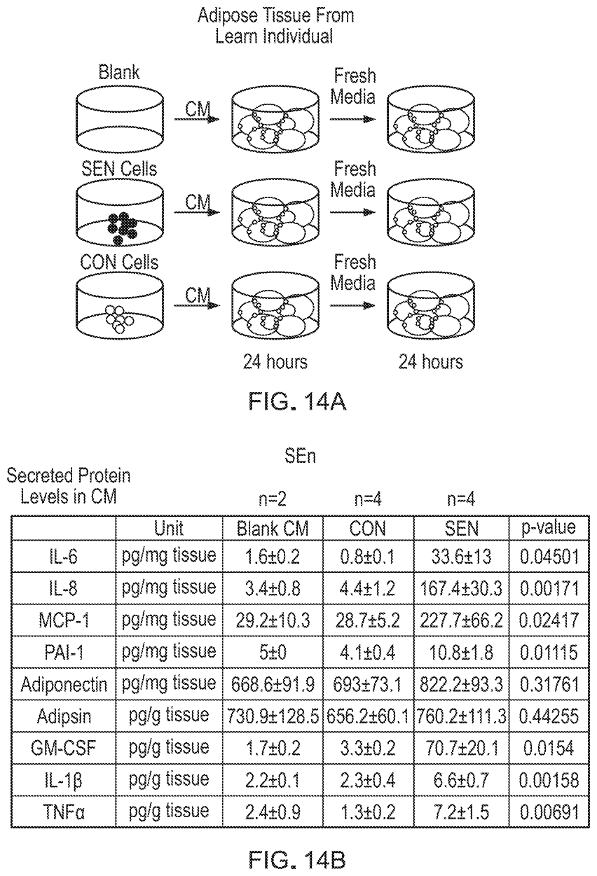

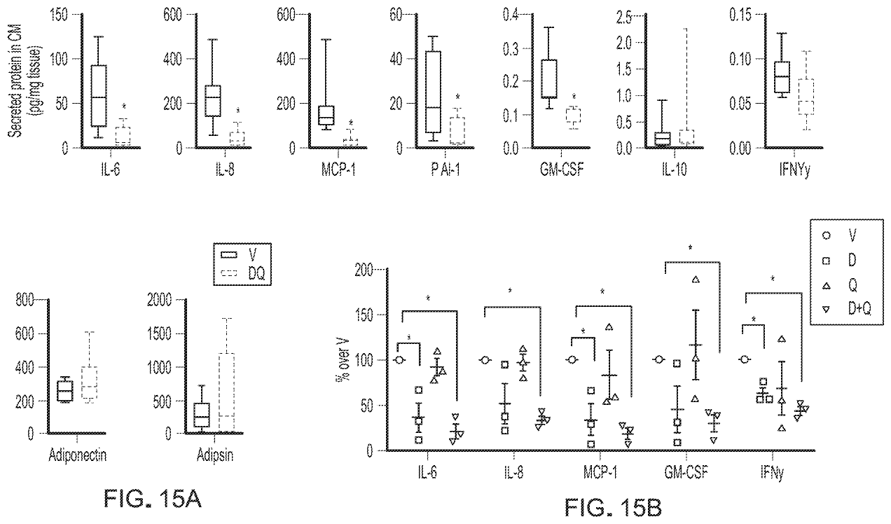

SEN preadipocytes produce a variety of pro-inflammatory cytokines and can also induce cytokine production by adipose tissue in vitro (FIG. 14), potentially leading to amplification of adipose tissue inflammation. To test if D+Q decreases cytokine secretion by adipose tissue from obese individuals, explants were treated with D+Q or V for 48 hours then washed the explants. Conditioned medium (CM) was collected in the absence of drugs over the next 24 hours. Secreted protein levels were measured in CM after D+Q treatment, and less secretion of the key SASP components, IL-6, IL-8, monocyte chemotactic protein-1 (MCP-1), plasminogen activator inhibitor-1 (PAI-1), and granulocyte macrophage colony-stimulating factor (GM-CSF) was found in CM from the D+Q group compared to V, while two non-SASP-related factors, IL-10 and interferon (IFN)-γ, were not statistically significantly altered (FIG. 12f and FIG. 15a). Furthermore, secretion of two adipokines, adiponectin and adipsin (markers of adipose tissue function), were not lower in D+Q group, excluding a non-specific effect of D+Q on protein secretion or overall cell viability (FIG. 12f). D+Q increased expression of PPARγ and CEBPα, two key transcription factors that are required for adipose tissue function through regulating adipogenesis and adipose tissue insulin responsiveness (FIG. 12g). D+Q reduced cytokine production more extensively in the human adipose tissue explants than either D or Q alone (FIG. 15b). Increases in IL-6, MCP-1, and p16^{INK4a} are associated with frailty in humans. Thus, it appears that D+Q can kill naturally-occurring human senescent cells and can attenuate secretion of inflammatory cytokines associated with human age-related frailty.

### Eliminating Senescent Cells Both Prevents and Alleviates Physical Dysfunction Induced by Senescent Cell Transplantation

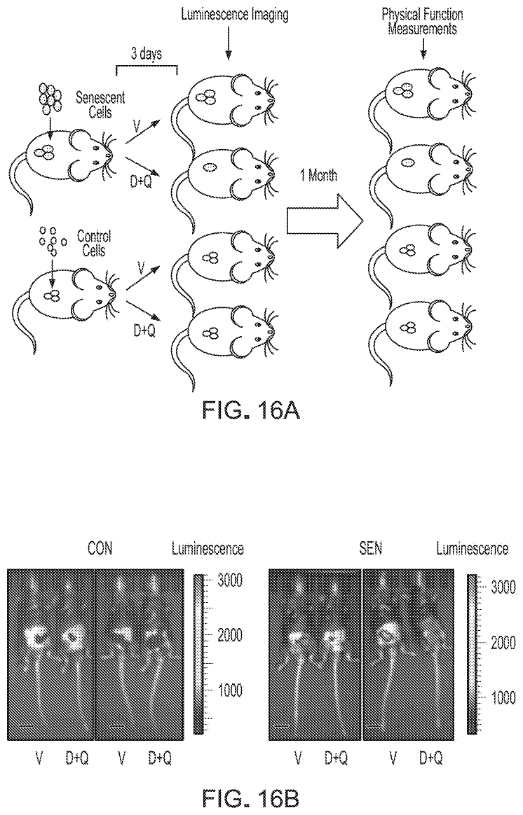

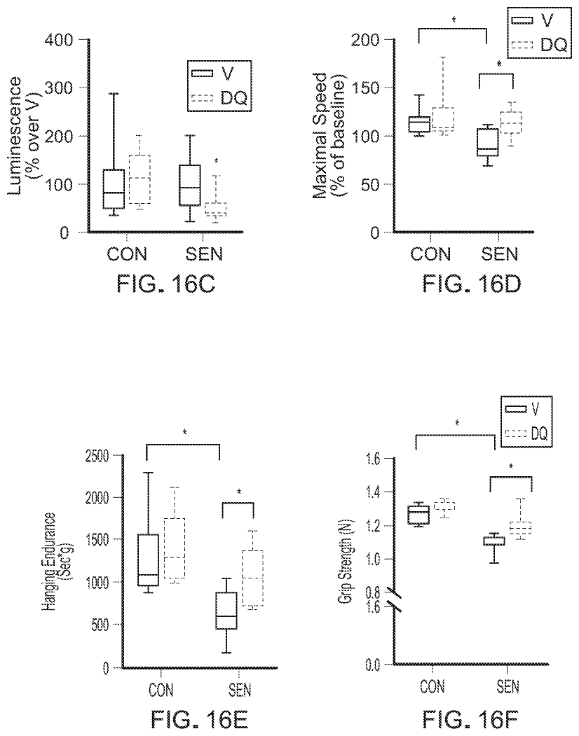

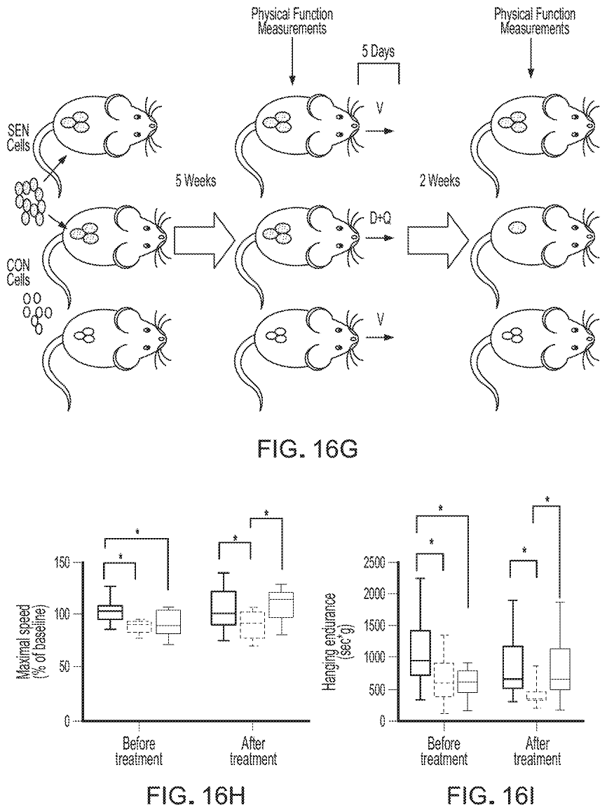

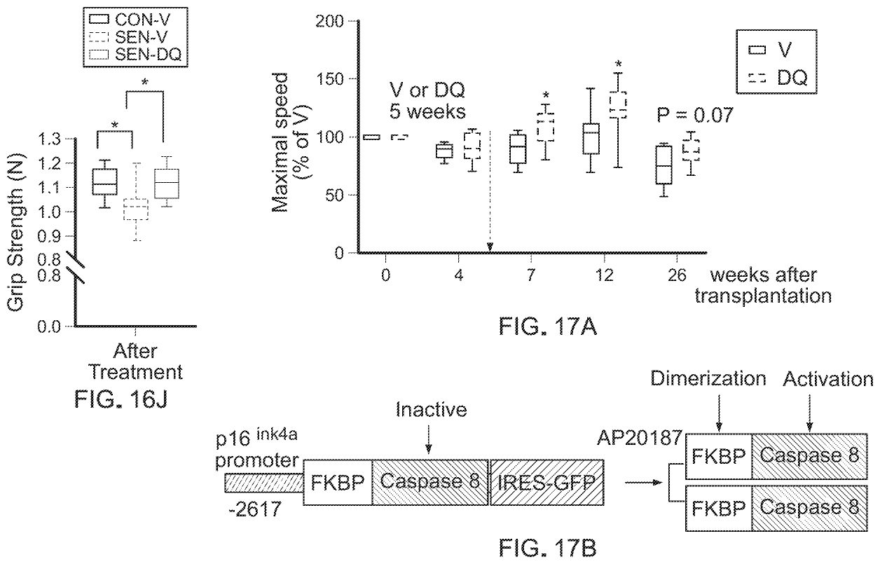

If D+Q kills transplanted senescent cells in vivo was tested by injecting SEN or CON preadipocytes that that constitutively express LUC (LUC⁺) intraperitoneally into non-luciferase-expressing WT mice. The mice were treated immediately after transplantation with D+Q or vehicle (V) for 3 days (FIG. 16a). Luminescence was significantly lower in SEN cell-transplanted mice treated with D+Q compared to V, while no difference was observed following treatment of mice transplanted with LUC⁺ CON cells (FIG. 16b,c), confirming D+Q is senolytic in vivo. These findings further support the conclusion that D+Q can selectively kill senescent cells.

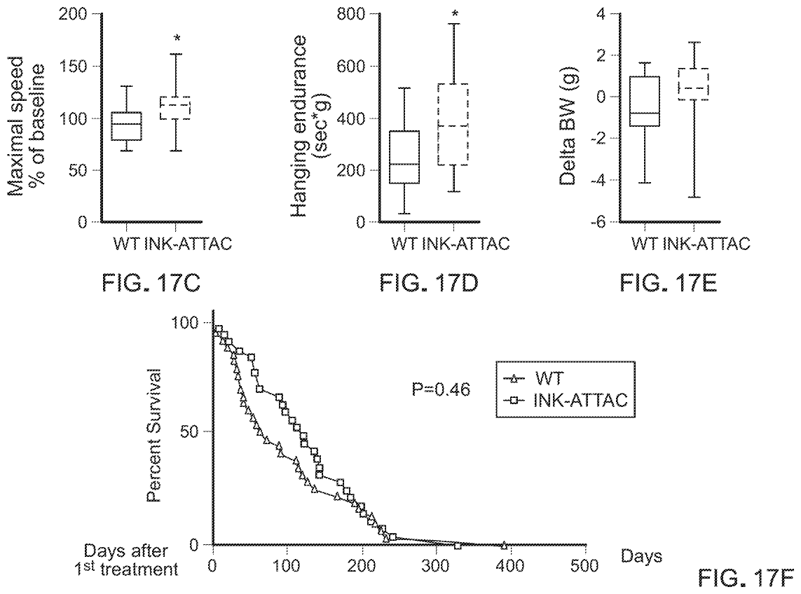

It was ascertained if clearing transplanted SEN cells using D+Q prevents development of physical dysfunction. Treating young mice with D+Q at the time of SEN cell transplantation for 3 days attenuated the deteriorations in walking speed, hanging endurance, and grip strength that occurred 1 month later in vehicle-treated SEN cell-transplanted mice, consistent with the possibility that D+Q are sufficient to prevent the physical dysfunction caused by SEN cells (FIG. 16d-f). D+Q also alleviated the physical dysfunction that occurred in mice 5 weeks following SEN cell transplantation (FIG. 16g). In the SEN cell-transplanted mice, a single course of 5 days of D+Q treatment improved physical function compared to V (FIG. 16h-j). This improvement was evident 2 weeks after D+Q treatment and lasted for several months (FIG. 17a). At these two time points of D+Q administration (immediately vs. 5 weeks after transplantation), the beneficial effects of D+Q were comparable. Therefore, the time of administration of senolytics may be flexible, potentially increasing their clinical utility. Because D+Q have elimination half-lives of <12 hours, this sustained improvement in physical function following a single course of D+Q treatment does not involve mechanisms that require continuous presence of the drugs, such as occupancy of a receptor or sustained effects on an enzyme. These findings suggest that the senolytic activity of D+Q is sufficient to attenuate senescent cell-induced physical dysfunction.

Clearance of Senescent Cells Alleviates Physical Dysfunction and Increases Late-Life Survival without Extending Morbidity in Aged Mice

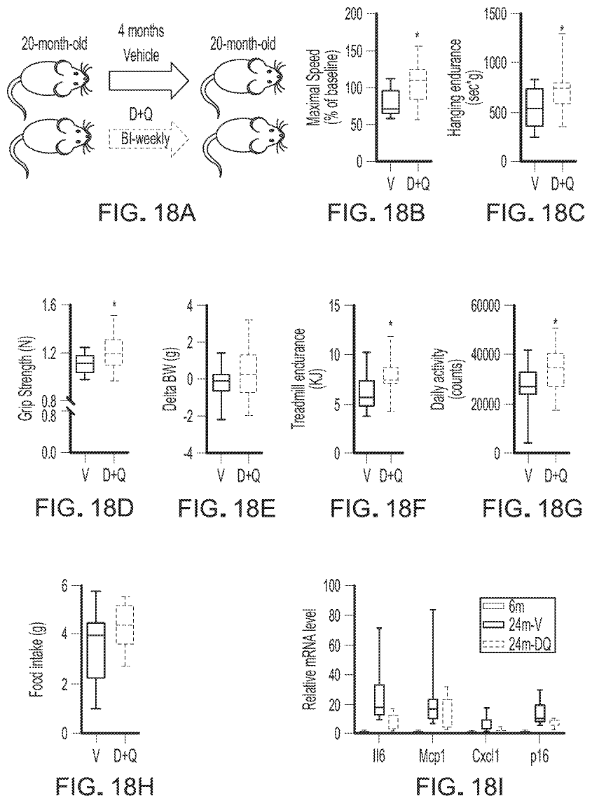

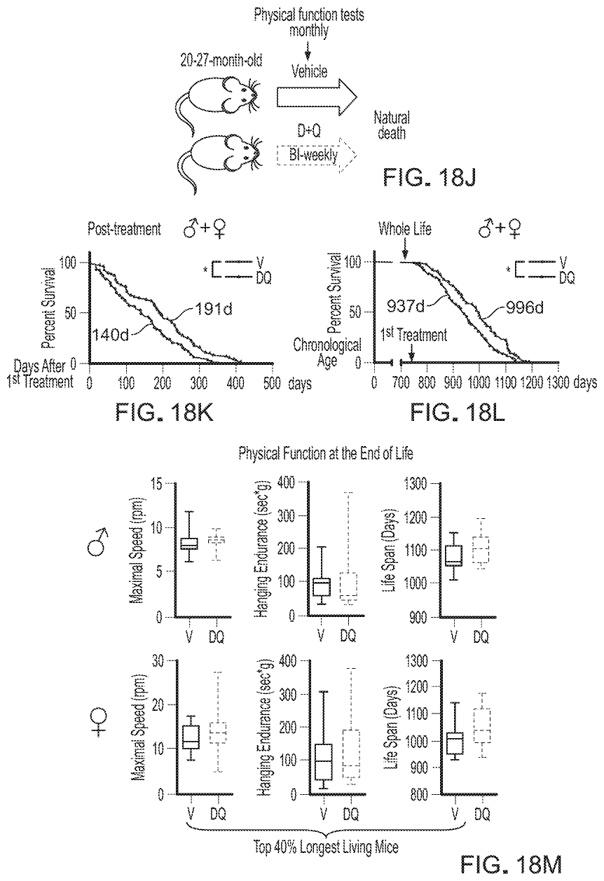

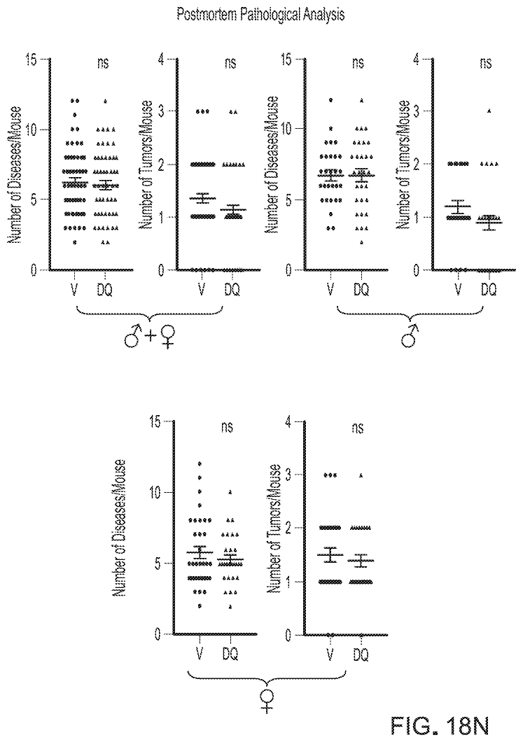

To test the role of senescent cells in physical dysfunction in aged mice, 20-month-old non-transplanted, wild-type mice were treated with D+Q or V intermittently for 4 months (FIG. 18a). D+Q alleviated physical dysfunction, with higher maximal walking speed, hanging endurance, grip strength, treadmill endurance, and daily activity in mice treated with D+Q compared to V (FIG. 18b-g). Food intake also tended to be higher in D+Q treated mice (P=0.074; FIG. 18h). Moreover, the expression of several key SASP components was lower in the visceral adipose tissue from aged mice treated with D+Q compared to V (FIG. 18i), concordant with lower secretion of SASP factors by human adipose tissue treated by D+Q (FIG. 12g).

To further test the possibility there could be a causal role for senescent cells in inducing physical dysfunction, the transgenic INK-ATTAC mouse model was used, in which endogenous p16^{Ink4a+} cells, many of which are senescent, can be genetically cleared by activating the caspase-8 moiety of ATTAC, which is expressed only in p16^{Ink4a+} cells. Consistent with the findings in mice treated with D+Q, reducing the burden of highly p16^{Ink4a}-expressing cells in 26-28 month old INK-ATTAC^{+/-} mice also alleviated physical dysfunction (FIG. 17b-f).

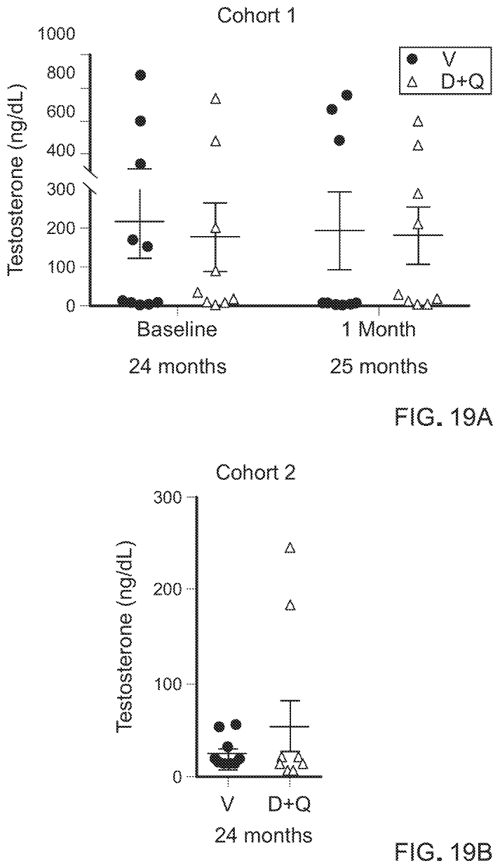

To test if testosterone levels are affected by D+Q, circulating testosterone levels were measured in two cohorts and no statistically significant differences between the V and D+Q groups were found (FIG. 19).

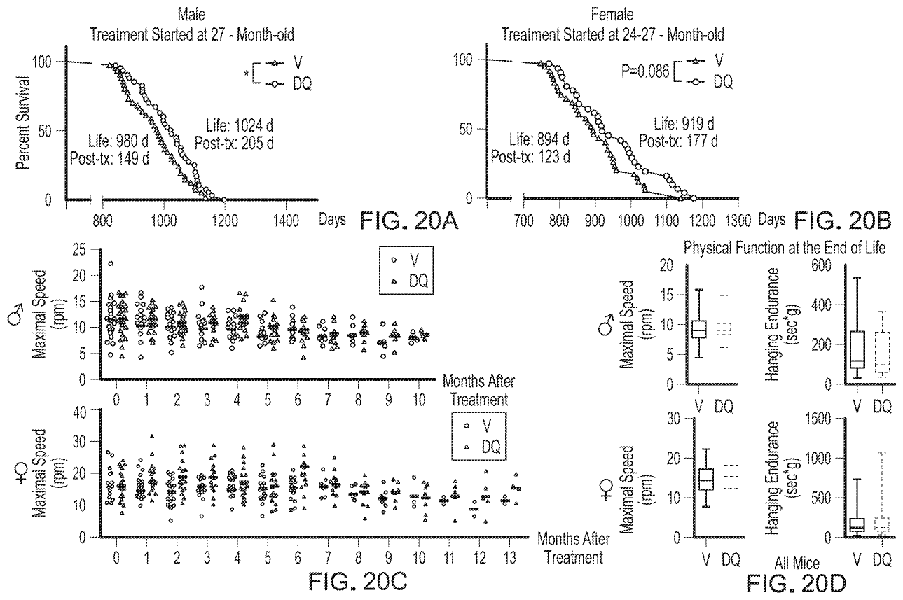

Next, it was tested if eliminating senescent cells using a potentially translatable approach, intermittent treatment with senolytics beginning at very old age, extends remaining lifespan in wild-type mice (FIG. 18j). Remarkably, mice with bi-weekly administration of D+Q starting at 24-27 months of age (equivalent to age 75-90 years in humans) had 36% higher median post-treatment lifespan and lower mortality hazard, 64.9% (P=0.01), compared to the vehicle group (FIG. 18k,l and FIG. 20a,b), indicating that senolytics can reduce risk of death in old mice.

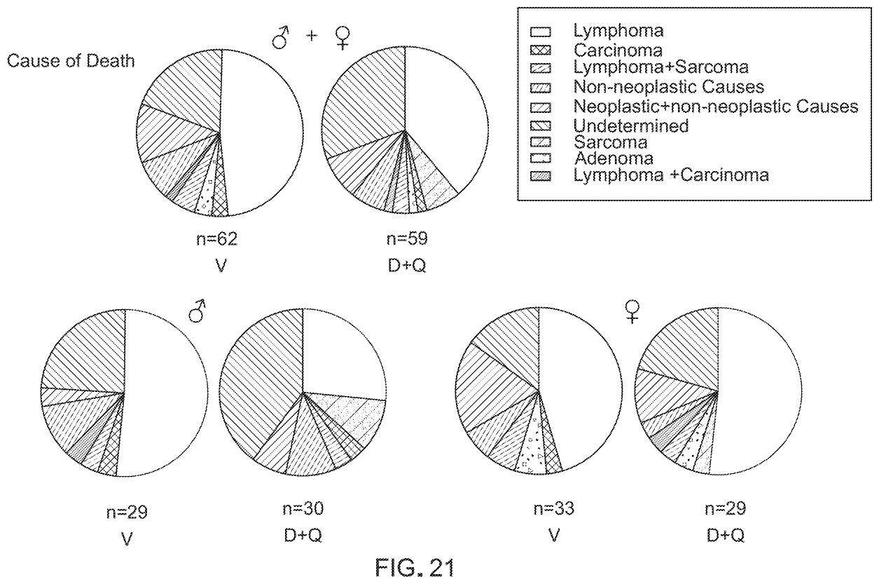

To test if this reduced mortality in old mice comes at the cost of an increased period of late-life morbidity, physical function was assessed in mice treated with D+Q or V monthly until death. Despite the longer remaining lifespan in the D+Q-treated mice, physical function in their last 2 months of life was not lower compared to V-treated mice in either males or females (FIG. 18m and FIG. 20c,d). At autopsy, the prevalence of several age-related diseases, tumor burden, and cause of death, was not statistically different between D+Q- and V-treated mice in either males or females (FIG. 18n and FIG. 21). Thus, orally-active senolytic drugs, which reduce the burden of senescent and possibly other cells that have exaggerated inflammatory cytokine production coupled with dependence on pro-survival SCAP pathways, can increase post-treatment lifespan without causing prolonged morbidity in mice, even when administered late in life.

### Methods

### Mouse Models and Drug Treatments.

All animal experiments were performed according to protocols approved by the Institutional Animal Care and Use Committee (IACUC) at Mayo Clinic. Wild-type C57BL/6 mice were obtained from the National Institute on Aging (NIA) and maintained in a pathogen-free facility at 23-24° C. under a 12 hour light, 12 hour dark regimen with free access to normal chow diet (standard mouse diet with 20% protein, 5% fat [13.2% fat by calories], and 6% fiber; Lab Diet 5053, St. Louis, Mo.) and water. Quarterly testing was negative for the following pathogens: Clostridium piliforme, Mycoplasma pulmonis, cilia-associated respiratory (CAR) bacillus, ectromelia, rotavirus (EDIM), Hantaan, K virus, lymphocytic choriomeningitis virus (LCMV), lactate dehydrogenase elevating virus (LDEV), mouse adenovirus 1 and 2, mouse cytomegalovirus (MCMV), mouse hepatitis virus (MHV), minute virus of mice (MVM), mouse parvovirus (MPV), mouse thymic virus (MTV), Polyoma, pneumonia virus of mice (PVM), REO3, Sendai virus, myocoptes, Theiler's murine encephalomyelitis virus (TMEV), Encephalitozoon cuniculi, Aspiculuris tetraptera, Radfordia/Myobia, and Syphacia obvelata. All mice were fed normal chow unless otherwise indicated. For high-fat feeding, a 60% (by calories) fat diet (D12492, irradiated; Research Diets, New Brunswick, N.J.) was used. All mice were housed in static autoclaved HEPA-ventilated microisolator cages (27×16.5×15.5 cm) with autoclaved Enrich-o'Cobs (The Andersons Incorporated) as bedding. Cages and bedding were changed bi-weekly. Cages were opened only in class II biosafety cabinets.

Luciferase transgenic C57BL/6 mice were obtained from the Jackson Laboratory (Bar Harbor, Me.; Stock No: 025854) that express firefly luciferase driven by the constitutively-active CAG promoter in most tissues. SCID beige mice (C.B.-17/IcrHsd-Prkdc^{scid}Lyst^{bg-J}) were purchased from ENVIGO (Huntingdon, Cambridgeshire, United Kingdom). INK-ATTAC mice were as described elsewhere (see, e.g., Baker et al., Nature 479:232-236 (2011)). In INK-ATTAC mice, expression of an ATTAC construct (see, e.g., Pajvani et al., Nature medicine 11:797-803 (2005)), is driven by a senescence-activated p16^{Ink4a} promotor sequence. AP20187, a drug that does not appear to affect cells lacking the ATTAC fusion protein, cross-links the mutated FKBP components of ATTAC, allowing the caspase-8 components to be activated through dimerization, leading to apoptosis of cells with high p16^{Ink4a} expression, which includes many senescent cells. These INK-ATTAC mice were then bred onto a C57BL/6 background (in the Van Deursen laboratory), genotyped (in the Kirkland laboratory), and aged to 24-27 months (in the Kirkland laboratory). For the lifespan study, AP20187 (BB homodimerizer, 10 mg/kg) was injected intraperitoneally into INK-ATTAC^{+/-} or wild-type mice daily for 3 consecutive days during each treatment course. These 3-day treatment courses were repeated every 2 weeks. INK-ATTAC^{+/-} and wildtype mice (24-27 months old) were age-matched littermates. AP20187 was purchased from Clontech (Mountain View, Calif.). For all dasatinib+quercetin (D+Q) treatments, D (5 mg/kg, drug/body weight) and Q (50 mg/kg) were administrated by oral gavage in 100-150 μL 10% PEG400. For treating 20-month-old mice, D+Q was delivered either once monthly or every 2 weeks, with essentially identical effects. For the lifespan study, 24-27-month-old mice were treated with D+Q or V for 3 consecutive days every 2 weeks. D was purchased from LC Laboratories (Woburn, Mass.). Q and doxorubicin were purchased from Sigma-Aldrich (St Louis, Mo.). All other reagents were purchased from Thermo Fisher Scientific (Waltham, Mass.) unless indicated otherwise.

### Lifespan Studies

For cell transplantation studies, 16-month-old male C57BL/6 mice were obtained from the National Institute on Aging (NIA). Mice were housed 4-5 per cage. Mice were sorted using body weight from low to high. Next, either SEN or CON transplant treatments were assigned to every other mouse using a random number generator, with the intervening mice being assigned to the other treatment, so that pairs of SEN- and CON-transplanted mice were matched by weight. After 1 month of acclimation, cells were transplanted at age 17 months. Physical function tests were performed 1 month after transplantation, at age 18 months. After that, no further tests were performed on these mice except for checking their cages. The earliest death occurred approximately 2 months after the last physical function test. For D+Q studies, 19-21-month-old C57BL/6 mice were obtained from the NIA. Mice were housed 3-5 per cage. As with the transplanted mice, animals were sorted based body weight and randomly assigned to D+Q or V treatment by a person unaware of the study design. Starting at age 24-27 months, mice were treated every 2 weeks with D+Q or V by oral gavage for 3 consecutive days. Some of the mice were moved from their original cages during the course of the study to minimize single cage-housing stress. RotaRod and hanging tests were conducted monthly because these tests are sensitive and non-invasive. Mice were euthanized and scored as having died if they exhibited more than one of the following signs: 1) unable to drink or eat; 2) reluctant to move even with stimulus; 3) rapid weight loss; 4) severe balance disorder; or 5) bleeding or ulcerated tumor. No mouse was lost due to fighting, accidental death, or dermatitis. The Cox proportional hazard model was used for survival analyses.

### Postmortem Pathological Examination.

Cages were checked every day and dead mice were removed from cages. Within 24 hours, the dead bodies were opened (abdominal cavity, thoracic cavity, and skull) and preserved in 10% formalin individually for at least 7 days. Decomposed or disrupted bodies were excluded. The preserved bodies were subjected to pathological assessment as described elsewhere (see, e.g., Ikeno et al., The journals of gerontology. Series A, Biological sciences and medical sciences 60:1510-1517 (2005)). Briefly, tumor burden (the sum of different types of tumors in each mouse), disease burden (the sum of different histopathological changes of major organs in each mouse), severity of each lesion, and inflammation (lymphocytic infiltrate) were assessed.

### Cell Culture

Mouse preadipocytes (also termed adipose-derived stem cells or adipocyte progenitors) were isolated as described elsewhere (see, e.g., Tchkonia et al., Am J Physiol Endocrinol Metab 293:E1810-1819 (2007)). Briefly, after euthanasia, inguinal fat depots were removed under sterile conditions from mice. Adipose tissue was cut into small pieces, digested in collagenase (1 mg/ml) for 60 minutes at 37° C., and then filtered through a 100 μm nylon mesh. After centrifugation at 1,000 rpm for 10 minutes, cell pellets were washed with PBS once and plated in α-MEM containing 10% FBS and antibiotics. After 12 hours, adherent preadipocytes were washed, trypsinized, and replated in order to reduce potential endothelial cell and macrophage contamination. Ear fibroblasts were isolated as described elsewhere (see, e.g., Jurk et al., Nature communications 2:4172 (2014)).

### Whole Mouse DNA Determination

All major organs and the whole skeleton from 5-month-old mice, except intestine (to exclude microbiota), were lysed in lysis buffer (100 mM Tris-HCl, pH8.8; 5 mM EDTA, pH8.0; 0.2% SDS; 200 mM NaCl; 100 μg/ml proteinase K) by rotating at 60° C. for 1 week. Most tissues except for some bone were digested. Total DNA per mouse was ˜45.5±3.6 mg (mean±SD; n=3). The molecular weight of whole mouse genome is 1.8×10¹² Daltons (Da), which is equivalent to 1.8×10¹²×1.67×10⁻²⁴=3×10¹² g. Therefore, each diploid mouse cell has at least 6×10¹² g DNA and a 5-month-old mouse has at least 7-8×10⁹ diploid cells. Using the same method, it was estimated that intraperitoneal adipose tissue from a mouse contains ˜3.5-6×10⁸ diploid cells. Based on average body weight, it was estimated that there is total of ˜7.5-8.7×10⁹ diploid cells throughout the body of a 17-month-old mouse.

### Cell Transplantation

To induce senescence in cells to be transplanted, radiation or doxorubicin, as opposed to serial passaging, was used because: 1) the radiation and chemotherapy frequently used for treating cancers can be associated with frailty (see, e.g., Ness et al., Cancer 121:1540-1547 (2015)); 2) serially-passaging mouse primary cells can induce spontaneous immortalization of mouse cells, effectively ruling out their use for transplantation (see, e.g., Xu et al., Curr Protoc Mol Biol Chapter 28, Unit 28.21 (2005)); and 3) the SASP of radiation-induced senescent preadipocytes resembles that of serial passaged senescent preadipocytes (see, e.g., Xu et al., Proc Natl Acad Sci USA 112:E6301-6310 (2015)). Senescence was induced by 10 Gy of cesium radiation or 0.2 mM doxorubicin for 24 hours as described elsewhere (see, e.g., Xu, et al., Proc Natl Acad Sci USA 112:E6301-6310 (2015); and Xu et al., Elife 4:e12997 (2015)). Radiation-induced senescent cells were used in these studies unless indicated otherwise. These cells were considered to be senescent 20 days after these treatments. More than 85% of cells were senescent based on two assays. Senescent or control cells were collected by trypsinization. Cell pellets were washed with PBS once and re-suspended in PBS for transplantation. Mice were anesthetized using isofluorane and cells were injected intraperitoneally in 150 μl PBS through a 22G needle. Injection using 22G needles was observed to not interfere with the viability of senescent or control cells.

### Bioluminescence Imaging

Mice were injected intraperitoneally with 3 mg d-luciferin (Gold Biotechnology, St. Louis, Mo.) in 200 μl PBS. Mice were anesthetized using isofluorane and bioluminescence images were acquired using a Xenogen Ivis 200 System (Caliper Life Sciences, Hopkinton, Mass.) according to the manufacturer's instructions.

### Physical Function Measurements

All measurements were performed at least 5 days after the last dose of D+Q treatment. Maximal walking speed was assessed using an accelerating RotaRod system (TSE system, Chesterfield, Mo.). Mice were trained on the RotaRod for 3 days at speeds of 4, 6, and 8 rpm for 200 seconds on days 1, 2, and 3. On the test day, mice were placed onto the RotaRod and then started at 4 rpm. The rotating speed was accelerated from 4 to 40 rpm over a 5 minute interval. The speed was recorded when the mouse dropped off the RotaRod. Results were averaged from 3-4 trials and normalized to baseline speed. Training was not repeated for mice that had been trained within the preceding 2 months. Forelimb grip strength (N−F/kg) was determined using a Grip Strength Meter (Columbus Instruments, Columbus, Ohio). Results were averaged over 10 trials. For the hanging test, mice were placed onto a 2 mm thick metal wire, 35 cm above a padded surface. Mice were allowed to grab the wire with their forelimbs only. Hanging time was normalized to body weight as hanging duration (sec)×body weight (g). Results were averaged from 2-3 trials for each mouse. A Comprehensive Laboratory Animal Monitoring System (CLAMS) was used to monitor daily activity and food intake over a 24-hour period (12 hours light and 12 hours dark). The CLAMS system is equipped with an Oxymax Open Circuit calorimeter System (Columbus Instruments). For treadmill performance, mice were acclimated to a motorized treadmill at an incline of 5° (Columbus Instruments) over 3 days for 5 minutes each day starting at a speed of 5 m/minute for 2 minutes, 7 m/minute for 2 minutes, and then 9 m/minute for 1 minute. On the test day, mice ran on the treadmill at an initial speed of 5 m/minute for 2 minutes and then the speed was increased by 2 m/minute every 2 minutes until the mice were exhausted. Exhaustion was defined as the inability to return onto the treadmill despite a mild electrical shock stimulus and mechanical prodding. Distance was recorded and total work (kJ) was calculated using the following formula: mass (kg)×g (9.8 m/s²)×distance (m)×sin(5°).

### Real-Time PCR

Trizol was used to extract RNA from tissues. RNA was reverse-transcribed to cDNA using a M-MLV Reverse Transcriptase kit (Thermo Fisher Scientific) following the manufacturer's instructions. TaqMan fast advanced master mix (Thermo Fisher Scientific) was used for real-time PCR. TATA-binding protein (TBP) was used as an internal control. Probes and primers (TBP, Mm01277042 ml; IL-6, Mm00446191 ml; TNF-α, Mm00443260_g1; p16^{Ink4a}, Mm00494449_m1; p21^{Cip1}, Mm04205640_g1) were purchased from Thermo Fisher.

### Testosterone Assay

Circulating testosterone was assayed by liquid chromatography-tandem mass spectrometry (LC-MS/MS) (Agilent Technologies, Santa Clara, Calif. 95051). Intra-assay coefficients of variation (C.V.) are 7.4%, 6.1%, 9.0%, 2.3%, and 0.9% at 0.65, 4.3, 48, 118, and 832 ng/dL, respectively. Inter-assay C.V.'s are 8.9%, 6.9%, 4.0%, 3.6%, and 3.5% at 0.69, 4.3, 45, 117, and 841 ng/dL, respectively.

### Human Adipose Tissue Explants

The protocol was approved by the Mayo Clinic Foundation Institutional Review Board for Human Research. Informed consent was obtained from all subjects. Human adipose tissue was resected during gastric bypass surgery from 8 obese subjects. Two of the subjects were male and 6 were female. Ages of subjects were 45.8±8.2 years (means±SD; range 36-58). Mean body mass index (BMI) was 45.5±9.1 kg/m² (means±SD; range 38-66). No subject was known to have a malignancy. Greater omental adipose tissue was obtained from each subject. Adipose tissue was cut into small pieces and washed with PBS 3 times. Adipose tissue was then cultured in medium containing 1 mM sodium pyruvate, 2 mM glutamine, MEM vitamins, MEM non-essential amino acids, and antibiotics with 20 μM Q and 1 μM D or DMSO. After 48 hours, the adipose explants were washed 3 times with PBS. Aliquots of adipose tissue were fixed for immunostaining or SA-βgal assay. The rest of the tissue was maintained in the same medium without drugs for 24 hours to collect conditioned medium (CM) for multiplex protein analysis. For FIG. 14, CM was collected from senescent (SEN) human primary preadipocytes, non-senescent control (CON) preadipocytes, and blank culture flasks containing no cells (Blank CM). Senescent and control preadipocytes were from the same donor. Human subcutaneous adipose tissue explants were obtained from a lean kidney donor (BMI 26.5 kg/m2; age 43 years) and divided into pieces. These explants were incubated with SEN, CON, or Blank CM for 24 hours. Next, these explants were washed with PBS, and then incubated with fresh medium for conditioning for another 24 hours.

### Multiplex Protein Analyses

Pro-inflammatory cytokine and chemokine protein levels in CM were measured using Luminex xMAP technology. The multiplexing analysis was performed using the Luminex™ 100 system (Luminex, Austin, Tex.) by Eve Technologies Corp. (Calgary, Alberta, Canada). Human multiplex kits were from Millipore (Billerica, Mass.).

### SA-βgal Assay and Immunostaining

Adipose tissue cellular SA-βgal activity was assayed as described elsewhere (see, e.g., Xu et al., Proc Natl Acad Sci USA 112:E6301-6310 (2015)). TAF immunofluorescence in situ hybridization was performed as described elsewhere (see, e.g., Hewitt et al., Nature communications 3:708 (2012)). Briefly, formalin-fixed, paraffin-embedded (FFPE) adipose tissue blocks were cut into 5 μm sections. Sections were de-paraffinized with Histoclear (National Diagnostics, Charlotte, N.C.) and hydrated using an ethanol gradient. Antigen was retrieved by incubation in 0.01 M, pH 6.0 citrate buffer at 95° C. for 10 minutes. Slides were placed into blocking buffer (goat serum or horse serum 1:60 in 0.1% BSA in PBS) for 60 minutes at room temperature. Samples were further blocked with Avidin/Biotin (Vector Lab, Burlingame, Calif.) for 15 minutes at room temperature. Primary antibody (anti-γH2AX 1:200, Cell Signaling, Danvers, Mass., #9718) was applied overnight at 4° C. in the blocking buffer. Washed slides were then incubated for 30 minutes with biotinylated, anti-rabbit secondary antibody (Vector Lab). Finally, Fluorescein Avidin DCS (Vector Lab) was applied for 20 minutes. For luciferase co-staining, a 2^{nd} primary antibody (anti-luciferase 1:500, Novusbio, Littleton, Colo., #NB100-167755) was applied overnight at 4° C. in blocking buffer. Next, washed slides were incubated with secondary antibody (Alexa Fluor647 1:500, Thermo Fisher Scientific, #A-21447). Telomeres were then stained by fluorescent in situ hybridization (FISH). Slides were fixed with 4% paraformaldehyde for 20 minutes, dehydrated in an alcohol gradient cascade, and denatured for 10 minutes at 80° C. in hybridization buffer (70% formamide [Sigma-Aldrich], 25 mM MgCl₂, 1 M Tris pH 7.2, 5% blocking reagent [Roche, Basel, Switzerland]) with a 2.5 μg/ml Cy-3-labelled telomere-specific (CCCTAA) peptide nucleic acid probe (Cat #F1002, Panagene, Daejeon, Korea) for 2 hours at room temperature in the dark. After washing, sections were incubated with 4,6-diamidino-2-henylindole (DAPI), mounted, and imaged. In-depth Z-stacking (a minimum of 20 optical slices with 100× oil objective) was used for imaging. The images were further processed using Huygens (SVI) deconvolution. Immunohistochemical (IHC) staining was performed by the Pathology Research Core (Mayo Clinic, Rochester, Minn.) using a Leica Bond RX stainer (Leica, Buffalo Grove, Ill.). Slides were retrieved for 20 minutes using Epitope Retrieval 1 (Citrate; Leica) and incubated in Protein Block (Dako, Agilent, Santa Clara, Calif.) for 5 minutes. Primary antibodies were diluted in Background Reducing Diluent (Dako) as follows: p16^{INK4a} (rabbit, monoclonal; Abcam, Cambridge, Mass.) at 1:600, cleaved-caspase 3 (rabbit, polyclonal; Cell Signaling) at 1:200, and F4/80 (rat, monoclonal; Abcam) at 1:500, except for CD68 antibody (mouse, monoclonal; Dako), which was diluted in Bond Diluent (Leica) at 1:200. All primary antibodies were diluted in Background Reducing Diluent (Dako) and incubated for 15 minutes. The detection system used was a Polymer Refine Detection System (Leica). This system includes the hydrogen peroxidase block, post primary and polymer reagent, DAB, and hematoxylin. Immunostaining visualization was achieved by incubating slides 10 minutes in DAB and DAB buffer (1:19 mixture) from the Bond Polymer Refine Detection System. Slides were counterstained for 5 min using Schmidt hematoxylin, followed by several rinses in 1× Bond wash buffer and distilled water. Slides were dehydrated using increasing concentrations of ethyl alcohol and cleared by 3 changes of xylene prior to permanent cover-slipping in xylene-based medium.

### Statistical Analysis

GraphPad Prism 7.0 was used for most statistical analyses. Two-tailed Student's t-tests were used to estimate statistically significant differences between two groups. One-way analysis of variance (ANOVA) with Tukey's post-hoc comparison was used for multiple comparisons. Pearson's correlation coefficients were used to test correlations. As mice were obtained in several cohorts and grouped in cages, the Cox proportional hazard model was used for survival analyses. The model incorporated sex and age of treatment as fixed effects, and cohorts and initial cage assignment as random effects. Due to the fact that some of mice were moved from their initial cages during the study to minimize single cage housing stress, we also conducted analyses without cage effect. Results between these two analyses did not differ substantially in directionality or statistical significance, strengthening confidence in our results. Survival analysis was performed using statistical software R (version 3.4.1; library “coxme”). Investigators were blinded to allocation during most of experiments and outcome assessments. Baseline body weight was used to assign animals to experimental groups (to achieve similar body weight between groups), so only randomization within groups matched by body weight was conducted. The sample size was determined based on previous experiments, so no statistical power analysis was used. Values are presented as mean±SEM unless otherwise indicated, with P<0.05 considered to be significant. All replicates in this study were independent biological replicates, which came from different biological samples.

### Conclusion

These results demonstrate that targeting senescent cells can improve both health- and life-span in mice. Due to the role of senescent cells in inducing physical dysfunction in mice demonstrated here, senolytics can be effective in alleviating physical dysfunction and resulting loss of independence in older human subjects.

### Example 2: Senolytics Improve Survival Following Transplant

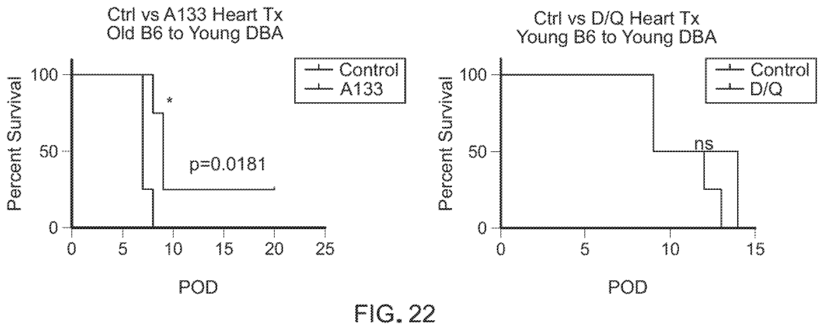

Treating young mice transplanted with hearts from old mice significantly increases survival (FIG. 22). Hearts from old C57BL/6 mice were transplanted into young DBA/2J mice w/o immunosuppression. Eight- to 12-week-old wild-type (WT) male DBA/2J WT male mice were purchased from Charles River Laboratories (Wilmington, Mass.). Eighteen-month-old WT male C57BL/6 mice were purchased from the National Institute of Aging (NIA, Bethesda, Md.). Using a modified cuff technique, fully vascularized cardiac grafts from either old or young donor mice were heterotopically transplanted into young recipients. Hearts were anastomosed to the recipient's common carotid artery and internal jugular vein. Transplantation into the recipient's cervical region facilitated reliable functional assessment through palpation. Ischemic times were kept consistently at 40 minutes with an anastomosis time of 12 minutes. Graft function was measured daily by palpation, and allograft rejection was defined as the complete cessation of palpable contractility. Graft survival is shown as the median survival time in days.

### Example 3: Enhancing Rejuvenation and Accelerating Aging Processes

In settings where young and old tissues cohabitate, donor cells and recipient environment may have bi-directional effects leading to either rejuvenation or accelerated aging. In this example, aging parabiosis studies and transplant research are juxtaposed. Parabiosis pathways studies may influence organ, tissue, and cell transplantation. Thus, studies refine the efficacy of transplant biology when using old or young organs and discuss specific mechanisms and pathways of relevance. Insights from aging research can inform and accelerate the understanding of transplant biology. Similarly, insights from a wealth of transplant studies may modify thinking in the aging biology field.

### Mechanisms of Transferring Proclivity to Aging Processes

### Factor-Mediated Contributions

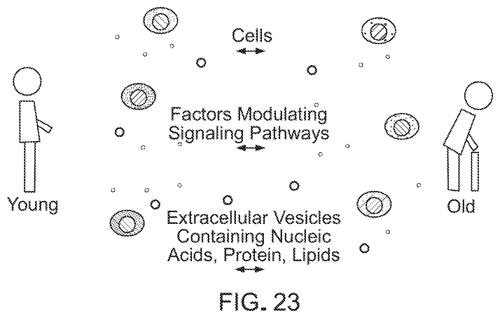

Candidate circulating mediators that may transfer aging properties include soluble factors, cells, and cellular components (FIG. 23). In addition to soluble signaling factors affecting Wnt and TGF-β signaling cascades, a wide range of additional components, including matrix metalloproteases, cytokines, growth factors, and extracellular vesicles may to be important constituents of secretory profiles. For example, Wnt regulators (such as complement C1q), modulators of TGF-β signaling (such as GDF11, activin A, and myostatin), and other signaling pathways (such as the Notch pathway and the C/EBPa pathway) are evaluated as mediators of aging, rejuvenation, and/or regeneration. For example, pro-inflammatory cytokines (such as IL-6 and IL-8), plasma chemokines for neutrophils (such as CCL11), and components of the MHC class I complex (such as 13-2 microglobulin (B2M)) are evaluated as mediators of aging, rejuvenation, and/or regeneration.

### Cell-Mediated Contributions

Candidate cellular mediators that may transfer aging properties include stem cells such as hematopoietic stem cells (HSCs). Cellular mediators can mediating rejuvenating and aging phenotypes by direct cell-cell signaling mechanisms, stem cell differentiation, and/or stem cell contribution to recipient tissues. For example, the ability of cellular mediators to integrate into adult tissues is determined. For example, donor cells (e.g., senescent donor cells) are evaluated as mediators of aging, rejuvenation, and/or regeneration.

### Additional Mechanisms

In addition to factors and cells, further means of communication may occur through extracellular vesicles (EVs), cellular byproducts containing nucleic acids, proteins, and lipid components. For example, EVs, the contents of EVs, galectin-3, and miRNAs are evaluated as mediators of aging, rejuvenation, and/or regeneration.

### Clinical Implications

The ability of youthful and senescent cells to regulate the aging of surrounding cells and tissues has potential significance for solid organ and bone marrow transplantation, particularly in clinical scenarios in which donors and recipients are not necessarily of similar age. In age-mismatched transplants, both the allograft and the recipient environment have the capacity to influence each other, suggesting a possible two-way communication with implications not only for graft survival and overall recipient outcome, but potentially also for systemic aging or rejuvenation of the recipient.

### Effects of Donor Age

Few studies have examined the association of donor age with aging-associated phenotypes, aside from those related to graft function and overall survival. In hematopoietic stem cell transplantation, older HSCs have compromised replicative capacity, with accelerated telomere shortening, increased ROS, and DNA damage accumulation, likely exacerbated by the replicative stress incurred by the transplantation process (see, e.g., Awaya et al., Biol Blood Marrow Transplant, 8:597-600 (2002); Yahata et al., Blood, 118:2941-50 (2011); and Cupit-Link et al., ESMO Open, 2:e000250 (2017)).

Transplanting senescent (vs. non-senescent) adipose mesenchymal cells into the peritoneal cavity of young mice, such that only 1 out of 10,000 cells in the recipient animal is a transplanted senescent cell, is sufficient to cause muscle weakness and other frailty-like disabilities that persist for at least 6 months (see, e.g., Example 1). Furthermore, middle-aged mice transplanted with senescent cells have a decreased lifespan compared to age-matched mice transplanted with non-senescent cells, with comparable causes of death in both groups, suggesting an acceleration of age-related pathologies rather than the promotion of any single disease. While labelled transplanted senescent cells remained within the abdominal cavity, senescent cells arising from the recipients' own cells developed in distant sites. Those observations demonstrate that spread of senescence can occur from localized populations of transplanted senescent cells, with implications for transplanted organs that carry senescent cells with them.

### Effects of Recipient Age

The effect of recipient age on graft function following transplantation has been noted in multiple cell and tissue types, with youthful or aged environments determining the regeneration capacity of transplanted tissue in animal models (see, e.g., Carlson et al., Am J Physiol, 256:C1262-6 (1989); and Young et al., Exp Gerontol, 6:49-56 (1971)). The regenerative capacity of skeletal muscle grafts in mouse models was found to correspond to the age of recipients rather than the age of the donor. Muscle grafts derived from old donors regained the capacity to regenerate when transplanted into young recipients; however, when grafts from young donors were transplanted into aged recipients, regenerative capacity appeared compromised (see, e.g., Carlson et al., Am J Physiol, 256:C1262-6 (1989)). Regenerative properties of mammary gland tissue transplants were found to depend on recipient age, as both young and old donor muscle grafts exhibited similar growth rates when transplanted into young recipients with minimal growth when placed into old recipients, though contributions of hormonal differences and replicative senescence to these results could not be excluded in this study (Young et al., Exp Gerontol, 6:49-56 (1971)).

Older recipient age also appears to increase disease recurrence subsequent to HSC transplantation in leukemia models, likely as a consequence of altered signaling in the HSC niche that occurs with aging (Vas et al., PLoS One, 7:e42080 (2012); and Vas et al., PLoS One, 7:e31523 (2012)). In other models, it has been noted that face allografts exhibit structural changes that resemble accelerated aging, although these changes may very well be related to transplantation and immunosuppression (see, e.g., Kueckelhaus et al., Am J Transplant, 16:968-78 (2016)). Despite the frequency of solid organ transplantation between donors and recipients of differing age, there has been little research on the effects of recipient age on donor organ function. The accelerated senescence observed in adult organs transferred to pediatric recipients may also reflect consequences of immunosuppression and replicative stress (see, e.g., Hodgson et al., HPB (Oxford), 17:222-5 (2015)).

### Use in Therapies

The potential of young or old cells in promoting rejuvenation or inhibiting senescence indicates that there may be interventional approaches that recapitulate these age-associated influences. An improved understanding of the molecular pathways that regulate aging and rejuvenating phenotypes may lead to other therapeutic opportunities. This aspect of aging research remains in its infancy and the utility of rejuvenating strategies needs to be determined.

### Applications in Transplantation

The recognition that donor and recipient age affect organ transplantation outcomes has led to the initiation of programs attempting to allocate organs in a manner that matches donor age and recipient age. This rationale is predominantly based on superior function of younger compared to older organs. In addition to the effects on alloimmune responses, age-matched transplants may also reduce some of the potential adverse consequences of introducing senescent cells in old donor organs into young recipients.

Additional benefits may be obtained from treating donors, recipients, or allografts with therapies that promote rejuvenation or target senescent cells. Transplanting senescent cells into young mice may shorten survival while inducing age-related phenotypes and pathologies (see, e.g., Xu et al., J Gerontol A Biol Sci Med Sci, 72:780-785 (2017); and Example 1). Therefore, organ or cell transplantation from old donors harboring senescent cells may induce aging-like dysfunction in younger recipients. Administrating senolytics at the time of transplanting senescent mesenchymal cells into the abdominal cavity of mice prevented frailty and improved survival. Even after frailty develops, senolytics are still effective, suggesting that it is possible to overcome some of the problems that may accelerate aging-related processes by transplanting organs from old donors.

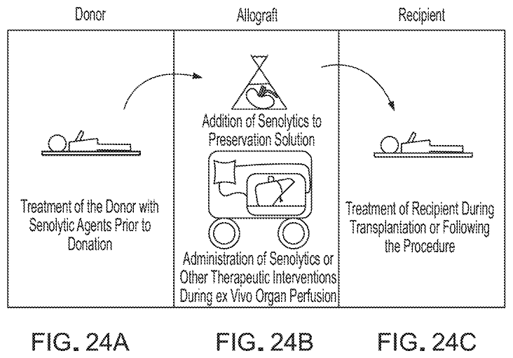

For solid organ transplantation, several windows of therapeutic targeting exist, including treatment of the donor, treatment of the recipient, or treatment of the graft itself. (FIG. 24). Treatment of the donor with senolytic agents prior to donation, for example, is one means of depleting senescent cells within the allograft. This treatment has relevance in the setting of living donor transplantation, as deceased donor transplantation situations do not permit for the advanced planning required for administration of such compounds. Adverse effects of certain senolytic agents at this time, such as cytopenias caused by Navitoclax or, possibly, delayed wound healing, limit application, as minimum harm to the donor is required. Transplant recipients may also receive senolytic or senomorphic agents following transplantation. Treatment of the allograft itself, following organ procurement and prior to transplantation into the recipient, may provide another opportunity for intervention. Although current protocols focus on minimizing the duration of cold ischemia time to reduce the adverse effects of ischemia on allograft function, brief treatments with senolytic agents, e.g., by adding them to organ preservation solutions, provide a unique opportunity for targeted delivery of these compounds. In addition, emerging preservation concepts allowing for prolonged ischemic times permit the administration of such agents. Indeed, the emerging technology of ex vivo organ perfusion, currently under development as a means of increasing organ usage in transplantation, may provide additional flexibility. Such systems also allow assessment of organ function prior to transplantation. For example, a brief exposure of freshly-procured clinical adipose tissue with senolytics was sufficient to reduce senescent cell abundance by apoptosis, while leaving non-senescent cells intact (see, e.g., Example 1). In addition to small molecules, nucleic acid and viral vector-based therapeutics, immunization, as well as cellular therapies may be employed, potentially allowing for more specific targeting of senescent cells.

### Conclusions

The ability of youthful and senescent cells to exert systemic rejuvenating or aging influences has significant clinical implications. The identification of circulating factors and cells as potential mechanisms by which these cells exert their effects has been instrumental in developing potential therapeutic interventions that promote rejuvenation while minimizing effects of aging. Identification of additional mediators promoting rejuvenation provide new opportunities for intervention. As individuals of older age are increasingly becoming transplant donors and recipients, these insights should help clarify the appropriate applications for therapeutics that promote rejuvenation and target senescent cells.

### Example 4: Senolytic Agents Prevent mt-DNA-Induced Inflammation and Promote Organ Transplant Survival

This example demonstrates that cf-mt-DNA accumulates in aging promoting an augmented immunogenicity through the activation of dendritic cells (DCs) that initiate Th1 and Th17 CD4⁺ T cell responses in transplant recipients. Importantly, ischemia/reperfusion injury (IRI), an unavoidable element of transplantation, resulted in a marked increase of systemic cf-mt-DNA and age-associated inflammatory responses in old, but not young, animals. Blocking TLR9, the ligand of mtDNA, dampened the activation of old DC, ameliorated associated pro-inflammatory T cell responses, and extended old allograft survival. Of translational relevance, comparable events were also observed clinically, with cf-mt-DNA levels being significantly elevated in older deceased organ donors. Moreover, isolated mt-DNA from deceased organ donors specifically activated human DCs. In experimental models, senolytic agents cleared senescent cells, decreased systemic cf-mt-DNA levels, and attenuated age-associated inflammatory profiles. Treating donor animals promoted the survival of older murine allotransplants. Thus, the accumulation of cf-mt-DNA was identified as a key player in inflamm-aging, DC activation and augmented age-associated immune response in experimental and clinical models.

### Results

DCs from Old Mice are Abundant in Lymph Nodes, Exhibit Increased Markers of Activation, and Drive Naïve CD4⁺ T Cells Towards Th1 and Th17Pro-Inflammatory States

The impact of aging on DC activation and CD4⁺ T cell fate was previously characterized, and it was shown that aging activates DCs specifically with subsequent Th17 driven alloimmune responses (Oberhuber et al., Circulation 132:122-131 (2015)).

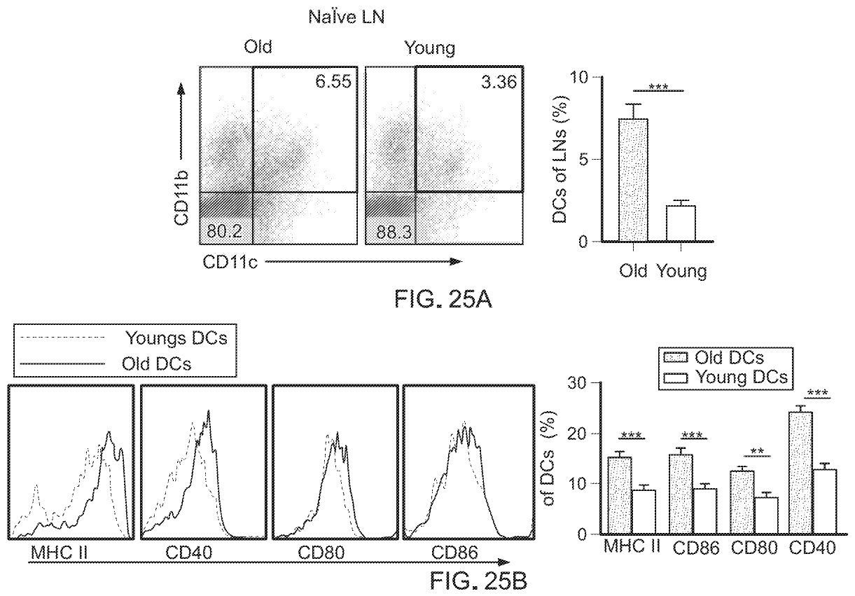

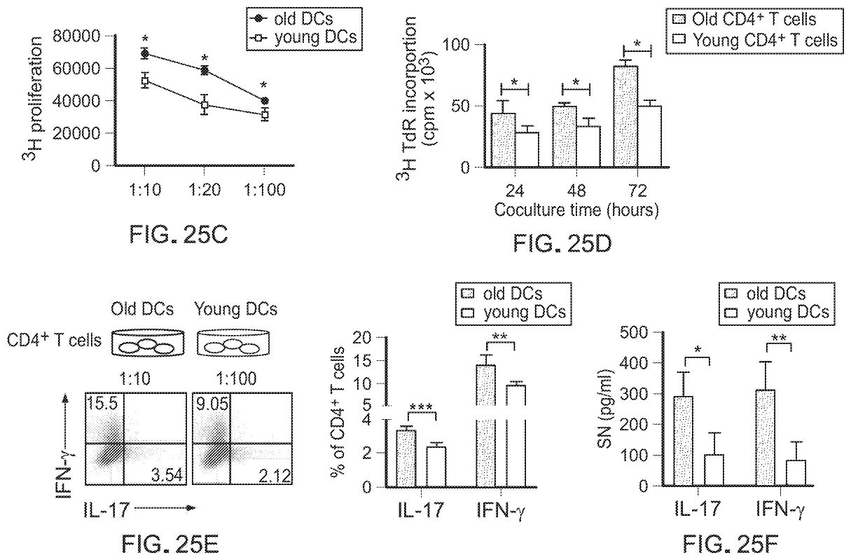

Here, increased frequencies of peripheral lymphoid CD11b⁺CD11c⁺DCs were found in old mice (FIG. 25A). Next, the impact of old DCs on T cell responses was analyzed, and the phenotypic maturity of freshly-isolated CD11b⁺CD11c⁺DCs from young and old mice was delineated. Results showed an elevated expression of MHC class II in addition to an enhanced expression of the costimulatory molecules, CD40, CD80, and CD86 in old, but not young, DCs (FIG. 25B).

Notably, flow-sorted, freshly isolated old C57BL/6 mouse CD11b⁺CD11c⁺DCs induced ˜2-fold higher proliferation of naive allogeneic (DBA/2J) splenic CD4⁺ T cells when compared to DCs from young mice. Of additional relevance, numbers and proliferative responses of CD4⁺ T cells to CD11c⁺CD11b⁺DCs had increased at varying DC-CD4⁺ T cell ratios (FIG. 25C). Moreover, T cell proliferation after 72 hours of co-culture was significantly greater after stimulation with old compared to young mouse DCs (FIG. 25C).

To determine whether age-specific differences reflected different kinetic responses, MLR cultures were performed by 24, 48, and 72 hours. At each time point, CD4⁺ T cell proliferation had significantly increased in response to stimulation with old DCs (FIG. 25D).

Taken together, these results underscore an age-specific proliferative capacity (FIG. 25C) and kinetic responses (FIG. 25D) of naive allogeneic CD4⁺ T cells to splenic CD11b⁺CD11c′ DC subsets in mixed lymphocyte reactions (MLR).

With CD11b⁺CD11c⁺DCs recognized as instigators of Th1 and Th17-driven inflammatory responses (Nowak et al., J. Exp. Med. 206:1653-1660 (2009)), whether old DCs co-cultured with DBA/2J CD4⁺ T cells had a direct effect on Th1 and Th17 cell differentiation was next examined. Indeed, at varying DC:CD4⁺ T cell ratios, DBA/2J CD4⁺ T cells cultured with old mouse DCs showed significantly higher levels (2-fold increase) of IFN-γ and IL-17 production (FIG. 25E), suggesting an age-specific Th1 and Th17 cell differentiation promoted by old DCs. Consistent with these findings, supernatants of T cells co-cultured with old DCs showed an enhanced production of IFN-γ and IL-17, as quantified by ELISA (FIG. 25F).

Collectively, these results show an age-specific activation of DCs that promoted Th1 and Th17 responses, two prominent CD4⁺ T cell pro-inflammatory subsets that are involved in IRI and transplant rejection.

### Adoptive Transfer of Old DCs Reduces Allograft Survival

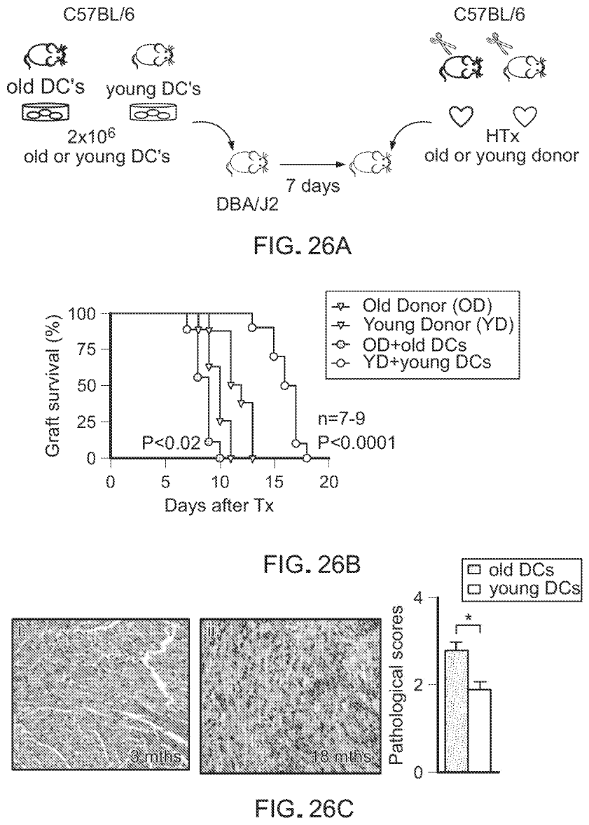

Increasing evidence suggests that DCs impact allograft survival age-specifically. Young donor-type CD11b⁺CD11c⁺DCs generated in vitro can prolong allograft survival (Min et al., J. Immunol. 164:161-167 (2000)). Moreover, depletion of DCs from old organs prolonged graft survival (Oberhuber et al., Circulation 132:122-131 (2015)). To test whether old DCs affect transplant survival in vivo, 2×10⁶ (>95% purity) DCs were isolated from young and old C57BL/6 mice and adoptively transferred into DBA/2J mice i.v. 7 days prior to the engraftment of vascularized heterotopic C57BL/6 cardiac transplants (FIG. 26A). As shown in (FIG. 26B), transfer of old CD11b⁺CD11c⁺DCs significantly reduced median graft survival from 11 days in untreated controls to 8 days (p<0.02). Conversely, adoptive transfer of young CD11b⁺CD11c⁺DCs prolonged graft survival from 13 to 18 days (p<0.0001).

Histological examination of cardiac allografts revealed a dramatic difference between recipients that had received either adoptively transferred young or old DCs. In line with the augmented activation of old CD11b⁺CD11c⁺DCs in vitro and the accelerated graft rejection, it was observed that cardiac allografts of mice that had received adoptively transferred old DCs showed multiple inflammatory foci. In contrast, only few inflammatory cells were detected in recipients of young DCs. Accordingly, pathological scores of animals that had received either old or young DCs were significantly different (FIG. 26C; p<0.05).

Collectively, these data show that DCs are activated and promote CD4⁺ T cell infiltration in an age-specific manner, resulting in accelerated allograft rejection.

DC Activation Results from an Age-Specific Increase of Circulating Cf-Mt-DNA

Aging is associated with low grade inflammation, contributing to the development of several diseases including atherosclerosis, Alzheimer's, and malignancies (Sanada et al., Frontiers in cardiovascular medicine 5:12 (2018)). Conceptually, inflamm-aging has been linked to continuous stimulation of professional antigen presenting cells (APCs) including dendritic cells, a process termed “Garb-aging” in which clearance of damage-associated molecular patterns (DAMPs) is impaired (Franceschi et al., Trends Endocrinol. Metab. 28:199-212 (2017)). These processes become even more relevant when responding to injuries such as IRI. Indeed, both aging and IRI are associated with increased DAMP release. Therefore, whether DAMPs, specifically cf-mt-DNA levels, increase with aging was investigated.

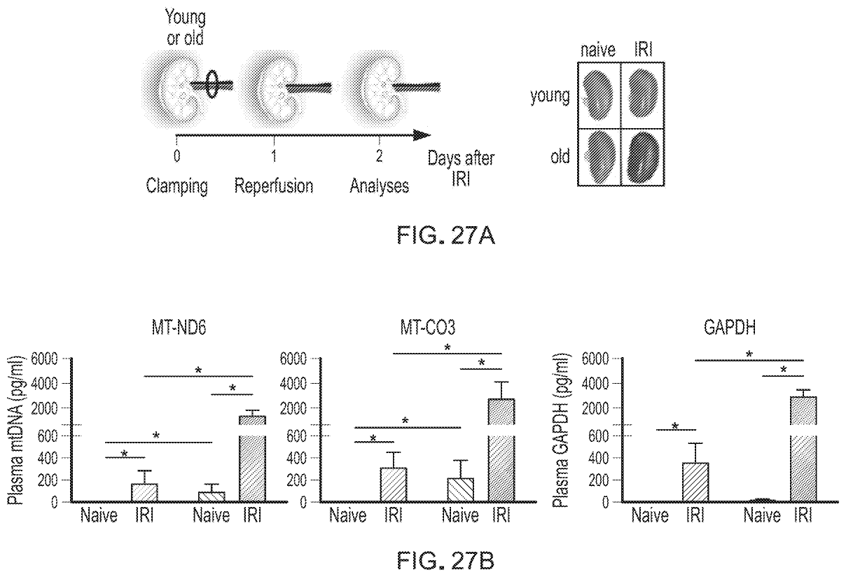

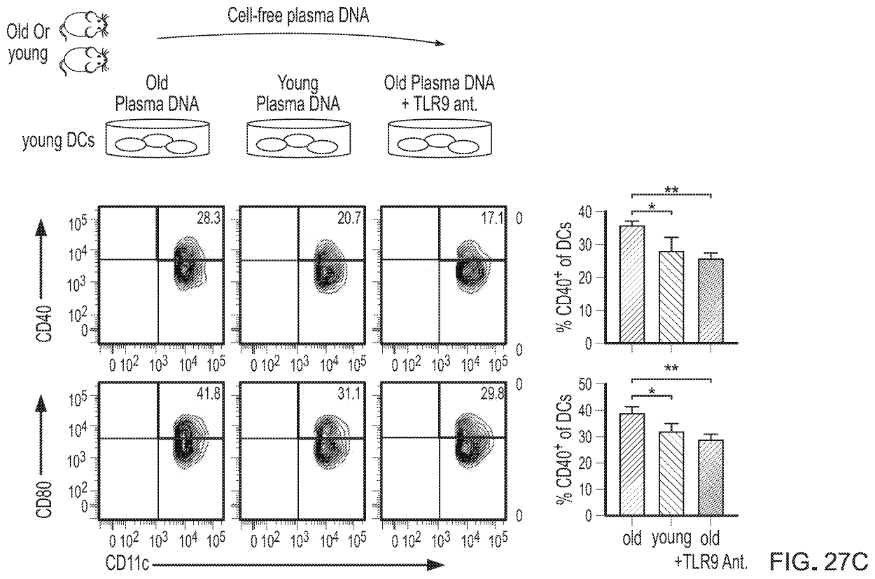

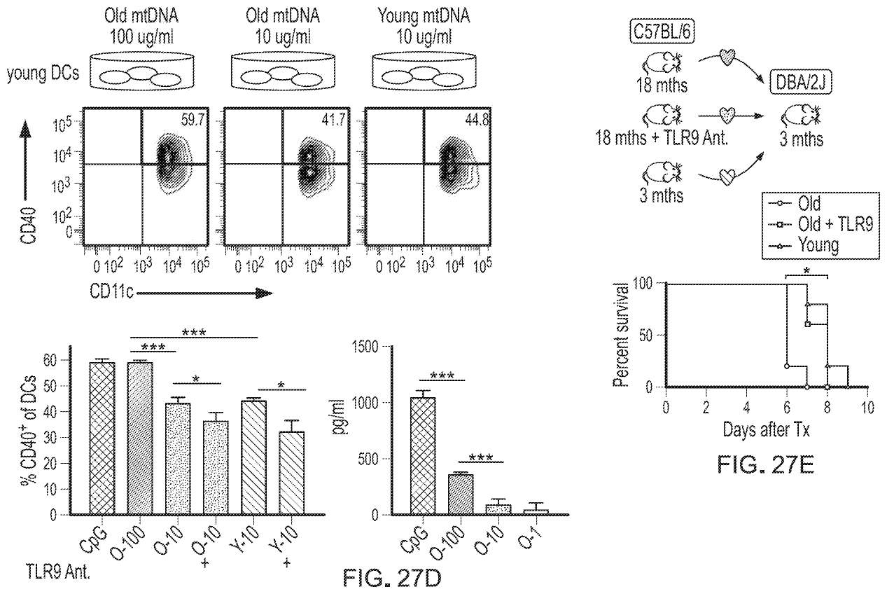

Cf-mt-DNA levels were quantified with primers for the mitochondrially encoded NADH dehydrogenase subunit 6 (MT-ND6) and cytochrome c oxidase subunit III (MT-CO3) of the electron transport chain. Old mice exhibited increased levels of cf-mt-DNA, while levels were not detectable in young animals (FIG. 27B). These differences became significantly more pronounced after IRI. Notably, old mice that underwent renal IRI (FIG. 27A) had a 15-fold increase in cf-mt-DNA levels compared to young animals (FIG. 27B). Systemic GAPDH was absent from serum of old and young naïve mice but became detectable in mice subjected to IRI (FIG. 27B).

Cf-mt-DNA has the capacity to induce sterile inflammation as a response to injury through a TLR9 dependent pathway. To test whether augmented systemic mt-DNA levels in old mice activated DC, young and old plasma DNA was cultured with young DC isolated from naïve C57BL/6 mice (3 months). Old plasma-DNA activated co-stimulatory molecules (CD40 and CD80) in an age-specific fashion. Remarkably, after adding ODN 2088, a TLR9 antagonist, the upregulation of co-stimulatory molecules had been attenuated in the presence of old cell-free plasma DNA (FIG. 27C).

Next, these effects were tested in more detail using isolated mt-DNA at different concentrations, and a dose-dependent upregulation of CD40 was consistently detected. This effect was attenuated when the receptor for cf-mt-DNA was blocked by a TLR9 antagonist (FIG. 27D).

Of note, comparable mt-DNA concentrations (10 μg) from either old or young plasma did not impact DC activation, suggesting that cf-mt-DNA quantities rather than qualitative differences in old animals determined DC activation (FIG. 27D). It also was observed that levels of IL-6, a pro-inflammatory cytokine that is part of the SASP, displayed a dose-dependent increase in the culture supernatant upon mt-DNA stimulation (FIG. 27D). Moreover, in vivo TLR9 blockade resulted in prolonged allograft survival (FIG. 27E).

Taken together, these findings indicate that increasing amounts of cf-mt-DNA activate DC in an age-specific fashion via TLR9. Old DC promoted age-specific alloimmune responses that accelerated the rejection of older organs.

Senescent Cells Accumulate with Aging in Clinical and Experimental Models and are a Key Source of cf-mt-DNA

Mutations of mitochondrial DNA increase with aging. Moreover, dysfunctional mitochondria accumulate and contribute to cellular senescence in vitro and in vivo. In addition, accumulation of mitochondrial reactive oxygen species has been linked to cellular senescence, with the mitochondrial dysfunction-associated senescence (miDAS) phenotype being involved. Thus, to investigate senescent cells as a potential source of increased cf-mt-DNA levels in old animals, IHC stains were performed for the cyclin-dependent kinase inhibitors p21^{cip1} and p16^{Ink4a}, both markers of cellular senescence. Additionally, murine kidneys were tested for lysosomal-origin-β-galactosidase, an enzyme activity that is increased in many senescent cells.

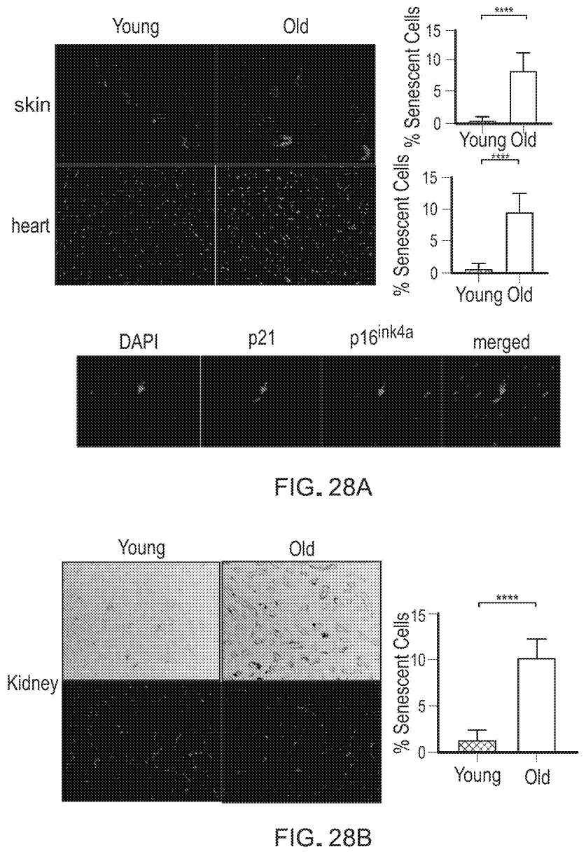

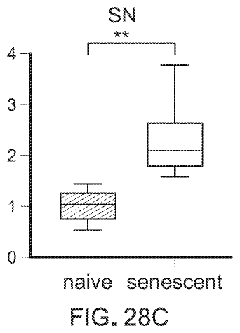

Skin and hearts of older mice contained significantly elevated numbers of senescent cells defined by p16^{Ink4a}/p21^{cip1} double positivity (FIG. 28A). Similarly, kidneys from older donors showed higher numbers of sa-β-gal positive cells (FIG. 28B).

To link the accumulation of senescent cells to augmented cf-mt-DNA in aging, preadipocytes were isolated from C57BL/6 mice and irradiated with 10 Gy to induce senescence. Significantly higher yields of cf-mt-DNA were observed in the supernatant of senescent adipocytes (FIG. 28C), suggesting senescent cells as a key source of the increased cf-mt-DNA in aging.

### Old Human Organ Donors Show Increased Levels of Systemic Cf-Mt-DNA Capable of Activating DCs

Experimentally, it was shown that old mice have increased systemic levels of cf-mt-DNA resulting in DC activation and an age-specific pro-inflammatory response. It was also shown that senescent cells are an explicit source of IRI-induced cf-mt-DNA release causing old DCs to induce Th1 and Th17 T cell-driven alloimmune responses when transplanting organs from older donors.

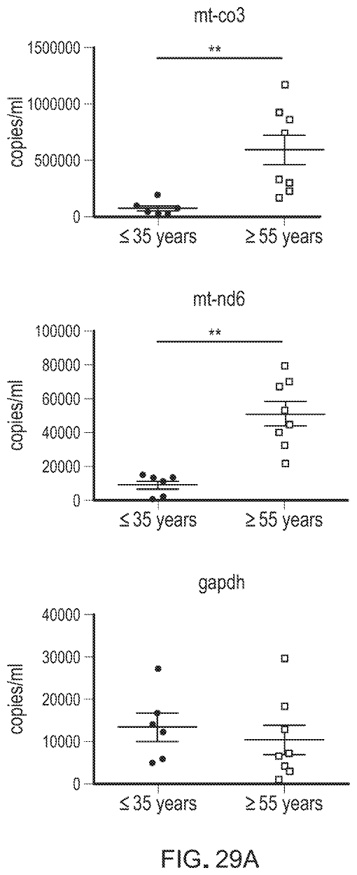

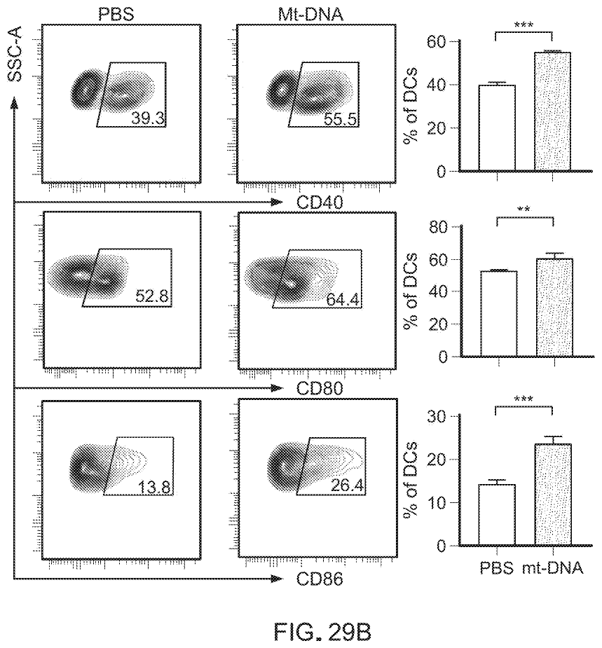

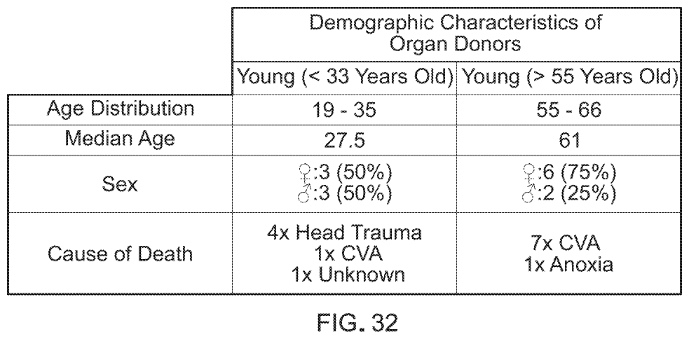

To test the clinical relevance of these findings, cf-mt-DNA in plasma samples of older (>55 years) and younger (<35 years) human organ donors were analyzed (Clinical characteristics detailed in FIG. 32). Notably, young organ donors had consistently lower plasma levels of cf-mt-DNA compared to old donors (FIG. 29A), findings that were consistent with the experimental data.

Next, the capacity of human mt-DNA to induce DC activation was delineated. Indeed, when stimulating DCs with human mt-DNA, an upregulation of the costimulatory molecules CD40, CD80, and CD86 (FIG. 29B) that are involved in mounting alloimmune responses was observed.

### Senolytics Decrease Cf-Mt-DNA Levels and Prolong Survival of Old Murine Cardiac Transplants

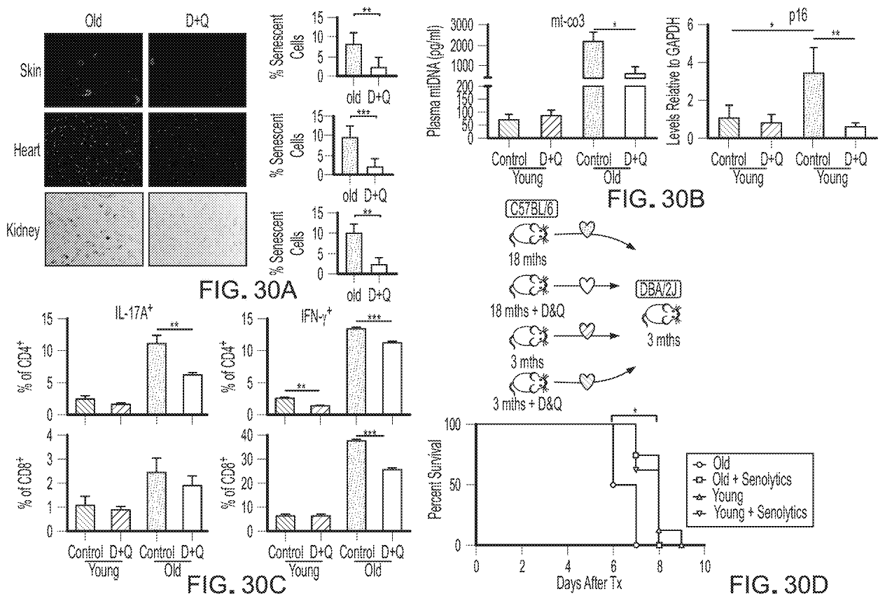

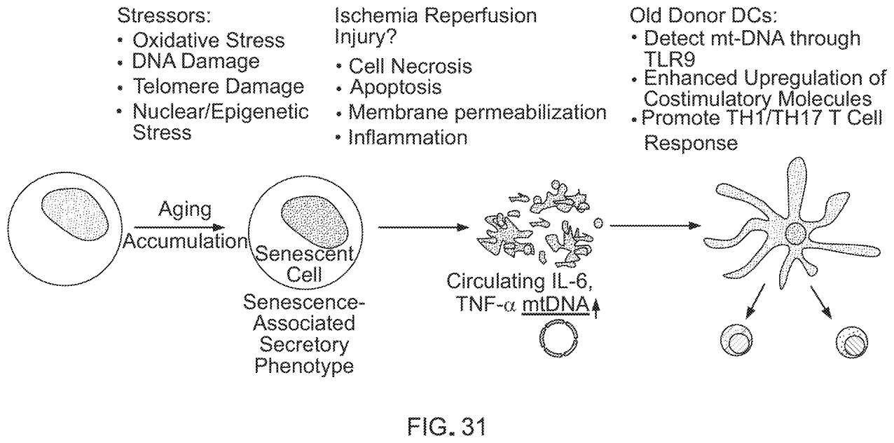

To test, in vivo, the consequences of augmented cf-mt-DNA levels in aging, animals were treated with senolytics (Dasatinib plus Quercetin) that induce selective apoptosis of senescent cells. Treating old C57BL/6 mice (18 months) with Dasatinib (D, 5 mg/kg) plus Quercetin (Q, 50 mg/kg) significantly decreased p16^{Ink4a}/p21^{Cip1} cells in skin and hearts. Moreover, sa-β-gal positive cells had significantly decreased in kidneys subsequent to D & Q treatment (FIG. 30A).

Next, the effects of senolytics in relevant pre-clinical disease models were tested. Senolytic treatment prior to renal IRI substantially reduced local and systemic levels of cf-mt-DNA in old mice. Moreover, the senescent cell marker p16^{Ink4a+} was significantly reduced in old kidneys after the treatment with senolytics (FIG. 30B). In addition, senolytics also reduced systemic levels of pro-inflammatory T cells including CD8⁺IFN-γ⁺, CD4⁺IFN-γ⁺, and CD4⁺IL-17⁺ in old animals after IRI (FIG. 30C).

Next, to examine if the clearance of senescent cells prolonged graft survival, old and young donor mice (n=8) were treated with a single dose of D+Q prior to organ procurement. Indeed, senolytics specifically prolonged the survival of cardiac allografts from older donors (FIG. 30D).

Collectively, these results demonstrate a link between senescent cell clearance and reduced cf-mt-DNA systemic levels in relevant experimental and clinical models.

Importantly, clearance of senescent cells in old animals decreased cf-mt-DNA levels that ameliorated DC activation and pro-inflammatory T cell responses. Of clinical relevance, senolytics improved the consequences of IRI and prolonged allograft survival.

### Materials and Methods

### Animals

Young (2 months) C57BL/6 (B6) and DBA/2J mice were purchased from Charles River Laboratory, Wilmington, Mass.; old (18 months) C57BL/6 mice were from the National Institute of Aging (NIA, Bethesda, Md.), which uses the same colony of young C57BL/6 mice as Charles River Laboratory. Mice were anesthetized with ketamine, and in case of sacrifice, blood was collected by heart puncture in heparin-coated tubes.

### Deceased Organ Donors:

Deceased donors were part of a prospective trial as described elsewhere and enrolled in the normothermic arm (Niemann et al., N. Engl. J. Med. 373:2687 (2015)). Samples were convenience samples and collected during donor management, processed onsite, and subsequently stored at −80 degrees for further analysis.

### Assay for Immune Activity in Recipient Draining Lymph Nodes (DLN) and Transplanted Hearts

Heart grafts were perfused in situ under gaseous anesthesia (Metofane; Schering-Plough) with 10 mL of HBSS and then 5 mL of collagenase (1 mg/mL). Hearts were then removed, minced into small pieces, and digested in collagenase containing DNase (100 μg/ml) at 37° C. for 60 minutes. Cells were filtered through a 70-μm nylon cell strainer (BD Biosciences, Franklin Lakes, N.J.). Graft-infiltrating cells (GICs) were isolated by density centrifugation using Lympholyte-M (Cedarlane Laboratories, Hornby, Ontario, Canada) for 30 minutes at 800×g, and were washed twice with complete medium. The presence of cells within GICs or bulk recipient DLNs was assayed by the addition of 5×10⁴ gamma-irradiated (20 Gy) cells at the start of 72 hours one-way MLR (old or young B6→DBA/2J). Naive bulk C57BL/6 spleen cells were used as a source of stimulators (2×10⁶/mL), and nylon-wool enriched DBA/2J splenocytes were used as responder T cells (2×10⁶/mL).

Adoptive Transfer of CD11b⁺CD11c⁺ Subsets

Sorted old and young CD11b⁺CD11c⁺DCs were washed extensively in HB SS and injected (2×10⁶ in 400˜500 μl of HB SS) into DBA/2J mice via the lateral tail vein. After 7 days, mice received vascularized heterotopic B6 heart transplants, as described below. For ex vivo functional studies, spleens were removed either 7 or 12 days after adoptive transfer of DCs.

### DC Isolation and Sorting

DCs were isolated from the spleens of naïve old and young mice. Spleens were disaggregated and digested for 15 minutes with 10 mL of type IV collagenase (200 μg/mL; Sigma-Aldrich, St. Louis, Mo.) in HBSS supplemented with 100 μg/mL DNase (Roche, Mannheim, Germany). After digestion, splenocytes were collected by centrifugation at 500×g, and erythrocytes were lysed by hypotonic shock using 0.15 M NH₄Cl. DCs were isolated immediately after splenocyte preparation. DCs were enriched from fresh splenocytes by metrizamide (16.5 or 14.5% (w/v), respectively) density centrifugation at 500×g for 15 minutes at room temperature (20° C.). For purification by sorting, the buffy layer was labeled with anti-CD11c, anti-CD11b, and anti-CD8α for 30 minutes at 4° C. Cells were washed, incubated for 5 minutes at 4° C. with cation-free HBSS containing 1% (v/v) FCS and 10 mM EDTA to disaggregate cell clusters, and then resuspended in complete medium. CD11b⁺ CD11c⁺DC populations, with high forward- and side-scatter profiles, were sorted using a Coulter EPICS Elite (Beckman Coulter, Hialeah, Fla.) to >95% purity. For isolation of CD11c⁺DCs, single cell suspensions were obtained from hearts of young (8-12 weeks) and old (18 months) C57BL/6 WT mice. Briefly, hearts were procured and washed 3× with Ca2⁺- and Mg2⁺-free PBS. Tissue was then cut into 5 mm pieces and placed in tissue extraction buffer (5 mM EDTA, 2 mM 2-ME in PBS), and incubated with continuous, brisk stirring at 37° C. for 30 minutes. The suspension was filtrated through a 70 μm filter. CD11c⁺ DCs were then isolated using EasySep™ Mouse CD11c Positive Selection Kit (Stemcell Technologies) according to the manufacturer's protocol.

### PBMC Isolation and Differentiation of Human DCs

PBMCs were isolated via density gradient centrifugation using SepMate (Stemcell) tubes and lymphoprep (Stemcell) density gradient medium. Briefly, blood was obtained from healthy donors, diluted 1:1 with PBS, and added to the SepMate Tubes containing lymphoprep density gradient medium. Tubes were then centrifuged at 1200 g for 10 minutes, and the top layer containing enriched PBMCs was poured off into a new tube. Subsequently PBMCs were washed twice with PBS. 1-1.5×10\hat{ }6 PBMC were then plated in a 25 cm² cell culture flask (Corning Incorporated) in RPMI 1640 (gibco) supplemented with 10% fetal calf serum, 100 μg/mL streptomycin, 100 U/mL penicillin, 2 mM glutamine, and 1 mM sodium pyruvate for 2.5 hours at 37° C./5% CO₂. Subsequently, the culture medium containing non-adherent cells and attached cells were washed three times with PBS. Monocytes were then harvested after short centrifugation, and 5×10″5 cells/mL were re-cultured in 25 mL of described culture medium containing additional GM-CSF (50 ng/mL) and IL-4 (30 ng/mL) (both from Promega) for 6 days. Medium was replaced after 3 days with fresh supplemented medium re-plating all non-adherent cells by centrifuging supernatant. DCs were then plated in a 48 well plate at a density of 1×10\hat{ }6 cells/mL, cultured overnight, and subsequently stimulated with 10 μg/mL isolated mt-DNA/PBS.

### Mixed Lymphocyte Reaction

Bulk splenocytes or T cells from naive or DC-primed DBA/2J mice were enriched by a single passage through nylon wool columns (45 minutes at 37° C.) and used as responders. A total of 2×10⁵ cells were placed in each well of 96-well round-bottom plates, and varying numbers of gamma-irradiated (20 Gy), sorted young or old CD11b⁺CD11c⁺DCs (DBA/2J, B6) were added as stimulators. In some experiments, human rIL-2 (50 U/mL; Genetics Institute, Cambridge, Mass.) was added at the start of culture to test for reversal of hypo-responsiveness. Cultures were incubated in complete medium for 72 hours unless otherwise specified in a humidified atmosphere of 5% CO₂ in air. [³H]TdR (1 μCi in 10 μL) was added to each well for the final 18 hours of culture. Cells were harvested using a multiple-well harvester, and [³H]TdR incorporation was determined in a liquid scintillation counter. Results are expressed as the mean counts per minute±1 SD from triplicate cultures.

### Detection of Intracellular Cytokines

Cytokines were detected intracellularly in responder DBA/2J T cells after 72 hours MLR using normal bulk C57BL/6 splenocytes as stimulators (stimulator:responder ratio, 1:1) or CD11b⁺CD11c⁺DC cells. T cells were then re-stimulated with plate-bound hamster anti-mouse CD3 (10 μg/mL) and soluble hamster anti-mouse CD28 (10 μg/mL) for 5 hours at 37° C. in the presence of brefeldin A (10 μg/mL; Sigma-Aldrich). Thereafter, cells were washed with 1% (v/v) FCS/PBS, fixed with 4% (w/v) paraformaldehyde (20 minutes, 4° C.), and permeabilized with 0.15% (w/v) saponin/1% (v/v) FCS/PBS for 15 minutes at 4° C. Cells were then labeled and incubated for 30 minutes at 4° C. with 1) anti-CDR, 2) anti-CD4, and 3) anti-CD8α. Intracellular cytokines were detected by the addition of conjugated anti-IFN-γ and anti-IL-17 mAbs, all purchased from BD PharMingen. After staining, cells were washed with 1% (v/v) FCS/PBS, fixed with 1% (w/v) paraformaldehyde, and analyzed immediately using a BD flow cytometer. Cells stained with appropriate isotype-matched Ig (BD PharMingen) were used as negative controls.

### ELISA

Splenocytes prepared from DBA/2J mice 5 days after heart transplantation were re-stimulated with bulk donor-type (C57BL/6) splenocytes as described for MLR. Supernatants were harvested after 72 hours of co-culture. To assess cytokine production over a discrete period (24 hours) at the peak of T cell proliferation, cells were harvested after a 72 hours co-culture, washed, and resuspended in fresh complete medium for additional 24-hour stimulation with anti-CD3 and anti-CD28 mAbs. ELISA for mouse IFN-γ and IL-17 in culture supernatants was performed using reagents purchased from BD PharMingen and following the manufacturer's recommended procedures.

### Heterotopic Heart Transplantation

Vascularized heart grafts from C57BL/6 donor mice were heterotopically transplanted into old or young DBA/2J recipient mice using a modified cuff technique for revascularization as described elsewhere (Niemann et al., N. Engl. J. Med. 373:2687 (2015)). Graft function was monitored by daily palpation, and graft rejection was defined as total cessation of contractions and verified by macroscopic inspection and histology.

### Immunohistochemistry

Mice were anesthetized with isoflurane and shaved, and the skin over the back was removed with a scalpel. To procure kidneys and hearts, a midline incision was performed, and the rib cage was divided laterally to the thoracic arteries. Kidneys and heart were harvested respectively, washed with PBS, and embedded in 10% formalin.

For assaying sa-β-gal activity, kidneys were transferred into 30% sucrose after 6 hours and incubated overnight at 4° C. Afterwards, samples were frozen in O.C.T, cut into 5-um sections using a cryostat, and added onto Superfrost Plus Microscope Slides. Activity was assayed performed using a sa-βa-gal kit (Cell Signaling) according to the manufacturer's protocol. Slides were covered using Vectashield mounting medium with DAPI and analyzed using a bright field microscope.

For immunofluorescence, heart and skin were kept in formalin for 18 hours and subsequently embedded in paraffin. The paraffin sections were deparaffinized and rehydrated, followed by antigen retrieval using sodium citrate buffer (pH 6). After 3 washes with TBS, sections were incubated with 5% normal donkey serum (Jackson ImmunoResearch Lab Inc, West Grove Pa.) for an hour at room temperature. Slides were then incubated with mouse anti-p16^{Ink4a} (1:500, Abcam, ab54210) and rabbit anti-p21^{cip1} (1:200, Abcam, ab188224) primary antibodies overnight at 4° C. Slides were washed 3 times and incubated with Cy3 conjugated Donkey anti-rabbit secondary antibody (Jackson ImmunoResearch Lab, 1:300) and Alexa Fluor 647 conjugated Donkey anti-mouse secondary antibody (Invitrogen, 1:300). Samples were counterstained with Hoechst and then washed 3 times with TBS, and the slides were mounted with Prolong Gold anti-fade mounting media (Invitrogen).

The percentage of senescent cells was defined as the number of p16/p21 double positive (FIG. 28A) and sa-β-Gal positive (FIG. 28B) DAPI positive cells.

### Flow Cytometric Analyses

CD3, CD4, CD8α, CD11b, CD11c, CD40, CD80, CD86, H2K^{b}, and IA^{bβ}-chain mAbs were used for immunophenotyping. Draining lymph nodes (dLN) cells or splenocytes were isolated and suspended in complete RPMI1640 with 10% FCS at a density of 2.5×10⁶/mL. Mononuclear cell suspensions were re-stimulated with PMA (50 ng/mL) and ionomycin (500 ng/mL) (Sigma) and treated with Golgi Stop (1 μg/10⁶ cells) (BD PharMingen, San Jose, Calif.) for 4-24 hours. Cells were procured, washed in staining buffer containing 1% FCS, 0.1% NaN₃ in PBS, and blocked with anti-CD16/CD32 antibodies. Following another wash step, cells were stained with fluorescence labeled antibodies for 30 minutes in the dark at 4° C. Cells were then washed, fixed, and permeabilized using Fix and Perm® cell permeabilization reagents (Caltag Laboratories, Burlingame, Calif.). Subsequently, cells were stained for intracellular cytokines with conjugated rat anti-mouse IFN-γ and IL-17 antibodies.

All antibodies were purchased from BD PharMingen. Flow cytometry measurements were performed using a FACSCalibur system (BD), and data were analyzed using FlowJo (Tree Star, Ashland, Oreg., USA).

### Real-Time PCR

Mitochondria were isolated from whole liver tissue of wild type male C57BL/6 mice using Mitochondria Isolation Kit for Tissue (Thermo Fisher Scientific, Waltham, Mass., USA). Mitochondrial DNA was subsequently extracted from the isolated mitochondria using QIAmp Blood & Tissue (Qiagen, Hilden, Germany). DNA extraction from plasma was performed using QIAamp DNA Mini and Blood Mini Kit according to the manufacturer's protocol (Qiagen, Hilden, Germany). For real-time PCR reactions, mouse cytochrome c subunit III (qMmuCEP0060078), mouse NADH dehydrogenase 6 (qMmuCEP0062889), and GAPDH (qMmuCEP0039581) measurements were performed with Taqman primers and probes from Bio-Rad. A real-time PCR standard curve was created to quantify cf-mt-DNA concentration by using purified mt-DNA and cytochrome c oxidase subunit III as target. Samples that produced no PCR products after 40 cycles were considered undetectable and the Ct number set to 40 for statistical purposes.

### Statistical Analysis

Kolmogorov-Smirnov and d'Agostino & Pearson omnibus normality tests were applied to verify Gaussean distribution before using two-sided one-way-ANOVA or Student's T-test with Tukey's post-test or Dunnett's Multiple Comparison test for proofing statistical significance. Non-parametric data were analyzed using Friedmann test. For in vivo survival data, Kaplan-Meier survival graphs were constructed, and the log-rank comparisons of the groups were used to calculate p values. The level of significance was chosen to be at p<0.05 (GraphPadPrism 5.01, La Jolla, Calif., USA).

### Example 5: Treating Transplant Donors

A transplant donor (e.g., an organ donor) is treated with a compositing including one or more senotherapeutic agents (e.g., one or more senolytic agents such as D+Q) prior to donation. Treatment of the donor with senolytic agents prior to donation can, for example, depleting senescent cells within the transplanted cells, tissues, organs, and/or a population of cells not in the form of tissue or an organ.

The transplant donor can be age-matched or age-mismatched with the intended recipient. For example, the transplant donor can be an older donor (e.g., over age 55). In some cases, the transplant donor can be on life support.

### Example 6: Treating Transplant Recipients

A transplant recipient (e.g., an organ recipient) is treated with a compositing including one or more senotherapeutic agents (e.g., one or more senolytic agents such as D+Q). The compositing including one or more senotherapeutic agents can be administered prior to, during, and/or following the transplantation. Treatment of the recipient with senolytic agents can, for example, maintain the regenerative capacity of the graft in the environment of the recipient.

The transplant recipient can be age-matched or age-mismatched with the transplant donor. For example, the transplant recipient can be an older recipient (e.g., over age 55).

### Example 7: Treating Grafts to be Transplanted

Cells, tissues, organs, and/or a population of cells not in the form of tissue or an organ to be transplanted are treated with a compositing including one or more senotherapeutic agents (e.g., one or more senolytic agents such as D+Q) after harvesting, but prior to transplantation. For example, one or more senotherapeutic agents can be used in an ex vivo organ perfusion to treat cells, tissues, organs, and/or a population of cells not in the form of tissue or an organ to be transplanted. The ability to administer a compositing including one or more senotherapeutic agents to the graft to be transplanted (rather than the transplant donor and/or the transplant recipient) can limit side effects to transplant recipients.

Treatment of cells, tissues, organs, and/or a population of cells not in the form of tissue or an organ to be transplanted with senolytic agents can, for example, deplete senescent cells within the cells, tissues, organs, and/or a population of cells not in the form of tissue or an organ to be transplanted and/or maintain the regenerative capacity of the cells, tissues, organs, and/or a population of cells not in the form of tissue or an organ to be transplanted.

In some cases, a compositing including one or more senotherapeutic agents can be used together with one or more organ preservation solutions. In some cases, a compositing including one or more senotherapeutic agents can be used together with reduced temperatures.

The transplant donor and the transplant recipient can be age-matched or age-mismatched. For example, the transplant donor, the transplant recipient, or both can be older (e.g., over age 55).

### Other Embodiments

Embodiment 1. A method for providing a recipient mammal with a graft, wherein said method comprises: (a1) administering a composition comprising a senolytic agent to a donor mammal providing said graft before said graft is obtained from said donor mammal, (a2) administering said composition to said recipient mammal, or (a3) contacting said graft with said composition, and (b) providing said recipient mammal with said graft.

Embodiment 2. The method of Embodiment 1, wherein said recipient mammal is a human.

Embodiment 3. The method of any one of Embodiments 1-2, wherein said graft is a graft from a human donor.

Embodiment 4. The method of Embodiment 3, wherein said human donor is over 55 years of age.

Embodiment 5. The method of any one of Embodiments 1-4, wherein said graft is a tissue graft.

Embodiment 6. The method of Embodiment 5, wherein said tissue graft is bone marrow.

Embodiment 7. The method of any one of Embodiments 1-4, wherein said graft is an organ graft.

Embodiment 8. The method of any one of Embodiments 1-4, wherein said graft is a population of cells not in the form of tissue or an organ.

Embodiment 9. The method of Embodiment 8, wherein said population of cells comprises hematopoietic stem cells or blood.

Embodiment 10. The method of any one of Embodiments 1-9, wherein said senolytic agent is dasatinib or quercetin.

Embodiment 11. The method of any one of Embodiments 1-10, wherein said composition comprises dasatinib and quercetin.

Embodiment 12. The method of any one of Embodiments 1-11, wherein said method comprises administering said composition comprising said senolytic agent to said donor mammal providing said graft before said graft is obtained from said donor mammal.

Embodiment 13. The method of Embodiment 12, wherein said graft has improved function or survival within said recipient mammal as compared to a comparable graft obtained from a comparable control donor not administered said composition.

Embodiment 14. The method of any one of Embodiments 1-11, wherein said method comprises administering said composition to said recipient mammal.

Embodiment 15. The method of Embodiment 14, wherein said composition is administered to said recipient mammal before said graft is provided to said recipient.

Embodiment 16. The method of any one of Embodiments 14-15, wherein said composition is administered to said recipient mammal after said graft is provided to said recipient.

Embodiment 17. The method of any one of Embodiments 14-16, wherein said composition is administered to said recipient mammal at the same time that said graft is provided to said recipient.

Embodiment 18. The method of any one of Embodiments 14-17, wherein said graft has improved function or survival within said recipient mammal as compared to a comparable graft provided to a comparable recipient mammal not administered said composition.

Embodiment 19. The method of any one of Embodiments 1-11, wherein said method comprises contacting said graft with said composition.

Embodiment 20. The method of Embodiment 19, wherein said graft has improved function or survival within said recipient mammal as compared to a comparable graft not contacted with said composition.

Embodiment 21. The method of any one of Embodiments 1-20, wherein said method comprises administering said composition comprising said senolytic agent to said donor mammal providing said graft before said graft is obtained from said donor mammal, administering said composition to said recipient mammal, and contacting said graft with said composition.

Embodiment 22. The method of any one of Embodiments 1-21, wherein said method further comprises (c1) administering a composition comprising a toll-like receptor 9 (TLR9) antagonist to a donor mammal providing said graft before said graft is obtained from said donor mammal, (c2) administering said composition comprising said TLR9 antagonist to said recipient mammal, or (c3) contacting said graft with said composition comprising said TLR9 antagonist.

Embodiment 23. The method of Embodiment 22, wherein said TLR9 antagonist is ODN 2088.

Embodiment 24. A non-human mammalian model for aging or transplantation, wherein said model is a non-human mammal transplanted with a population of senescent cells.

Embodiment 25. The model of Embodiment 24, wherein said population comprises less than 5 million cells.

Embodiment 26. The model of any one of Embodiments 24-25, wherein said population of senescent cells was transplanted into a young wild type non-human mammal, an old wild type non-human mammal, a young wild type high fat fed non-human mammal, or an immunodeficient non-human mammal.

Embodiment 27. The model of any one of Embodiments 24-26, wherein said non-human mammal is a mouse.

Embodiment 28. The model of any one of Embodiments 24-27, wherein said model has a reduced survival time as compared to a comparable non-human mammal not transplanted with said population of senescent cells.

Embodiment 29. The model of any one of Embodiments 24-28, wherein said model comprises an exogenous graft.

Embodiment 30. The method of Embodiment 29, wherein said exogenous graft exhibits an inferior performance within said model as compared to a comparable graft within a non-human mammal not transplanted with said population of senescent cells.

Embodiment 31. A method for identifying an agent having the ability to reduce the effect of aging within a mammal, wherein said method comprises: (a) administering a test agent to a model of any one of claims 24-30, and (b) determining whether or not said test agent reduces an effect of aging within said model.

Embodiment 32. The method of Embodiment 31, wherein said test agent reduce an effect of aging within said model, thereby identifying said test agent as being said agent.

Embodiment 33. A method for identifying an agent having the ability to improve the performance of a transplanted graft within a mammal, wherein said method comprises: (a) administering a test agent to a model of any one of Embodiments 29-30, and (b) determining whether or not said test agent improves the performance of said exogenous graft within said model.

Embodiment 34. The method of Embodiment 33, wherein said test agent improves the performance of said exogenous graft within said model, thereby identifying said test agent as being said agent.

Embodiment 35. A method for detecting senescent cells, wherein said method comprises determining the presence or absence of cell-free mitochondrial DNA (cf-mt-DNA) in a sample, wherein the presence of cf-mt-DNA in said sample indicates that said sample contains senescent cells, and wherein the absence of cf-mt-DNA in said sample indicates that said sample lacks senescent cells.

Embodiment 36. The method of Embodiment 35, wherein said determining step comprises a PCR technique.

Embodiment 37. The method of Embodiment 36, wherein said PCR technique is real time PCR.

Embodiment 38. The method of any one of Embodiments 35-37, wherein said sample is obtained from a recipient mammal with a graft.

Embodiment 39. The method of any one of Embodiments 35-37, wherein said sample is obtained from a donor mammal providing a graft to be transplanted into a recipient mammal.

Embodiment 40. The method of any one of Embodiments 35-37, wherein said sample is obtained from a graft to be transplanted into a recipient mammal.

Embodiment 41. The method of any one of Embodiments 38-40, wherein said mammal is a human.

Embodiment 42. The method of any one of Embodiments 38-41, wherein said graft is a tissue graft.

Embodiment 43. The method of Embodiment 42, wherein said tissue graft is bone marrow.

Embodiment 44. The method of any one of Embodiments 38-41, wherein said graft is an organ graft.

Embodiment 45. The method of any one of Embodiments 38-41, wherein said graft is a population of cells not in the form of tissue or an organ.

Embodiment 46. The method of Embodiment 45, wherein said population of cells comprises hematopoietic stem cells or blood.

Embodiment 47. The method of any one of Embodiments 35-46, wherein said method comprises determining the presence of said cf-mt-DNA in said sample.

Embodiment 48. The method of any one of Embodiments 35-46, wherein said method comprises determining the absence of said cf-mt-DNA in said sample.

Embodiment 49. A method for detecting senescent cells, wherein said method comprises determining the presence or absence of an elevated level of cf-mt-DNA in a sample, wherein the presence of said elevated level of cf-mt-DNA in said sample indicates that said sample contains senescent cells, and wherein the absence of said elevated level of cf-mt-DNA in said sample indicates that said sample lacks senescent cells.

Embodiment 50. The method of Embodiment 49, wherein said determining step comprises a PCR technique.

Embodiment 51. The method of Embodiment 50, wherein said PCR technique is real time PCR.

Embodiment 52. The method of any one of Embodiments 49-51, wherein said sample is obtained from a recipient mammal with a graft.

Embodiment 53. The method of any one of Embodiments 49-51, wherein said sample is obtained from a donor mammal providing a graft to be transplanted into a recipient mammal.

Embodiment 54. The method of any one of Embodiments 49-51, wherein said sample is obtained from a graft to be transplanted into a recipient mammal.

Embodiment 55. The method of any one of Embodiments 49-54, wherein said mammal is a human.

Embodiment 56. The method of any one of Embodiments 49-55, wherein said graft is a tissue graft.

Embodiment 57. The method of Embodiment 56, wherein said tissue graft is bone marrow.

Embodiment 58. The method of any one of Embodiments 49-55, wherein said graft is an organ graft.

Embodiment 59. The method of any one of c1 Embodiments aims 49-55, wherein said graft is a population of cells not in the form of tissue or an organ.

Embodiment 60. The method of Embodiment 59, wherein said population of cells comprises hematopoietic stem cells or blood.

Embodiment 61. The method of any one of Embodiments 49-60, wherein said method comprises determining the presence of said elevated level of cf-mt-DNA in said sample.

Embodiment 62. The method of any one of Embodiments 49-60, wherein said method comprises determining the absence of said elevated level of cf-mt-DNA in said sample.

Embodiment 63. The method of any one of Embodiments 49-62, wherein said sample is liquid.

Embodiment 64. The method of Embodiment 63, wherein said elevated level is greater than 20,000 copies per mL of sample.

Embodiment 65. A method for providing a recipient mammal with a graft, wherein said method comprises: (a1) administering a composition comprising a toll-like receptor 9 (TLR9) antagonist to a donor mammal providing said graft before said graft is obtained from said donor mammal, (a2) administering said composition to said recipient mammal, or (a3) contacting said graft with said composition, and (b) providing said recipient mammal with said graft.

Embodiment 66. The method of Embodiment 65, wherein said recipient mammal is a human.

Embodiment 67. The method of any one of Embodiments 65-66, wherein said graft is a graft from a human donor.

Embodiment 68. The method of Embodiment 67, wherein said human donor is over 55 years of age.

Embodiment 69. The method of any one of Embodiments 65-68, wherein said graft is a tissue graft.

Embodiment 70. The method of Embodiment 69, wherein said tissue graft is bone marrow.

Embodiment 71. The method of any one of Embodiments 65-68, wherein said graft is an organ graft.

Embodiment 72. The method of any one of Embodiments 65-68, wherein said graft is a population of cells not in the form of tissue or an organ.

Embodiment 73. The method of Embodiment 72, wherein said population of cells comprises hematopoietic stem cells or blood.

Embodiment 74. The method of any one of Embodiments 65-73, wherein said TLR9 antagonist is ODN 2088, SD-101, IMO-2125, CPG10101, or chloroquine.

Embodiment 75. The method of any one of Embodiments 65-74, wherein said composition comprises ODN 2088.

Embodiment 76. The method of any one of Embodiments 65-75, wherein said method comprises administering said composition to said donor mammal providing said graft before said graft is obtained from said donor mammal.

Embodiment 77. The method of Embodiment 76, wherein said graft has improved function or survival within said recipient mammal as compared to a comparable graft obtained from a comparable control donor not administered said composition.

Embodiment 78. The method of any one of Embodiments 65-75, wherein said method comprises administering said composition to said recipient mammal.

Embodiment 79. The method of Embodiment 78, wherein said composition is administered to said recipient mammal before said graft is provided to said recipient.

Embodiment 80. The method of any one of Embodiments 78-79, wherein said composition is administered to said recipient mammal after said graft is provided to said recipient.

Embodiment 81. The method of any one of Embodiments 78-80, wherein said composition is administered to said recipient mammal at the same time that said graft is provided to said recipient.

Embodiment 82. The method of any one of Embodiments 78-81, wherein said graft has improved function or survival within said recipient mammal as compared to a comparable graft provided to a comparable recipient mammal not administered said composition.

Embodiment 83. The method of any one of Embodiments 65-75, wherein said method comprises contacting said graft with said composition.

Embodiment 84. The method of Embodiment 83, wherein said graft has improved function or survival within said recipient mammal as compared to a comparable graft not contacted with said composition.

Embodiment 85. The method of any one of Embodiments 65-84, wherein said method comprises administering said composition to said donor mammal providing said graft before said graft is obtained from said donor mammal, administering said composition to said recipient mammal, and contacting said graft with said composition.

Embodiment 86. The method of any one of Embodiments 65-85, wherein said method further comprises (c1) administering a composition comprising a senotherapeutic agent to a donor mammal providing said graft before said graft is obtained from said donor mammal, (c2) administering said composition comprising said senolytic agent to said recipient mammal, or (c3) contacting said graft with said composition comprising senolytic agent.

Embodiment 87. The method of Embodiment 86, wherein said senotherapeutic agent is a senolytic agent.

Embodiment 88. The method of Embodiment a87, wherein said senolytic agent is dasatinib or quercetin.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for providing a recipient mammal with a graft, wherein said method comprises:
(a1) administering a composition comprising a senolytic agent to a donor mammal providing said graft before said graft is obtained from said donor mammal, (a2) administering said composition to said recipient mammal, or (a3) contacting said graft with said composition, and
(b) providing said recipient mammal with said graft.

2. The method of claim 1, wherein said recipient mammal is a human.

3. The method of claim 1, wherein said graft is a graft from a human donor.

4. (canceled)

5. The method of claim 1, wherein said graft is a tissue graft, an organ graft, or a population of cells not in the form of tissue or an organ.

6. The method of claim 5, wherein said tissue graft is bone marrow.

7. The method of claim 5, wherein said population of cells comprises hematopoietic stem cells or blood.

8. The method of claim 1, wherein said senolytic agent is dasatinib or quercetin.

9-12. (canceled)

13. A non-human mammalian model for aging or transplantation, wherein said model is a non-human mammal transplanted with a population of senescent cells.

14. (canceled)

15. The model of claim 13, wherein said population of senescent cells was transplanted into a young wild type non-human mammal, an old wild type non-human mammal, a young wild type high fat fed non-human mammal, or an immunodeficient non-human mammal.

16. The model of claim 13, wherein said non-human mammal is a mouse.

17. The model of claim 13, wherein said model comprises an exogenous graft.

18-36. (canceled)

37. A method for providing a recipient mammal with a graft, wherein said method comprises:
(a1) administering a composition comprising a toll-like receptor 9 (TLR9) antagonist to a donor mammal providing said graft before said graft is obtained from said donor mammal, (a2) administering said composition to said recipient mammal, or (a3) contacting said graft with said composition, and
(b) providing said recipient mammal with said graft.

38. The method of claim 37, wherein said recipient mammal is a human.

39. The method of claim 37, wherein said graft is a graft from a human donor.

40. (canceled)

41. The method of claim 37, wherein said graft is a tissue graft, an organ graft, or a population of cells not in the form of tissue or an organ.

42. The method of claim 41, wherein said tissue graft is bone marrow.

43. The method of claim 41, wherein said population of cells comprises hematopoietic stem cells or blood.

44. The method of claim 37, wherein said TLR9 antagonist is ODN 2088, SD-101, IMO-2125, CPG10101, or chloroquine.

45. The method of claim 37, wherein said method comprises administering said composition to said donor mammal providing said graft before said graft is obtained from said donor mammal, administering said composition to said recipient mammal, or contacting said graft with said composition.

46. The method of claim 37, wherein said method comprises administering said composition to said donor mammal providing said graft before said graft is obtained from said donor mammal, administering said composition to said recipient mammal, and contacting said graft with said composition.

47-49. (canceled)
